(19) United States
(12) Patent Application Publication
(10) Pub. No.: US 2005/0250137 A1
(43) Pub. Date: Nov. 10, 2005

(54) Molecular targets of cancer and aging

(76) Inventors: Michael A. Tainsky, West Bloomfield, MI (US); Sorin Draghici, Troy, MI (US); Olga I. Studitskaia, Edison, NJ (US)

Correspondence Address: Amy E. Rinaldo;KOHN & ASSOCIATES, PLLC, Suite 410, 30500 Northwestern Highway, Farmington Hills, MI, 48334, US

(21) Appl. No.: 11/085,440

(22) Filed: Mar. 21, 2005

Related U.S. Application Data
(63) Continuation-in-part of application No. PCT/US03/29624, filed on Sep. 22, 2003.
(60) Provisional application No. 60/412,228, filed on Sep. 20, 2002.
(60) Provisional application No. 60/478,548, filed on Jun. 13, 2003.

(51) Int. Cl. C12Q 1/68; G06F 19/00; G01N 33/48; G01N 33/50

(52) U.S. Cl. 435/6; 702/20

(57) ABSTRACT

A diagnostic tool for use in diagnosing diseases, the tool is a detector for detecting a presence of an array of markers being used to determine gene expression changes that are related to cellular immortalization, the presence of the markers being indicative of a specific disease and the markers and treatments found by the tool. A tool for interpreting results of a microarray, wherein the tool is a computer program for analyzing the results of microrarrays. A method of creating an array of markers for diagnosing the presence of disease by microarraying sera obtained from a patient to obtain molecular markers of disease and detecting markers that are present only in the sera of patients with a specific disease thereby detecting molecular markers being used to determine gene expression changes that are related to cellular immortalization and for use in diagnosing disease.

### CROSS-REFERENCE TO RELATED APPLICATIONS

This application is a Continuation-in-Part of PCT/US03/29624, which claims the benefit of priority under 35 U.S.C. Section 119(e) of U.S. Provisional Patent Application Nos. 60/412,228, filed Sep. 20, 2002 and 60/478,548, Filed Jun. 13, 2003, which is incorporated herein by reference.

### BACKGROUND OF THE INVENTION

1. Technical Field

The present invention relates to molecular targets of cancer and aging. More specifically, the present invention relates to a microarray for use in determining molecular targets of cancer and aging.

2. Description of the Related Art

It is commonly known in the art that genetic mutations can be used for detecting cancer. For example, the tumorigenic process leading to colorectal carcinoma formation involves multiple genetic alterations (Fearon et al (1990) Cell 61, 759-767). Tumor suppressor genes such as p53, DCC and APC are frequently inactivated in colorectal carcinomas, typically by a combination of genetic deletion of one allele and point mutation of the second allele (Baker et al (1989) Science 244, 217-221; Fearon et al (1990) Science 247, 49-56; Nishisho et al (1991) Science 253, 665-669; and Groden et al (1991) Cell 66, 589-600). Recently, mutation of two mismatch repair genes that regulate genetic stability was associated with a form of familial colon cancer (Fishel et al (1993) Cell 75, 1027-1038; Leach et al (1993) Cell 75, 1215-1225; Papadopoulos et al (1994) Science 263, 1625-1629; and Bronner et al (1994) Nature 368, 258-261). Proto-oncogenes such as myc and ras are altered in colorectal carcinomas, with c-myc RNA being overexpressed in as many as 65% of carcinomas (Erisman et al (1985) Mol. Cell. Biol. 5, 1969-1976), and ras activation by point mutation occurring in as many as 50% of carcinomas (Bos et al (1987) Nature 327, 293-297; and Forrester et al (1987) Nature 327, 298-303). Other proto-oncogenes, such as myb and neu are activated with a much lower frequency (Alitalo et al (1984) Proc. Natl. Acad. Sci. USA 81, 4534-4538; and D'Emilia et al (1989) Oncogene 4, 1233-1239). No common series of genetic alterations is found in all colorectal tumors, suggesting that a variety of such combinations can be able to generate these tumors.

Increased tyrosine phosphorylation is a common element in signaling pathways that control cell proliferation. The deregulation of protein tyrosine kinases (PTKS) through overexpression or mutation has been recognized as an important step in cell transformation and tumorigenesis, and many oncogenes encode PTKs (Hunter (1989) in oncogenes and the Molecular Origins of Cancer, ed. Weinberg (Cold Spring Harbor Laboratory Press, Cold Spring Harbor, N.Y.), pp. 147-173). Numerous studies have addressed the involvement of PTKs in human tumorigenesis. Activated PTKs associated with colorectal carcinoma include c-neu (amplification), trk (rearrangement), and c-src and c-yes (mechanism unknown) (D'Emilia et al (1989), ibid; Martin-Zanca et al (1986) Nature 3, 743-748; Bolen et al (1987) Proc. Natl. Acad. Sci. USA 84, 2251-2255; Cartwright et al (1989) J. Clin. Invest. 83, 2025-2033; Cartwright et al (1990) Proc. Natl. Acad. Sci. USA 87, 558-562; Talamonti et al (1993) J. Clin. Invest. 91, 53-60; and Park et al (1993) Oncogene 8, 2627-2635).

Obviously, protein tyrosine phosphatases (PTPs) are also intimately involved in regulating cellular phosphotyrosine levels. The growing family of PTPs consists of non-receptor and receptor-like enzymes (for review see Charbonneau et al (1992) Annu. Rev. Cell. Biol. 8, 463493; and Pot et al (1992) Biochim. Biophys. Acta 1136, 35-43). All share a conserved catalytic domain, which in the non-receptor PTPs is often associated with proximal or distal sequences containing regulatory elements directing protein-protein interaction, intracellular localization, or PTP stability. The receptor like PTPs usually contain two catalytic domains in their intracellular region, and in addition have a transmembrane region and heterogeneous extracellular regions. The extreme diversity of the extracellular region, compared to the relatively conserved intracellular portion of these enzymes, suggests that these PTPs are regulated by specific extracellular factors, few of which have been identified. Some PTPs can act in opposition to PTKs. For example, the nonreceptor PTP 1B and TC-PTP can reverse or block cell transformation induced by the oncogenic tyrosine kinases neu or v-fms, while another non-receptor PTP (known as 3HC134, CL100, HVH1, PAC-1, erp, or MKP-1) can reverse the PTK-mediated activation of a central signaling enzyme, MAP kinase (Brown-Shimer et al (1992) Cancer Res. 52, 478-482; Zander et al (1993) Oncogene 8, 1175-1182; Sun et al (1993) Cell 75, 487-493; and Ward et al (1994) Nature 367, 651-654). Conversely, other PTPs can act in conjunction with PTKs. Two receptor-like PTPs, PTPa and CD45, respectively activate the tyrosine kinases c-src or Ick and fyn while the non-receptor SH-PTP2 (PTP 1D, PTP-2C, Syp) positively transduces a mitogenic signal from the PDGF receptor tyrosine kinase to ras (WP 94/01119; Zheng et al (1992) Nature 359, 336-339; den Hertog et al (1993) EMUB J. 12, 3789-3798; Mustelin et al (1989) Proc. Natl. Acad. Sci. USA 86, 6302-6306; Ostergaard et al (1989) Proc. Natl. Acad. Sci. USA 86, 8959-8963; Cahir McFarland et al (1989) Proc. Natl. Acad. Sci. USA 90, 1402-1406; and Li et al (1994) Mol. Cell. Biol. 14, 509-517).

Very few studies have examined alterations in PTP expression or activity that can be associated with tumorigenesis. As indicated above, two PTP-related mechanisms, either the inactivation or the overactivation of a PTP, could increase cellular phosphotyrosine levels and result in uncontrolled cell proliferation and tumorigenesis. In relation to PTP inactivation, it is of interest that the gene encoding receptor-like PTP7 is situated on a region of chromosome 3 that is often lost in renal and lung carcinomas, and that a PTPW allele is lost in some renal carcinoma and lung carcinoma cell lines (LaForgia et al (1991) Proc. Natl. Acad. Sci. USA 88, 5036-5040). As regards PTP overactivation, it has been shown that when PTPa is overexpressed in rat embryo fibroblasts, cell transformation occurs and the cells are tumorigenic in nude mice (WO 94/01119 and Zheng et al (1992), ibid). PTPα is a receptor-like enzyme with a short, unique extracellular domain and two tandem catalytic domains (WO 92/01050; Matthews et al (1990) Proc. Natl. Acad. Sci. USA 87, 4444-4448; Sap et al (1990) Proc. Natl. Acad. Sci. USA 87, 6112-6116; and Krueger et al (1990) EMBO J. 9, 3241-3252). Compared to many other receptor-like PTPs with a restricted and lineage-specific expression, PTPα is widely expressed (Sap et al (1990), ibid and Krueger et al (1990), ibid).

Mutations, such as those disclosed above can be useful in detecting cancer. However, there have been few advancements that can repeatably be used in diagnosing cancer prior to the existence of a tumor. For example, breast cancer, which is by far the most common form of cancer in women, is the second leading cause of cancer death in humans. Despite many recent advances in diagnosing and treating breast cancer, the prevalence of this disease has been steadily rising at a rate of about 1% per year since 1940. Today, the likelihood that a women living in North America can develop breast cancer during her lifetime is one in eight.

The current widespread use of mammography has resulted in improved detection of breast cancer. Nonetheless, the death rate due to breast cancer has remained unchanged at about 27 deaths per 100,000 women. All too often, breast cancer is discovered at a stage that is too far advanced, when therapeutic options and survival rates are severely limited. Accordingly, more sensitive and reliable methods are needed to detect small (less than 2 cm diameter), early stage, in situ carcinomas of the breast. Such methods should significantly improve breast cancer survival, as suggested by the successful employment of Papinicolou smears for early detection and treatment of cervical cancer.

In addition to the problem of early detection, there remain serious problems in distinguishing between malignant and benign breast disease, in staging known breast cancers, and in differentiating between different types of breast cancers (e.g. estrogen dependent versus non-estrogen dependent tumors). Recent efforts to develop improved methods for breast cancer detection, staging and classification have focused on a promising array of so-called cancer “markers.” Cancer markers are typically proteins that are uniquely expressed (e.g. as a cell surface or secreted protein) by cancerous cells, or are expressed at measurably increased or decreased levels by cancerous cells compared to normal cells. Other cancer markers can include specific DNA or RNA sequences marking deleterious genetic changes or alterations in the patterns or levels of gene expression associated with particular forms of cancer.

A large number and variety of breast cancer markers have been identified to date, and many of these have been shown to have important value for determining prognostic and/or treatment-related variables. Prognostic variables are those variables that serve to predict disease outcome, such as the likelihood or timing of relapse or survival. Treatment-related variables predict the likelihood of success or failure of a given therapeutic plan. Certain breast cancer markers clearly serve both functions. For example, estrogen receptor levels are predictive of relapse and survival for breast cancer patients, independent of treatment, and are also predictive of responsiveness to endocrine therapy. Pertschuk et al., Cancer 66: 1663-1670, 1990; Parl and Posey, Hum. Pathol. 19: 960-966, 1988; Kinsel et al., Cancer Res. 49: 1052-1056, 1989; Anderson and Poulson Cancer 65: 1901-1908, 1989.

The utility of specific breast cancer markers for screening and diagnosis, staging and classification, monitoring and/or therapy purposes depends on the nature and activity of the marker in question. For general reviews of breast cancer markers, see Porter-Jordan et al., Hematol. Oncol. Clin. North Amer. 8: 73-100, 1994; and Greiner, Pharmaceutical Tech., May, 1993, pp. 2844. As reflected in these reviews, a primary focus for developing breast cancer markers has centered on the overlapping areas of tumorigenesis, tumor growth and cancer invasion. Tumorigenesis and tumor growth can be assessed using a variety of cell proliferation markers (for example Ki67, cyclin D1, and proliferating cell nuclear antigen (PCNA)), some of which can be important oncogenes as well. Tumor growth can also be evaluated using a variety of growth factor and hormone markers (for example estrogen, epidermal growth factor (EGF), erbB-2, transforming growth factor (TGF)a), which can be overexpressed, underexpressed or exhibit altered activity in cancer cells. By the same token, receptors of autocrine or exocrine growth factors and hormones (for example insulin growth factor (IGF) receptors, and EGF receptor) can also exhibit changes in expression or activity associated with tumor growth. Lastly, tumor growth is supported by angiogenesis involving the elaboration and growth of new blood vessels and the concomitant expression of angiogenic factors that can serve as markers for tumorigenesis and tumor growth.

In addition to tumorigenic, proliferation, and growth markers, a number of markers have been identified that can serve as indicators of invasiveness and/or metastatic potential in a population of cancer cells. These markers generally reflect altered interactions between cancer cells and their surrounding microenvironment. For example, when cancer cells invade or metastasize, detectable changes can occur in the expression or activity of cell adhesion or motility factors, examples of which include the cancer markers Cathepsin D, plasminogen activators, collagenases and other factors. In addition, decreased expression or overexpression of several putative tumor “suppressor” genes (for example nm23, p53 and rb) has been directly associated with increased metastatic potential or deregulation of growth predictive of poor disease outcome.

In summary, the evaluation of proliferation markers, oncogenes, growth factors and growth factor receptors, angiogenic factors, proteases, adhesion factors and tumor suppressor genes, among other cancer markers, can provide important information concerning the risk, presence, status or future behavior of cancer in a patient. Determining the presence or level of expression or activity of one or more of these cancer markers can aid in the differential diagnosis of patients with uncertain clinical abnormalities, for example by distinguishing malignant from benign abnormalities. Furthermore, in patients presenting with established malignancy, cancer markers can be useful to predict the risk of future relapse, or the likelihood of response in a particular patient to a selected therapeutic course. Even more specific information can be obtained by analyzing highly specific cancer markers, or combinations of markers, which can predict responsiveness of a patient to specific drugs or treatment options.

Methods for detecting and measuring cancer markers have been recently revolutionized by the development of immunological assays, particularly by assays that utilize monoclonal antibody technology. Previously, many cancer markers could only be detected or measured using conventional biochemical assay methods, which generally require large test samples and are therefore unsuitable in most clinical applications. In contrast, modern immunoassay techniques can detect and measure cancer markers in relatively much smaller samples, particularly when monoclonal antibodies that specifically recognize a targeted marker protein are used. Accordingly, it is now routine to assay for the presence or absence, level, or activity of selected cancer markers by immunohistochemically staining tissue specimens obtained via conventional biopsy methods. Because of the highly sensitive nature of immunohistochemical staining, these methods have also been successfully employed to detect and measure cancer markers in smaller, needle biopsy specimens which require less invasive sample gathering procedures compared to conventional biopsy specimens. In addition, other immunological methods have been developed and are now well known in the art that allow for detection and measurement of cancer markers in non-cellular samples such as serum and other biological fluids from patients. The use of these alternative sample sources substantially reduces the morbidity and costs of assays compared to procedures employing conventional biopsy samples, which allows for application of cancer marker assays in early screening and low risk monitoring programs where invasive biopsy procedures are not indicated.

For the purpose of cancer evaluation, the use of conventional or needle biopsy samples for cancer marker assays is often undesirable, because a primary goal of such assays is to detect the cancer before it progresses to a palpable or detectable tumor stage. Prior to this stage, biopsies are generally contraindicated, making early screening and low risk monitoring procedures employing such samples untenable. Therefore, there is general need in the art to obtain samples for cancer marker assays by less invasive means than biopsy, for example by serum withdrawal.

Efforts to utilize serum samples for cancer marker assays have met with limited success, largely because the targeted markers are either not detectable in serum, or because telltale changes in the levels or activity of the markers cannot be monitored in serum. In addition, the presence of cancer markers in serum probably occurs at the time of micro-metastasis, making serum assays less useful for detecting pre-metastatic disease.

Previous attempts to develop non-invasive breast cancer marker assays utilizing mammary fluid samples have included studies of mammary fluid obtained from patients presenting with spontaneous nipple discharge. In one of these studies, conducted by Inaji et al., Cancer 60: 3008-3013, 1987, levels of the breast cancer marker carcinoembryonic antigen (CEA) were measured using conventional, enzyme linked immunoassay (ELISA) and sandwich-type, monoclonal immunoassay methods. These methods successfully and reproducibly demonstrated that CEA levels in spontaneously discharged mammary fluid provide a sensitive indicator of nonpalpable breast cancer. In a subsequent study, also by Inaji et al., Jpn. J. Clin. Oncol. 19: 373-379, 1989, these results were expanded using a more sensitive, dry chemistry, dot-immunobinding assay for CEA determination. This latter study reported that elevated CEA levels occurred in 43% of patients tested with palpable breast tumors, and in 73% of patients tested with nonpalpable breast tumors. CEA levels in the discharged mammary fluid were highly correlated with intratumoral CEA levels, indicating that the level of CEA expression by breast cancer cells is closely reflected in the mammary fluid CEA content. Based on these results, the authors concluded that immunoassays for CEA in spontaneously discharged mammary fluid are useful for screening nonpalpable breast cancer.

Although the evaluation of mammary fluid has been shown to be a useful method for screening nonpalpable breast cancer in women who experience spontaneous nipple discharge, the rarity of this condition renders the methods of Inaji et al, inapplicable to the majority of women who are candidates for early breast cancer screening. In addition, the first Inaji report cited above determined that certain patients suffering spontaneous nipple discharge secrete less than 10.mu.l of mammary fluid, which is a critically low level for the ELISA and sandwich immunoassays employed in that study. It is likely that other antibodies used to assay other cancer markers can exhibit even lower sensitivity than the anti-CEA antibodies used by Inaji and coworkers, and can therefore not be adaptable or sensitive enough to be employed even in dry chemical immunoassays of small samples of spontaneously discharged mammary fluid.

In view of the above, an important need exists in the art for more widely applicable, non-invasive methods and materials to obtain biological samples for use in evaluating, diagnosing and managing breast and other diseases including cancer, particularly for screening early stage, nonpalpable tumors. A related need exists for methods and materials that utilize such readily obtained biological samples to evaluate, diagnose, and manage disease, particularly by detecting or measuring selected molecular cancer markers to provide highly specific, cancer prognostic and/or treatment-related information, and to diagnose and manage pre-cancerous conditions, cancer susceptibility, bacterial, and other infections, and other diseases.

### SUMMARY OF THE INVENTION

According to the present invention, there is provided a diagnostic tool for use in diagnosing diseases, the tool is a detector for detecting a presence of an array of markers indicative of a specific disease and the marker and treatments found therefrom. A tool for interpreting results of a microarray, wherein the tool is a computer program for analyzing the results of microrarrays. A method of creating an array of markers for diagnosing the presence of disease by microarraying sera obtained from a patient to obtain molecular markers of disease and detecting markers that are present only in the sera of patients with a specific disease thereby detecting molecular markers for use in diagnosing disease.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1 is a photograph showing 5-aza-CdR mediated up-regulation of STAT1α;

FIGS. 2A and B are photographs showing the hierarchical clustering of gene expression using GeneSight software; and

FIG. 3 is a photograph showing 5-aza-CdR mediated up-regulation of p16^{INK4a} protein.

FIG. 4 is a photograph showing the Western blot analysis of MDAH041 and MDAH087 cell lines, wherein UT: untreated; 5A: 5-aza-dC; 041-PC: precrisis MDAH041; 041-IM: immortal MDAH041; 087-PC: precrisis MDAH087; 087-N: MDAH087-N; 087-1: MDAH087-1; 087-10: MDAH087-10, and tubulin is a loading control;

FIG. 5a is a photograph showing hierarchical clustering of gene expression data in MDAH041, MDAH087-N, MDAH087-1, and MDAH087-10, wherein each row represents a probe on the HGU95Av2 GeneChip®, each column represents the average comparisons of each cell line. 041-IM: immortal MDAH041; 087-N: MDAH087-N; 087-1: MDAH087-1; 087-10: MDAH087-10;

FIG. 5b is a graph showing multidimensional scaling analysis of gene expression data in MDAH041, MDAH087-N, MDAH087-1, and MDAH087-10, wherein 5A: upregulated in 5-aza-dC-treated immortal cells versus untreated immortal cells; UT: Untreated, downregulated in immortal cells versus precrisis cells. 041-IM: immortal MDAH041; 087-N: MDAH087-N; 087-1: MDAH087-1; 087-10: MDAH087-10;

FIG. 6A through C are graphs depicting GoMiner analysis of differentially regulated genes in all four immortal LFS cell lines, wherein the genes, which were dysregulated (up- or downregulated) during immortalization and 5-aza-dC treatment in MDAH041, MDAH087-N, MDAH087-1, MDAH087-10 cells were analyzed by GoMiner according to biological process (FIG. 6A), cellular component (FIG. 6B) and molecular function (FIG. 6C). The first layer GO categories were plotted based on their −log₁₀(p-value). IM: genes dysregulated during immortalization; 5A: genes dysregulated during 5-aza-dC treatment of immortal cells. p-values, which were smaller than 0.0001, were replaced with 0.0001 to get a viewable range of the plot. GO categories identified to be significant by corrected p-value were marked by *;

FIG. 7 is a series of chromosome ideograms of genes differentially expressed genes in all four immortal LFS cell lines, fragile sites and imprinted genes, wherein the ideograms from left to right, for each chromosome, are reference ideogram of cytogenetic regions (R), ideogram of genes decreased during immortalization (D), ideogram of imprinted genes (I), and ideogram of genes increased after 5-aza-dC treatment (5A). The colored lines represent location of genes. Fragile sites are represented by a dot (F). Genes that are epigenetically regulated during immortalization are labeled on the ideograms;

FIG. 8 is a series of chromosome ideograms depicting the localization of genes, in the four immortal LFS cell lines, with increased expression during immortalization

FIG. 9 is a series of chromosome ideograms depicting the localization of genes, in the four immortal LFS cell lines, with decreased expression during immortalization

FIG. 10 is a series of chromosome ideograms depicting the localization of genes, in the four immortal LFS cell lines, with increased expression after 5-aza-dC treatment

FIG. 11 is a series of chromosome ideograms depicting the localization of genes, in the four immortal LFS cell lines, with decreased expression after 5-aza-dC treatment

FIG. 12 is a series of chromosome ideograms depicting the localization of genes, in the four immortal LFS cell lines, with increased expression during immortalization and decreased expression after 5-aza-dC treatment

FIG. 13 is a series of chromosome ideograms depicting the localization of genes, in the four immortal LFS cell lines, with decreased expression during immortalization and increased expression after 5-aza-dC treatment

### DESCRIPTION OF THE INVENTION

Generally, the present invention relates to a method of determining molecular targets of cancer and aging and the targets obtained by the same. The method includes analyzing the results obtained from a microarray that is used for determining the molecular targets of cancer and aging.

The microarray of the present invention is any microarray that can be used to determine gene expression changes that are related to cellular immortalization. The gene expression changes that are determined as a result of the microarray are then compared to the gene expression changes due to variations in gene expression after inhibiting a fundamental pathway in the immortalization process. The genes expression changes relate to early events in the cellular progression to cancer both for molecular targets and diagnostic targets.

More specifically, the pathway is affected by inhibiting a fundamental aspect of the pathway; for example, inhibition of DNA methylation in immortal fibroblast cells. The pathway can be a growth suppressor, a growth promotor, or is otherwise involved in cell growth or proliferation. The results of the comparison of the gene expression changes are compared to identify genes that are regulated in both conditions, thereby identifying genes that are molecular targets of cancer and aging.

The use of microarray technology allows for the study of a complex interplay of genes and other genetic material, simultaneously. The pattern of genes expressed in a cell is characteristic of its state. Virtually all differences in cell state correlate with changes in mRNA levels of genes. Generally, microarray technology involves obtaining complementary genetic material to genetic material of interest and laying out the complementary genetic material in microscopic quantities on solid surfaces at defined positions. Genetic material from samples is then eluted over the surface and complementary genetic material binds thereto. The presence of bound genetic material then is detected by fluorescence following laser excitation.

By “support or surface” as used herein, the term is intended to include, but is not limited to a solid phase, which is a porous or non-porous water insoluble material that can have any one of a number of shapes, such as strip, rod, particle, including beads and the like. Suitable materials are well known in the art and are described in, for example, Ullman, et al. U.S. Pat. No. 5,185,243, columns 10-11, Kum, et al., U.S. Pat. No. 4,868,104, column 6, lines 21-42 and Milburn, et al., U.S. Pat. No. 4,959,303, column 6, lines 14-31 that are incorporated herein by reference. Binding of ligands and receptors to the support or surface can be accomplished by well-known techniques, readily available in the literature. See, for example, “Immobilized Enzymes,” Ichiro Chibata, Halsted Press, New York (1978) and Cuatrecasas, J. Biol. Chem. 245:3059 (1970). Whatever type of solid support is used, it must be treated so as to have bound to its surface either a receptor or ligand that directly or indirectly binds the antigen. Typical receptors include antibodies, intrinsic factor, specifically reactive chemical agents such as sulfhydryl groups that can react with a group on the antigen, and the like. For example, avidin or streptavidin can be covalently bound to spherical glass beads of 0.5-1.5 mm and used to capture a biotinylated antigen.

The “molecular markers” that are isolated can be any marker known to those of skill in the art to be related to cancer or aging. The markers can be any detectable marker that is altered due to the present of cancer or the onset of aging. Examples of such markers include, but are not limited to, IFN pathway genes and molecular targets involved in immortalization.

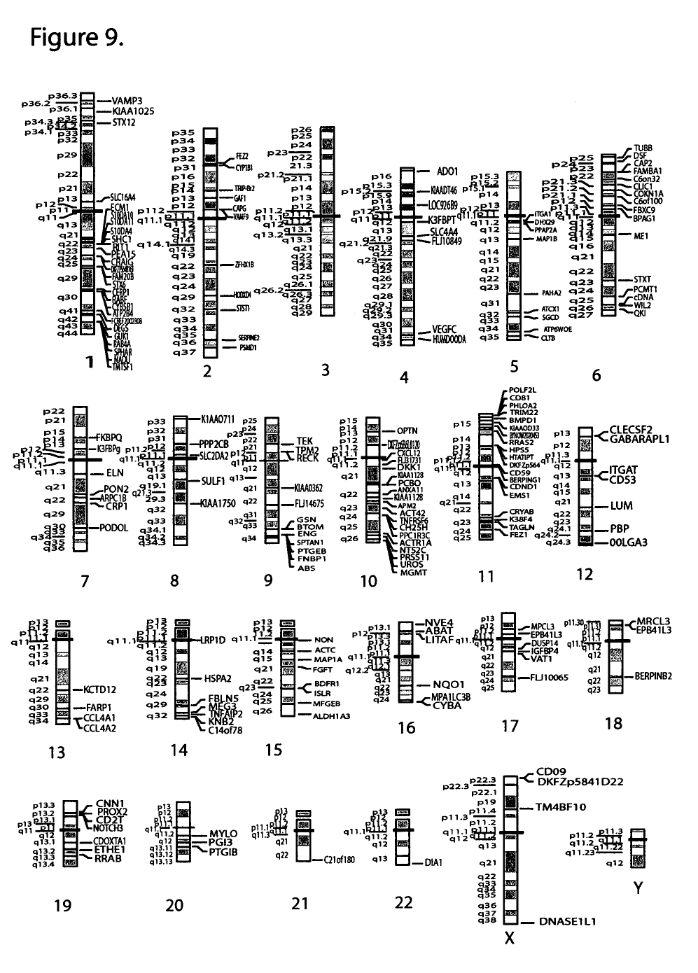

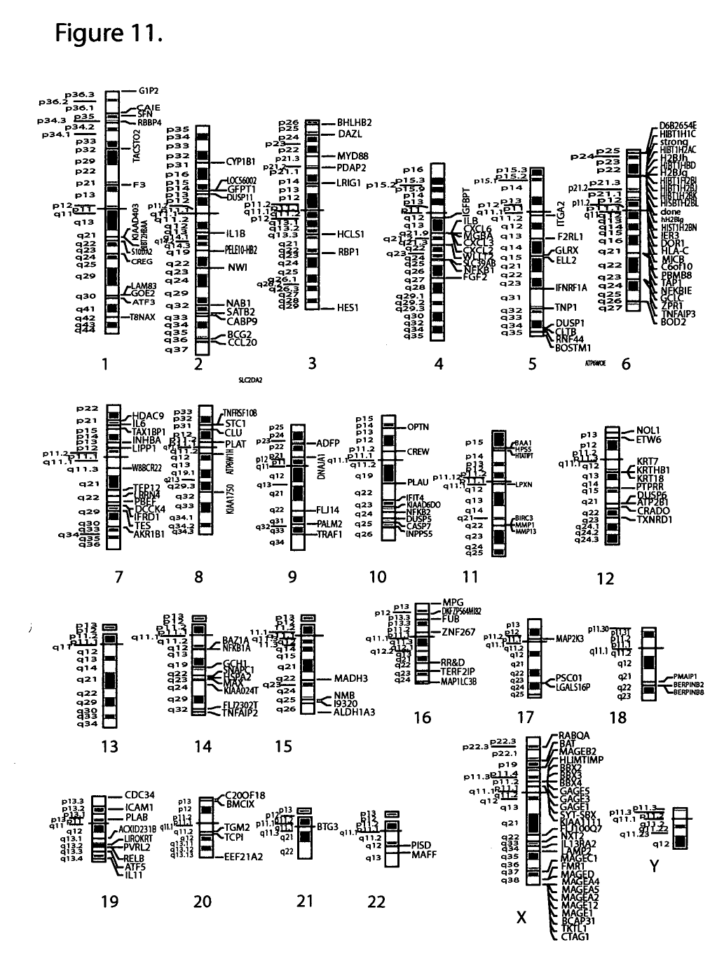

In the analysis there were identified several pathways with changes in gene expression, including the interferon signaling pathway, the cell cycle pathway, and genes for proteins in the cytoskeleton, that were differentially expressed after the immortalization in LFS cells. Fourteen genes were consistently epigenetically regulated during immortalization in all of the immortal cell lines studied, namely CREG, CYP1B1, IGFBPrP1, CLTB, KIAA1750, FLJ14675, OPTN, HPS5, HTATIP2, HSPA2, TNFAIP2, ALDH1A3, MAP1LC3B, and SERPINB2. A significant number of the epigenetically regulated genes, in each of the four immortal LFS cell lines, are in the IFN pathway. The involvement of the IFN pathway in cellular senescence and tumorigenesis is supported by the fact that a number of IFN induced proteins have tumor suppression activity when overexpressed in tumor cells. These proteins include double stranded RNA activated protein kinase (PKR), activated RNaseL, and the 200 gene family (Pitha 2000). Further, genes with expression that decreased during immortalization and increased after 5-aza-dC treatment, in common to all four immortal LFS cell lines, cluster on chromosome 4q12-q27, 6p22, 6p21.3, 7, 14,19 and X (FIGS. 9 and 11).

Immortalization is one of the necessary, multiple steps of tumorigenesis. Normal mammalian somatic cells can only divide a limited number of times in vitro. The maximum number of divisions is called the “Hayflick limit” (Hayflick L. et al., 1961). After that point the cells leave the cell cycle but remain metabolically active. This non-proliferative state is referred to as cellular senescence. Cells undergo a series of biochemical and morphological changes at senescence. Typical characteristics of senescing cells include large, flat morphology, a high frequency of nuclear abnormalities and positive staining for β-galactosidase activity specifically at pH 6.0. Senescence can be induced by a demethylation agent 5-aza-2′-deoxycytidine (5-aza-CdR) (Vogt M et. al, 1998). The counting mechanism for intrinsic replicative lifespan appears to be the shortening of telomeres with each cell division cycle (Counter, C. M. et al, 1992).

Abnormal genetic changes or expression of viral oncoproteins in cells can prolong the division cycle beyond the Hayflick limit (Hayflick L. et al., 1961). The inactivation of p53 and pRb precedes the activation of telomere maintenance mechanism. The disruption of p16^{INK4a} pathway creates a permissive environment for telomerase activation. After additional 20-30 population doublings, cells enter a state, which is referred to as crisis. At crisis, the cells continue to proliferate but have high rate of apoptosis. The expression of human telomerase reverse transcriptase (hTERT) is one of the telomere maintenance mechanisms that allow cells bypass senescence and expand the proliferative life span. The total cell number does not increase. After inactivation of p53 and pRb with DNA viral oncogenes, cells escape crisis and finally become immortalized at a low frequency (˜1 in 10⁷).

In addition to p53, pRb, p16^{INK4a} (Vogt M et. al, 1998) and the genes required for telomere maintenance, some other genes can also involve in immortalization. The observation that not all cancers have mutated p53 suggests the upstream genes of p53 can prevent its normal function. Similarly, other genes involved in the pRb/p16^{INK4a} pathway can substitute the abnormalities of these genes. They are also candidate tumor suppressor genes involved in immortalization (Bryan, T. M. et al., 1995, Kaul, S. C. et al, 1994).

Mortalin is another important gene in cellular senescence and immortalization. The cytosolic mortalin is a marker of the mortal phenotype, however, the perinuclear mortalin can have a role in tumorigenesis (Kaul, S. C. et al, 1994, Wadhwa, R. et al., 1994).

The greatest single risk factor for the development of cancer in mammals is aging. The incidence of cancer increases with age, beginning at about the mid-life span. In general, the rate at which cancer develops is proportional to the rate of aging. For example, mice develop cancer after about a year and a half of age roughly the midpoint in their life span, and humans develop cancer after 50 years, or half way through their life span. By contrast, other age-related diseases, such as Alzheimer's disease, are not believed to develop in short-lived mammals. Both cancer and other age related diseases are final results of a series of small, gradual changes at genetic level. Normal metabolism generates toxins as an inherent side effect. These toxins cause DNA damage, of which a small proportion is unrepaired by endogenous DNA repair mechanisms, and thus mutations accumulate. As DNA damage results in age-related degeneration, interventions must be designed to address molecular targets of aging. Somatic cells respond to these events by exiting the cell cycle and entering senescence, a metabolically active yet quiescent state. Bypassing senescence, commonly known as immortalization, has provided a relevant model for human aging at the cellular level. At the same time, bypassing cellular senescence is one of the necessary, multiple steps of tumorigenesis. Thus the phenomenon of immortalization is crucial to the understanding of both age related illnesses and cancer. By detecting the molecular targets involved in immortalization, one can determine proper targets of cancer prior to the existence of a tumor.

Additionally, as disclosed in Esteller et al., the changes of 16 promoter hypermethylation regulated genes have been examined in over 600 primary tumor samples representing 15 major tumor types (Esteller et al (2001). Their results showed that although some of the gene changes are shared among different tumors, however, 70-90% tumor types do have a unique profile of three to four hypermethylation gene markers. In furtherance of the data disclosed in the Esteller et al. reference, the present invention provides that the promoter region hypermethylation is a molecular marker system for the early diagnosis of major forms of human cancer. Compared to genetic analysis, detection of promoter methylation offers many advantages: a) promoter methylation occurs over the same region within an individual gene, however, other DNA alterations such as mutations often vary over a wide region in the gene; b) promoter hypermethylation offers a positive signal against the background of normal DNA which is easier to detect comparing with the deletion mutation; c) the degree of transcription repression is dependent upon the density of methylation within the promoter region (Hsieh et al (1994); Vertino et al (1996); Graff et al (1997). Thus, the detection of methylation markers can be quantitative and qualitative with the aid of sensitive PCR strategies (Galm et al (2002); Herman, J. G. et al., 1996). Another key feature of methylation is its operational reversibility. Demethylation agents such as 5-azacytidine have already been used as chemotherapeutic agents. The identification of hypermethylation in gene promoters is not only a good molecular marker system for early tumor diagnosis, but also can be a desirable target for gene reactivation.

Although IFN signaling pathways have been reported to be activated by the treatment of methylation inhibitor 5-aza-CdR in bladder and colon cancer cells, the IFN signaling pathway was not previously found to be activated with 5-aza-CdR in an immortal fibroblast preneoplastic cell line. The present invention provides that genes in IFN signaling pathway can be tumor suppressor genes, early genetic or epigenetic events involved in the progression of cells to immortalization and then cancer. The functional study on the biological function of IFN pathway genes in immortalization reveals the mechanism of how cancer cells escape the defense of IFN immune system. As functional genes i.e. candidate tumor suppressor genes in immortalization, these genes can serve as useful diagnostic markers in serum DNA assays or as therapeutic targets. The senescence initiating events leading to genomic instability and telomere stabilization are loss of checkpoint proteins such as p53, p21^{CIP1/WAF1} and p16^{INK4A}. Gene profiling revealed 149 upregulated genes and 187 downregulated genes of which 14 were epigenetically downregulated in all four immortal LFS cell lines. In addition, several common pathways were involved in immortalization including the interferon pathway, genes involved in proliferation and cell cycle control, and the genes for cytoskeletal proteins.

It is known that the immune system becomes less active during aging. The cellular response to interferon-gamma (IFN-gamma), the expression at the cell surface of the MHC class II gene IA complex product and the levels of IA-beta were decreased in aged macrophages (Herrero C et al, 2002). Moreover, the transcription of IFN regulated genes is impaired in aged macrophages. The impaired immune response associated with cellular senescence of immune cells. Indeed certain polymorphisms in IFN-gamma are associated with longevity (Lio 0 et al, 2002). The presence of the +874A allele, known to be associated with low IFN-production, allows extended longevity, possibly due to pro-inflammatory status during aging that might be detrimental for successful aging. The allele was significantly increased in female but not male centenarians seems indicating that a gender variable can be important in the biology of the aging process. It is clear that the IFN pathway is a factor in the aging process.

The markers that are identified by the method of the present invention can then be used for treatment of disease. For example, in cancer, the molecular marker can be suppressed to prevent proliferation of cancerous cells using gene therapy techniques known to those of skill in the art. Alternatively, in aging, the marker can be enhanced to limit the number of cells that die as a normal result of the aging process using gene therapy techniques known to those of skill in the art.

In order to determine which molecular markers are markers of cancer and aging, the microarrays must be analyzed. Preferably, the arrays are analyzed based either on fold change or via a noise sampling method (ANOVA). The fold change method is used to select the genes with at least a twofold change in expression. This is done using the Affymetrix Data Mining Tool (DMT), version 3,N-fold method (Affymetrix, Santa Clara, Calif., USA). For the control versus experiment comparisons, all possible pairings between the two controls and the two experiments are considered. ANOVA analysis (Kerr et al., 2000) can be used to isolate and eliminate the effects of within-slide and interslide variability and other sources of noise in the microarray. The effects of differential dye incorporation can also be eliminated by performing an exponential normalization (Houts, 2000) and/or a piece-wise linear normalization of the data obtained in the first round. The exponential normalization can be done by calculating the log ratio of all spots (excluding control spots or spots flagged for bad quality) and fitting an exponential decay to the log (Cy3/Cy5) vs. log (Cy5) curve. The curve fitted is of the form:
y=a+b^{(-cx)}

where a, b and c are the parameters to be calculated during curve fitting. Once the curve is fitted, the values are normalized by subtracting the fitted log ratio from the observed log ratio.

This normalization has been shown to obtain good results for cDNA microarrays but it relies on the hypothesis that the dye effect can be described by an exponential curve. The piece-wise linear normalization can be done by dividing the range of measured expression values into small intervals, calculating a curve of average expression values for each such interval and correcting that curve using piece-wise linear functions.

All the gene expression data on HGU95Av2 were processed as previously described and used for the hierarchical clustering analysis implemented in GeneSight, version 3.2.6 (Biodiscovery, Los Angeles, Calif.). Euclidean distance was used for measuring similarities between two genes or samples, and complete linkage was used for clustering. For each of the four immortal LFS cell lines there were two comparisons, immortal cells versus precrisis cells, and 5-aza-dC treated immortal cells versus untreated immortal cells. Two-sided hierarchical analysis was carried out to determine the similarities of the four immortal LFS cell lines across the whole gene expression data.

Multidimensional scaling is an alternative way to present the data in low dimension space. Multidimensional scaling analysis was performed using BRB-Array Tools version 3.2 beta to plot the data in three dimensions. The same comparisons and parameters used for hierarchical clustering were also used for multidimensional scaling analysis.

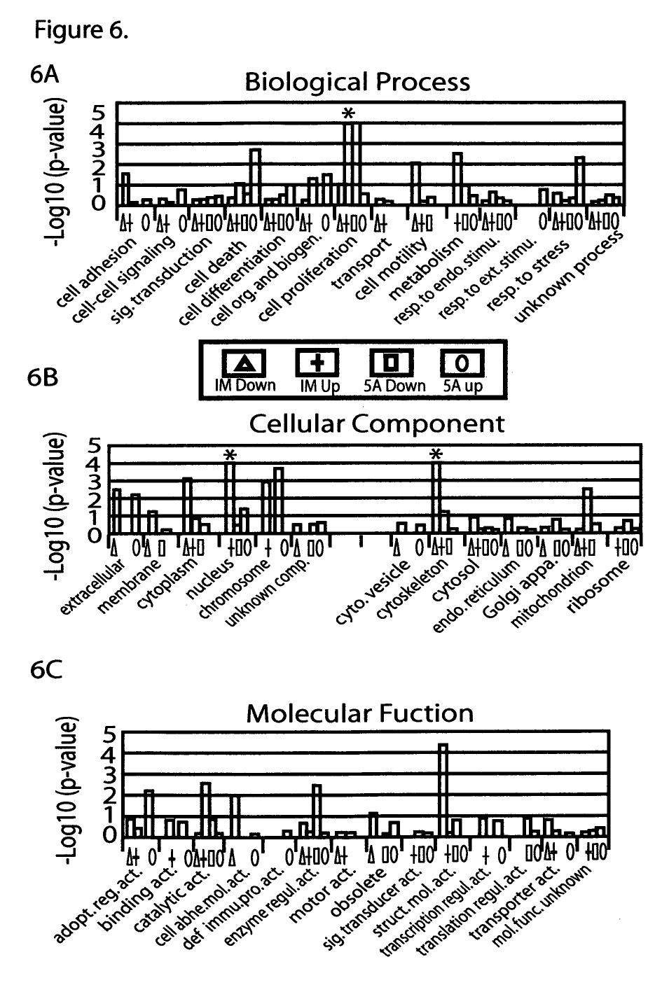

Then, GoMiner (version 122) (Zeeberg et al. 2003) was used to annotate the gene expression data with GO categories. The entire HGU95Av2 GeneChip® probe set was the reference. Four experiment genes lists were analyzed: genes that were up- and downregulated during immortalization in all four immortal LFS cell lines (A and B in Table 7), and genes that were up- and downregulated after 5-aza-dC treatment in all four immortal LFS cell lines (C and D in Table 7). The probes from the lists were first converted to unique gene symbols using NetAffx, the Affymetrix online database (Build # 166) (Liu et al. 2003), and then the unique list of gene symbols were analyzed by GoMiner. The 8,487 unique gene symbols on the HGU95Av2 GeneChip® were linked to 6,020 GO categories. The one-sided Fisher's exact test p-values calculated by GoMiner were used to evaluate the statistical significance of changes for a GO category. The p-values for the first layer GO categories were converted to −log₁₀(p-value) and graphed (FIG. 6).

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

Standard methods in immunology known in the art and not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W. H. Freeman and Co., New York (1980).

Gene therapy, as used herein, refers to the transfer of genetic material (e.g. DNA or RNA) of interest into a host to treat or prevent a genetic or acquired disease or condition phenotype. The genetic material of interest encodes a product (e.g., protein, polypeptide, peptide, functional RNA, antisense) whose production in vivo is desired. For example, the genetic material of interest can encode a hormone, receptor, enzyme, polypeptide or peptide of therapeutic value. Alternatively, the genetic material of interest can encode a suicide gene. For a review, see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, which is within the recipient. In an alternative embodiment, if the host gene is defective, the gene is repaired in situ [Culver, 1998]. These genetically altered cells have been shown to express the transfected genetic material in situ.

The gene expression vehicle is capable of delivery/transfer of heterologous nucleic acid into a host cell. The expression vehicle can include elements to control targeting, expression and transcription of the nucleic acid in a cell selective manner as is known in the art. Often the 5′UTR and/or 3′UTR of the gene can be replaced by the 5′UTR and/or 3′UTR of the expression vehicle. Therefore as used herein the expression vehicle can, as needed, not include the 5′UTR and/or 3′UTR of the actual gene to be transferred and only include the specific amino acid coding region.

The expression vehicle can include a promotor for controlling transcription of the heterologous material and can be either a constitutive or inducible promotor to allow selective transcription. Enhancers that can be required to obtain necessary transcription levels can optionally be included. Enhancers are generally any non-translated DNA sequence that works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor that includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention can depend on desired cell type to be targeted and can be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles that are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector can depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors can circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

The above discussion provides a factual basis for the use of microarrays for detecting molecular markers of cancer and aging as disclosed above. The methods used with a utility of the present invention can be shown by the following non-limiting examples and accompanying figures.

### EXAMPLES

### Example 1

Abrogating cellular senescence is a necessary step in the formation of a cancer cell. Promoter hypermethylation is an epigenetic mechanism of gene regulation known to silence gene expression in carcinogenesis. Treatment of spontaneously immortal Li-Fraumeni fibroblasts with 5-aza-2′-deoxycytidine (5AZA-dC), an inhibitor of DNA methyltransferase (DNMT), induces a senescence-like state. Microarrays containing 12,558 genes were used to determine the gene expression profile associated with cellular immortalization and also regulated by 5AZA-dC. Remarkably, among 85 genes with methylation-dependent downregulation (silencing) after immortalization, 39 (46%) are regulated during an interferon signaling known growth-suppressive pathway. The data included herein indicates that gene silencing can be associated with an early event in carcinogenesis, cellular immortalization.

Immortalization is one of the necessary, multiple steps of tumorigenesis. Normal mammalian somatic cells can only divide a limited number of times in vitro. The maximum number of divisions is called the ‘Hayflick limit’ (Hayflick, 1976). This non-proliferative state is also referred to as replicative cellular senescence. Typical characteristics of senescing cells include a large, flat morphology, a high frequency of nuclear abnormalities, and positive staining for β-galactosidase activity specifically at pH 6.0. The counting mechanism for the intrinsic replicative lifespan appears to be the shortening of telomeres with each cell division cycle (Huschtscha and Holliday, 1983). The phenotype of senescence is a dominant trait, and the genes associated with it fall into four complementation groups (Pereira-Smith and Smith, 1983).

Human cells can be immortalized through the transduction of viral and cellular oncogenes (Graham et al., 1977; Huschtscha and Holliday, 1983), various human oncogenes such as c-myc (Gutman and Wasylyk, 1991), or in some rare cases spontaneously (Bischoff et al., 1990; Rogan et al., 1995; Shay et al., 1995). These mechanisms of immortalization result in abrogation of p53 and pRB/p16^{ink4}-mediated terminal proliferation arrest and the activation of a telomere maintenance mechanism (Rogan et al., 1995; Duncan et al., 2000). The activation of human telomerase reverse transcriptase (hTERT) expression is one of the telomere maintenance mechanisms that allow cells to bypass senescence. Certain immortalized human cell lines (Bryan et al., 1995) and some tumors (Bryan et al., 1997) maintain their telomeres in the absence of detectable telomerase activity by a mechanism, referred to as alternative lengthening of telomeres (ALT), that can involve telomere-telomere recombination (Dunham et al., 2000).

Senescence can also be induced in immortal cells by a DNA methyltransferase (DNMT) inhibitor, 5-aza-2′-deoxycytidine (5AZA-dC) (Vogt et al., 1998), implying that replicative senescence can result from epigenetic changes in gene expression (Herman and Baylin, 2000; Newell-Price et al., 2000; Baylin et al., 2001). Genes regulated by DNA methylation usually contain upstream regulatory regions and immediate downstream sequences enriched in CpG dinucleotides (CpG islands). Cytidine residues within CpG islands are methylated by DNMT that can recruit histone deacetylases resulting in the formation of condensed chromatin structures containing hypoacetylated histones. Hypomethylation of CpG islands in oncogenes and hypermethylation of tumor-suppressor genes are important regulatory mechanisms in tumor initiation and progression of cancer (Vogt et al., 1998; Baylin et al., 2001).

Li-Fraumeni syndrome (LFS) is a familial cancer syndrome that is characterized by multiple primary tumors including soft-tissue sarcomas, osteosarcomas, breast carcinomas, brain tumors, leukemias, adrenal-cortical carcinomas, to a lesser extent melanoma and carcinomas of the lung, pancreas, and prostate. Heterozygous germlne p53 mutations were found in 75% of families having LFS (Malkin et al., 1990; Malkin, 1994). Fibroblast cell lines established from individuals with LFS develop changes in morphology, chromosomal abnormalities, and spontaneously form immortal cell lines (Hayflick, 1976; Bischoff et al., 1990; Malkin et al., 1990). Vogt et al. (1998) demonstrated that the treatment of immortal LFS fibroblasts with 5AZA-dC results in arrest of growth of the fibroblasts and development of a senescent phenotype. Repression of gene expression because of methylation-dependent silencing occurs upon cellular immortalization and a significant proportion of these genes are regulated in the interferon (IFN) pathway. Silencing of this growth-suppressive pathway can be an important early event in the development of cancer, specifically associated with immortalization.

Materials and Methods

Cell Culture and p53 Genotyping

The MDAH041 (p53 frameshift mutation) cell line was derived from primary fibroblasts obtained by skin biopsy from patients with LFS. Characterization and immortalization of these cells was performed by Bischoff et al. (1990). All cells were grown in modified Eagles medium (MEM, Gibco BRL, MD, USA) with 10% fetal calf serum and antibiotics. The CRL1502 cell line was derived from primary fibroblasts obtained by skin biopsy from a normal donor (ATCC 1502, Rockville, Md., USA). The region containing the frameshift mutation in gene encoding p53 from LP preimmortal and HP immortal cells was sequenced to confirm the heterozygosity in LP preimmortal MDAH041 cells. Treatment of cells with 5AZA-dC Fibroblast cell cultures were seeded 3×10⁵ per plate in MEM medium with 10% fetal calf serum and antibiotics. Cell cultures were treated with 1 μM 5AZA-dC on days 1, 3, and 5 each time with a full media change. After day 6, the cells were returned to regular medium without 5AZA-dC. Total RNA preparation was performed on day 8.

RNA Isolation and the Affymetrix Microarray Assays

The cells were grown to 80% confluence, the medium was changed, and after 16 hours the cells were washed with PBS, trypsinized, and pelleted at 300 g for 5 minutes. Total RNA was isolated using RNeasy kit (Qiagen Inc., Valencia, Calif., USA). 1.5×10⁷ cells yielded 200 μg total RNA. The RNA targets (biotin-labelled RNA fragments) were synthesized from 5 μg of total RNA by first synthesizing double-stranded cDNA followed by standard Affymetrix protocols (Affymetrix, Santa Clara, Calif., USA).

Quantitation of Gene Expression by Q-RT-PCR

Total RNA (1 μg was reverse transcribed into cDNA using Superscript II (Life Technologies, Gaithersburg, Md., USA). All methods for reactions were performed as recommended by the manufacturer. The ABI 5700 Sequence Detection System was used for Q-RT-PCR. The protocols and analysis of data are identical to that of the ABI 7700 Sequence Detection System (ABISYBR). All methods for reactions and quantitation were performed as recommended by the manufacturer. An extensive explanation and derivation of the calculations involved can be found in the ABI User Bulletin× and also in the manual accompanying the SYBR Green PCR core kit. Primers used in Q-RT-PCR are shown in Table 11.

Analysis of Microarray Data

Microarray experiments were performed using the Affymetrix HG-U95A chip containing 12,558 probes. Two RNA preparations from immortal cells (HP) were compared with two RNA preparations from preimmortal cells (LP). In addition, two RNA preparations from immortal cells (HP) were compared with three total RNA preparations from immortal cells treated with 5AZA-dC using the HG-U95A chips.

Two analysis methods were used to select differentially regulated genes: fold change and noise sampling method (ANOVA). The fold change method was used to select the genes with at least a twofold change in expression. This was done using the Affymetrix Data Mining Tool (DMT), version 3,N -fold method (Affymetrix, Santa Clara, Calif., USA). For the control versus experiment comparisons, all possible pairings between the two controls and the two experiments were considered.

The noise sampling method is a variation of the ANOVA model proposed by Kerr and Churchill (Kerr et al., 2000; Draghici, 2002). The noise sampling method was implemented in GeneSight, version 3.2.21 (Biodiscovery, Los Angeles, Calif., USA). In order to apply the noise sampling method, the intensities obtained from each chip, were normalized by dividing by the mean intensity. Four ratios were formed by taking all possible combinations of experiments and controls. Genes differentially regulated with a 99.99% confidence (P ¼ 0.0001) were detected.

CPG Island Analysis

First, the genome sequence of each IFN-regulated RNA from UCSD Genome Browser (http:Hlgenome.ucsc.eduI) was retrieved. Then, the CpG islands were tested within an interval of 500 to 200 bp around the transcription starting site (TSS) using CpGPlot program (http://www.ebi.ac.uk/emboss/cpgplot/). The discrimination for CpG islands is based on the formal definition of CpG islands (Gardiner-Garden and Frommer, 1987)(length is over 200 bp, G+C content is greater than 50%, statistical expectation is greater than 0.6).

Results

Chances in Gene Expression After Immortalization

Preimmortal (PD 11) and immortal (PD 212) fibroblast cells (MDAH041 cell line) from an LFS patient were employed to analyze the changes in gene expression during cellular immortalization. Total RNA was isolated from these cells and probes were synthesized for hybridization to microarrays, Affymetrix HGU95Av2 GeneChips. The genes were selected using two different methods: (i) the classical method of selecting the genes with at least a predetermined fold change and (ii) an ANOVA-based noise sampling selection method (Draghici, 2002). All the four possible pairings between preimmortal vs immortal cell gene expression comparisons were performed using independent cellular RNAs prepared from these cells. The fold change method was used to select the genes with twofold or greater change in gene expression. There were 169 upregulated and 450 down-regulated genes satisfying this condition (Table 1). The noise-sampling selection method is based on ANOVA (Kerr et al., 2000) and uses replicate measurements to estimate an empirical distribution of the noise. Given this distribution and a chosen confidence level, one can establish which genes are differentially regulated beyond the influence of the noise. The method identified 76 upregulated and 217 downregulated genes.

The two methods are in some sense complementary. The noise-sampling method selects those genes that have reproducible changes higher than the noise threshold at some confidence level, whereas the N -fold method selects those genes that have a minimal fold change that can be confirmed with other assays such as quantitative real time PCR (Q-RT-PCR). The intersection of the subsets of genes reported as differentially regulated by both methods identified 59 upregulated genes and 192 downregulated genes after immortalization (Table 1). Using a representative set of the genes satisfying both conditions (for both downregulated and upregulated genes), the microarray data were confirmed using Q-RT .PCR (Table 2). Comparison of the levels of gene expression after immortalization obtained by using both microarray hybridization and Q-RT-PCR revealed outstanding accuracy of the data. Since Q-RT-PCR data can cover a larger range of expression levels, the data obtained using microarrays and Q-RT-.PCR differed quantitatively.

Effect of 5AZA-dC Gene Expression in Immortal LFS Fibroblasts

As was first shown by Fairweather et al. (1987), in vitro lifespan of normal human fibroblasts could be shortened by exposure of the cells to the demethylating agent 5AZA-dC. In agreement with this, Vogt et al. (1998) have shown that treatment of LFS immortal fibroblasts with 5AZA-dC results in growth arrest and senescence. Thus, there is a possibility that development of immortalization is related to methylation-induced silencing of gene expression. To address this issue, the immortal cells (MDAH041 high passage cell culture) were treated with 5AZA-dC to induce gene demethylation. Treated MDAH041 cells had flat morphology, contained lipofuscin granules, and showed senescence associated β-galactosidase activity at pH 6, typical for the senescent cells (Dimri et al., 1995). Total RNA was prepared from MDAH041, high-passage (HP) treated or untreated with 5AZA-dC,and used to prepare probes for the microarray hybridizations. Affymetrix HGU95Av2 GeneChips were again used and the data were analyzed as described above for the comparison of preimmortal and immortal MDAH041 cells. The comparison of treated and untreated HP cells identified 48 5AZA-dC upregulated and 190 5AZA-dC downregulated genes with at least a twofold change and 150 upregulated and 328 down-regulated genes selected by ANOVA (Table 1). There were 81 upregulated genes and only one downregulated gene that satisfied both conditions (P<α and fold change >2 (Table 1). A sampling of genes covering a range of gene expression changes was chosen and confirmed using Q-RT-PCR (Table 3).

It was then determined whether changes in gene expression using Q-RT-PCR after 5AZA-dC treatment were specific to cells undergoing senescence by comparing gene expression changes induced by 5AZA-dC treatment in normal mortal human fibroblasts with those in the immortal MDAH041 cells. The expression levels of 15 of these genes were analyzed in preimmortal low-passage (LP) MDAH041 and normal mortal fibroblast cells (CRL-1502) untreated or treated with 5AZA-dC using Q-RT-PCR (Table 4). The vast majority of the 5AZA-dC-dependent changes in expression found in the immortal MDAH041 cells were not induced by 5AZA-dC treatment of the normal human fibroblasts or preimmortal MDAH041 LFS fibroblasts. The exception, IFN-inducible p27, is found in a known imprinted region on chromosome 14q32 and its induction by 5AZA-dC in all cells therefore was not surprising. In summary, while treatment with 5AZA-dC strongly induces expression of many genes silenced in immortal cells, the expression levels of the same genes were not significantly affected by 5AZA-dC treatment of mortal fibroblasts.

Genes Downregulated After Immortalization and Silenced by Gene Methylation

Since 5AZA-dC-induced gene expression results in the reversal of immortal phenotype and the induction of a senescent-like state, it was investigated whether inhibition of DNMT by 5AZA-dC upregulates genes repressed after immortalization. Table 5 shows the list of 85 genes selected by either or both selection methods as silenced after immortalization due to methylation. Interestingly, when the ‘reverse’ identification of genes was attempted (i.e. genes, both upregulated after immortalization but repressed by 5AZA-dC), no common genes were identified using the dual selection method approach (Table 1,comparison of A and C). In view of the fact that the numbers of genes identified in these comparisons (comparisons B and D (85 genes), and A and C (three genes)) were so vastly different, these suggested that methylation-dependent gene silencing is mechanistically significant to the process of immortalization. Microarray analysis of MDAH041 cells containing a tetracycline-modulated p53 gene revealed that none of these 85 genes were regulated by p53 in these cells. Analysis of the functional annotations of the genes downregulated in immortalization (Table 5), because of methylation-dependent silencing, revealed that a significant fraction, 39 out of 85 genes, are known to be regulated by the IFN pathway, with 19 of the 39 genes containing CpG islands identified using CpGPlot software (Table 6).

Hierarchical Clustering

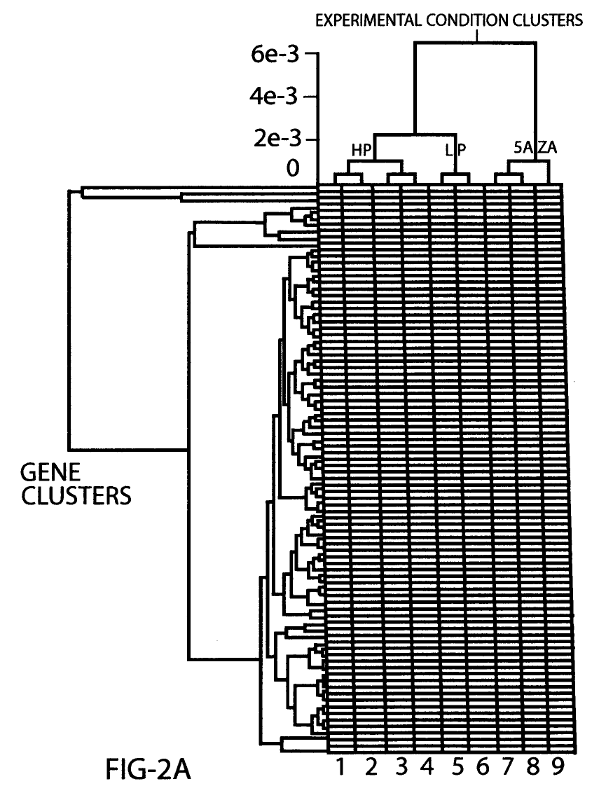

The hierarchical map of the silenced gene expression set and two subsets of genes (identified by both software methods) that are repressed after immortalization by methylation-dependent silencing is shown in FIGS. 2a, b. In these figures, the height of each bridge between members of a cluster is proportional to the average squared distance of each leaf in the subtree from that subtree's centroid (or mean). These data indicate that the level of expression of the same set of genes that are downregulated during immortalization is also stimulated by 5AZA-dC-induced DNA demethylation. Interestingly, the approach showed that the total pattern of gene expression (12,558 genes) in preimmortal MDAH041 cells is similar to the 5AZA-dC-treated immortal MDAH041 cells as compared to the untreated immortal cells. In FIG. 2a, the set of 5 genes silenced by methylation show a pattern of low expression in the immortal fibroblasts (indicated by the green color) and higher expression in the preimmortal MDAH041 cells and in the 5AZA-dC-treated immortal cells (indicated by the red color). FIG. 2b similarly shows the pattern of gene expression in the group of 30 genes selected by 99.99% confidence and a greater than twofold change in expression.

Discussion

The indefinite lifespan necessary for the formation of a cancer cell appears to be a complex genetic trait with four complementation groups of recessive genes (Pereira-Smith and Smith, 1983, 1988; Berube et al., 1998). Since treatment of spontaneously immortalized Li-Fraumeni cells, MDAH041, with the DNMT inhibitor, 5AZA-dC, results in a replicative senescent state (Baylin et al., 2001), epigenetic control of immortalization needed to be considered in these cells. Affymetrix microarrays were employed to profile gene expression changes associated with immortalization and determined which of those genes were also regulated by DNA demethylation. Genes downregulated after immortalization (493 genes) fit the pattern of recessive senescence genes predicted by the somatic cell genetics experiments (Pereira-Smith and Smith, 1988). Consistent with this hypothesis, it was reasoned that those in common with the 190 genes upregulated after the 5AZA-dC treatment would focus the gene set on those involved in replicative senescence. This gene set included a total of 85 genes from those available on the microarrays used. One of these genes is known to be maternally imprinted in the Prader-Willi Syndrome, NDN (Jay et al., 1997) (Table 5). The protein encoded by this gene, Necdin, is a growth suppressor expressed in postmitotic neurons of the brain (Nakada et al., 1998). The RNA is silenced during immortalization and activated by 5AZA-dC treatment of the immortal MDAH041 cells but not normal fibroblasts or preimmortal MDAH041 (Table 4). Interestingly, this gene was found to undergo loss of heterozygosity in the MDAH041 immortal cells.

Downregulation in immortal MDAH041 cells of some genes (collagenase, cathepsin O, uPA) was observed that have been detected by others as upregulated genes during replicative senescence in dermal fibroblasts (Shelton et al., 1999). Downregulation of DOC1, IGFBP4 and IGFBP6 was also observed in immortal cells that is correlated with the published data before of Schwarze et al. (2002) who found upregulation of DOC1 and IGFBP3 in human prostate epithelial cells when passaged to senescence.

Remarkably, 39 of these 85 genes were also known to be regulated in the IFN pathway and represent candidate regulatory genes in cellular immortalization. These data are in agreement with others who observed 5AZA-dC upregulation of IFN pathway genes in colon tumor cells (Karpf et al., 1999) and human bladder cancer cells (Liang et al., 2002). To calculate the significance of this observation, the UniGene clusters were used in order to eliminate overcounting genes with several accession numbers and/or Affymetrix probes. Currently, the 12,558 probes on the array correspond to 8628 Unigene clusters. Among these, there are 137 genes, or 0.015%, known to be IFN-regulated. Thus, a list of 85 random genes contains about 85 0.015% or approximately zero INF-regulated genes due to random chance. In fact, the list of 85 genes silenced in immortalization contained 39 IFN-regulated genes. The probability of this happening by chance is approximately 10 47 which shows that the silencing of the IFN-pathway genes is highly significant to the mechanism of cellular immortalization.

Some IFN-regulated genes have previously been shown to be silenced by DNA methylation and reactivated by 5AZA-dC treatment (Liang et al., 2002). Consistent with this observation and the growth-inhibitory effect of IFNs, 5AZA-dC treatment has been shown to inhibit the growth of human tumor cell lines (Bender et al., 1998) and the data indicate that gene silencing can be an early event in cancer development. The IFN-regulated RNaseL gene is known to inhibit cell proliferation and induce apoptosis through the IFN-regulated (2′-5′) oligoadenylate synthetase pathway. RNaseL is a candidate tumor-suppressor gene that has been shown to be mutated in the germ line of hereditary prostate cancer patients (Carpten et al., 2002). This candidate tumor-suppressor gene, RNaseL, is activated by (2′-5′) oligoadenylate synthetase proteins and therefore it is noteworthy that in MDAH041 cells, three out of four of the isoforms of the (2′-5′) oligoadenylate synthetase are downregulated after immortalization because of methylation-dependent silencing (Table 6). In addition, IRF-1 has been shown to be a tumor-suppressor gene in human leukemias (Harada et al., 1993; Willman et al., 1993). The double-stranded RNA-activated protein kinase (PKR) has been shown to induce apoptosis, implying that its inactivation would be a procarcinogenic event (Jagus et al., 1999). The IFN-inducible proteins of the ‘HIN-200 gene family’ have been demonstrated to be growth inhibitory, have antitumor activity (Wen et al., 2001; Xin et al., 2001), and are able to bind to the Rb1 and p53 tumor-suppressor proteins (Choubey and Lengyel, 1995). One of the three members of this gene family, AIM2, is downregulated in MDAH041 cells and silenced by methylation (Table 6). AIM2 functions as a tumor suppressor for a melanoma cell line (DeYoung et al., 1997) and a T-cell tumor antigen in neuroecto-dermal tumors, as well as breast, ovarian, and colon carcinomas (Harada et al., 2001). The AIM2 gene contains a site of microsatellite instability (MSI) that results in gene inactivation in 47% of colorectal tumors analyzed with high MSI (Mori et al., 2001). Interestingly, p202, a member of the murine ‘200 gene family’, is a negative regulator of p53 whose gene expression is controlled by p53 as well (D'Souza et al., 2001).

MDAH041 LFS cells contain significant telomerase activity after immortalization (Gollahon et al., 1998). Although in microarray analysis, the hTERT gene for the protein of enzymatic subunit of telomerase was not significantly upregulated after immortalization of MDAH041 cells, 1.6-fold, using Q-RT .PCR that there was a significant increase in hTERT expression, 486-fold (Tables 2 and 7). This is consistent with the experience that genes with low basal expression levels are difficult to quantitate accurately using micro-arrays alone. 5AZA-dC treatment resulted in an additional 17-fold increase in hTERT RNA expression (Table 3). Interestingly, the promoter of the hTERT gene has been shown to be regulated by methylation at CpG islands (Dessain et al., 2000; Bechter et al., 2002). Using CpGPlot, an analysis was performed for the presence of CpG islands in the 39 interferon-regulated genes that were identified. In all, 19 of those genes contained CpG islands (Table 6). A subset of these 19 genes represent the primary inducers of cellular senescence and/or aging.

p16^{INK4a} is one of the tumor-suppressor genes whose expression is repressed by methylation, which permits cells to bypass early mortality checkpoints. Downregulation of p16 mRNA in immortal cells and upregulation by demethylation using RT .PCR was confirmed. When the level of protein expression was tested using Western blots, it was found that p16^{INK4a} protein was much less abundant in immortal cells and upregulated approximately 500-fold by 5AZA-dC treatment. The 5AZA-dC-dependent upregulation of p16^{INK4a} protein in immortal MDAH041 cells was observed by us and by Vogt et al. (1998), who demonstrated that retroviral transduction of a p16^{INK4a} cDNA was able to induce senescence in MDAH041 cells. Although retroviral transduction of a p21 cDNA was also able to induce senescence in MDAH041 cells (Vogt et al., 1998), p21 protein levels were not regulated by 5AZA-dC treatment of immortal MDAH041 cells. It is noteworthy that p21^{cip/waf} was also identified as sdi1 because of its high levels of expression in senescing mortal fibroblasts (Noda et al., 1994) and is regulated transcriptionally by DNMT (Young and Smith, 2001). p21 can also be regulated by STAT1 that is also a major transcriptional effector of the IFN pathway (Agrawal et al., 2002). The level of STAT1 protein is two-fold downregulated after immortalization and 4.7-fold upregulated in immortal cells by 5AZA-dC treatment. Therefore, STAT1 is silenced by methylation in immortal MDAH041 cells (Tables 5 and 6) and can be a key regulator of immortalization by controlling the interferon-regulated gene expression pathway and its growth-suppressive effectors. As these mechanisms become better understood, specific demethylation or deacetylation agents currently in preclinical evaluation and clinical trials in cancer patients can provide another approach to control cancer (Brown and Strathdee, 2002).

### Example 2

An indefinite lifespan or cellular immortalization is a necessary step in the formation of a cancer cell. Promoter hypermethylation is an important epigenetic mechanism of gene regulation in the development of cancer, cellular immortalization and aging. Oligonucleotide microarrays were used to discover the gene expression changes associated with cellular immortalization and compared those changes due to variations in gene expression after inhibiting DNA methylation in immortal fibroblast cells with 5-aza-2′-deoxycytidine. The goal was to identify candidate regulatory genes for immortalization as those regulated under both conditions. Among 84 such regulated genes, 31 genes were identified that are known to be involved in interferon-cytokine/JAK/STAT signaling, which are pathways known to be growth suppressive. These and other pathways of gene expression are thus highlighted as important molecular targets for intervention in cancer and aging.

Cellular Immortalization

Smith et al. in 1998 used cell fusion experiment to group >40 immortal human cell lines into four complementation groups. Cell lines in the same complementation group generated hybrids with unlimited division potential. However, cell lines in different complementary group generated hybrids with a finite number of cell divisions (Pereira-Smith et al (1988). Based on this finding, later research used microcell-mediated chromosome transfer technique to identify involvement of mortality factor on chromosome 4 (MORF4) in cell senescence and immortalization (Leung et al (2001).

DNA Methylation

DNA Methylation as an epigenetic regulation in carcinogenesis gene function can be disrupted through either genetic alternations or epigenetic alternations. Genetic alternations include direct gene mutation or deletion. However, epigenetic alternations indicate the inheritance of aberrant states of gene expression following cell division. DNA methylation is one epigenetic mechanism that modifies the genome via covalent addition of a methyl group to the 5-position of cytosine ring in CpG dinucleotide (Holliday, (1990); Bird (1992); Boyes et al (1991). CpG dinucleotides usually cluster at the 5′-ends of regulatory region of genes and are referred to as CpG islands (Boyes et al (1991). DNA methylation in these CpG islands correlate with transcription silencing of the genes. The transcription repression can partly due to the affected ability of DNA-binding proteins to interact with their cognate cis elements (Jaenisch R. (1997). Methylation also plays a key role in genomic imprinting. The regulation of the imprinted gene expression is assumed to be a kind of competition between sense and antisense transcripts on both parental alleles. The methylation patterns of downstream region of the promoter, e.g. imprint control region (ICR) for Igf2 and differentially methylated region 2 (DMR2) for M6P-Igf2r determine the expression of antisense transcript or sense transcript of the imprinted allele (Barlow et al (1991); Counts et al (1996). The normal methylation status is very important for the maintenance of genome stability and abnormal methylation status can lead to carcinogenesis. Hypomethylation can lead to the aberrant expression of oncogenes (Ming et al (2000); Makos et al (1993) and regional hypermethylation can lead to genetic instability and transcription inhibition of tumor suppressor genes (Makos et al (1993); Magewu et al (1994). The methylated CpG sites in the p53 coding region act as hotspots for somatic mutations and account for 50% and 25% inactivating mutations in colon cancer and general cancers (Greenblatt et al (1994); Baylin et al (2001) as well as most germ line mutations in p53.

Promoter Hypermethylation and Carcinogenesis

Promoter hypermethylation has been indicated to be an early event in tumor progression (Wales et al (1995). The genes whose expression have been repressed by promoter hypermethylation have been suggested to be candidate tumor suppressor genes. Various techniques have been applied to search for epigenetically silenced genes in cancer, including searching in frequent LOH regions for promoter hypermethylation (Costello et al (2000);, restriction landmark genomic scanning (Toyota et al (1999), methylated CpG amplification-restriction digest analysis (Liang et al (2002) and microarray (Peris et al (1999). So far, promoter hypermethylation of numerous genes has been identified and their relation to carcinogenesis has been analyzed. This list includes p16^{INK4a}, p15^{INK4b}, p14^{ARF}, p73, APC, BRCA1, hMLH1, GSTP1, MGMT, COH1, TIMP3, DAPK, E-cadherin, LKB1, hSRBC etc. These genes play an important role in cellular pathways of DNA repair, cell cycle regulation, cell-cell recognition and apoptosis, which are important for regulation of tumor formation and aging. Wild type p16^{INK4a} is a negative regulator of cell cycle. It can bind to cyclin-dependent kinase 4 (cdk4) and cyclin-dependent kinase 6 (cdk6) and prevent their phosphorylation of the retinoblastoma protein. The cell cycle progression through the G1 phase is thus blocked (Belinsky et al (1998). The promoter methylation of p16/NK4a has been studied in a wide range of tumor types (Foster et al (1998). The inactivation of p16/NK4a has been implicated in the immortalization process. (Loughran et al (1996); Brenner et al (1998); Kiyona et al (1998); Counts et al (1995) Besides the genes studied, a broad survey for more genes involved in carcinogenesis is ongoing. Since genetic and epigenetic regulations of gene function are cooperative in carcinogenesis (Baylin et al (2001), genes identified from promoter hypermethylation alone as a candidate tumor suppressor gene should be followed by intensive functional analysis for their biological importance (Malkin et al (1990).

MDAH041 Cell Line

MDAHO41 cells derived from patient with Li-Fraumeni syndrome were used. Li-Fraumeni syndrome is a rare familial dominant inherited cancer syndrome. Approximately 75% of LFS patients carry a germline mutation in the p53 gene (Malkin et al (1990). There is a high frequency of somatic mutation in the remaining wild type allele of p53, which leads to the spontaneous immortalization in LFS fibroblast. The MDAHO41 cell line has a point deletion in the p53 allele and the p53 protein is truncated. In precrisis MDAHO41 cells (population doubling <43), the wild type p53 is present and the cells do not have detectable telomerase activity. In postcrisis MDAHO41 cells, the expression of p53 decreases, due to the loss of the wild type allele of p53 and telomerase activity can be detected (Gollahon et al (1998). It has been reported that the treatment of MDAHO41 cells with 5-aza-CdR results an arrest of growth of fibroblast and senescence-associated β-Galactosidase activity at pH 6 (Vogt M et. al, 1998). In the study, low passage MDAHO41 (precrisis) and high passage MDAHO41 (postcrisis) cells were used to study the changes in gene expression in the cellular progression to immortalization. The high passage MDAHO41 cells were then treated with 5-aza-CdR, trying to detect the genes upregulated by promoter dehypermethylation. The observation that cells with p53 germline mutations can spontaneously immortalize (Bischoff et al (1990); Bischoff et al (1991); and can be transformed into tumor cells by oncogenes.

5-aza-2′-deoxycytidine Treatment of MDAHO41 Cells

Treatment of immortal MDAH041 cells with 5-aza-2′-deoxycytidine results in a senescent-like state (Vogt M et. al, 1998). MDAH041 cells were cultured at 37° C. in 10% humidified CO₂ in DMEM (10% FBS, 500 units/ml penicillin, 100 μg/ml streptomycin. The cells were treated with 1 μM 5-aza-2′-deoxycytidine for 6 days with media changes on days 1,3, and 5.

Immunoblotting of 16^{INK4a} Protein After 5-aza-CdR Treatment

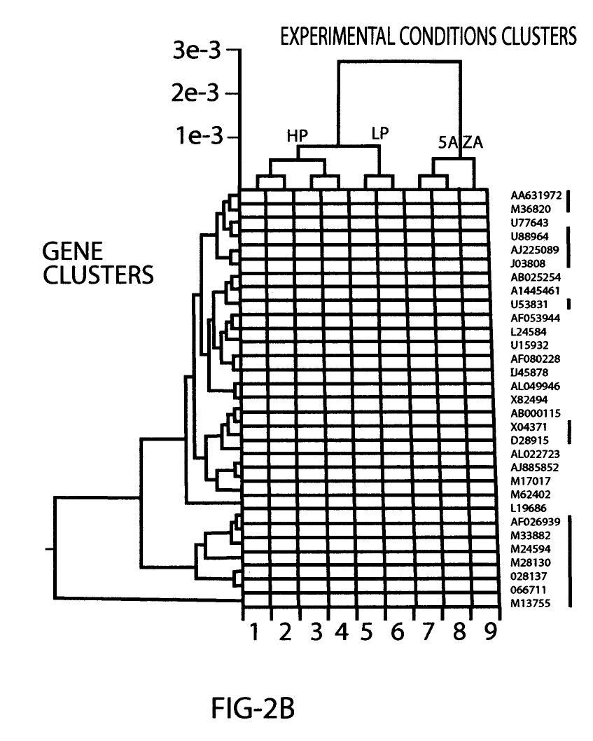

The tumor suppressor p16^{INK4a} protein is known to be regulated by DNA methylation at its promoter and to be able to induce senescence in immortal cells, (Vogt M et. al, 1998). Twenty μg of cell extract was boiled for 5 minutes in sample buffer, electrophoresed on a 15% SDS-polyacrylamide gel, and transferred to nitrocellulose. The blots were blocked with 5% nonfat dry milk and incubated with purified anti-human p16^{INK4a} diluted 1:5,000 at 4° C. overnight. The anti-mouse IgG was incubated with the blot for 1 hour at room temperature. The signal was detected by enhanced chemiluminescence. SAOS2 cells and HT1080 cells served as positive and negative control for p16^{INK4a}, respectively. The expression of the p16^{INK4a} protein was upregulated over 500 fold in the 5-aza-CdR-treated MDAH041 cells, as compared to the expression in the untreated immortal MDAH041 cells (FIG. 3). This is consistent with previously published work that p16^{INK4a} protein is upregulated by 5-aza-CdR-induced DNA demethylation in MDAH041 immortal cells (Vogt M et. al, 1998).

Affymetrix Oligonucleotide Array Analysis of Gene Expression

Affymetrix array was performed on low passage MDAHO41, 5aza-CdR treated and non-treated high passage MDAHO41 cells with three replicates of each in the lab. mRNA were reverse transcribed into cDNAs. DNA chips were performed followed the protocols from Affymetrix (Santa Clara, Calif.). The microarrays were scanned and processed.

Data Analysis

The expression profiles were analyzed with Data Mining Tools of Affymetrix. The expression level of the genes in 5-aza-CdR treated MDAHO41 cells were compared with those of untreated cells. Genes whose expression levels were up regulated >2 fold in 5-aza-CdR treated cells were selected (Table 1). The gene expression levels in high passage MDAH041 cells were compared with those of low passage MDAH041 cells (Table 1). The genes whose expression level were down-regulated >2 folds in high passage immortal cells were selected. The genes whose expression levels are low in untreated high passage, immortal MDAH041 cells but high after 5-aza-CdR treatment were candidate tumor (or growth) suppressor genes whose expression has been repressed by promoter hypermethylation in immortal cells. By intersecting the two groups of genes, 84 genes upregulated by demethylation and downregulation during immortalization were identified, Table 1. The differential expression of many of the genes was confirmed by quantitative RT-PCR, Table 2. After functional annotation of the 84 genes from GeneOntology, it was found that these 84 genes involved in a broad range of pathways including cell-cell signaling, transcription regulation, cellular proliferation, and cell adhesion. By examining these genes closer, it was found that ˜25% (n=31) genes are interferon inducible genes or genes involved in the interferon/cytokine/JAK/STAT signaling pathways, Table 3. The suggested that the impairment of interferon signaling pathway might be important in early development of cancer (through an immortalization-related mechanism) and/or can be involved in the process of aging. The statistical probability of this happening by chance to ˜10⁻³⁴ was calculated.

TABLE 1
Affymetrix Microarray Data: genes regulated by immortalization
and methylation

| Accession # | Gene Name | IMMORT | 5aza | Software |
|---|---|---|---|---|
| L19686 | Microphage migration inhibitory | −278.0 | 42.1 | A/GS |
| | factor (MIF) | | | |
| X54489 | Melanoma growth stimulatory activity | −146.3 | 64.6 | A |
| | (MGSA) (GRO-1) | | | |
| M33882 | Interferon-induced p78, Mx1 | −99.3 | 202 | A/GS |
| AI017574 | Cysteine-rich heart protein | −85.0 | 8.8 | A |
| U66711 | Ly-6-related protein (9804) gene | −73.7 | 34.1 | A/GS |
| | (responsive to IFNs) | | | |
| X82494 | Fibulin-2 | −70.1 | 21.8 | A/GS |
| AF054825 | VAMP45 (vesicle-associated | −69.1 | 9.1 | A |
| | membrane protein 5) | | | |
| AL049946 | Adlican | −60.2 | 17.6 | A/GS |
| M33882 | Interferon-induced p78, MxB | −52.1 | 122.1 | A/GS* |
| M55153 | Transglutaminase (TGase) | −50.9 | 144.6 | A |
| AF037335 | Carbonic anhydrase precursor (CA | −47.4 | 8.9 | A |
| | 12) | | | |
| L24564 | Rad (Ras associated with diabetes) | −35.9 | 19.7 | A/GS* |
| AA631972 | Nk4 protein (natural killer cell | −35.0 | 20.2 | A/GS |
| | transcript 4) | | | |
| U20982 | Insulin-like growth factor binding | −33.3 | 3.8 | A* |
| | protein-4 | | | |
| AF039103 | Tat-interacting protein TIP30 | −30.3 | 8.9 | A |
| J09309 | Gamma-interferon-inducible protein | −27.8 | 31.2 | A/GS |
| | (IP-30) | | | |
| AF053944 | Aortic carboxypeptidase-like protein | −27.4 | 12.3 | A/GS |
| AL080059 | CDNA DKFZp564H142 | −23.4 | 9.5 | A |
| U88964 | HEM45 (interferon-stimulated gene, | −21.7 | 50.2 | A/GS |
| | 20-kd; ISG20) | | | |
| U03688 | Dioxin-inducible cytochrome P450 | −20.6 | 6.9 | A |
| | (CYP1B1) | | | |
| U59185 | Putative monocarboxylate | −19.5 | 6.3 | A |
| | transporter | | | |
| AB029000 | KIAA 1077 protein Sulfatase FP | −18.6 | 10.9 | A |
| U45878 | Inhibitor of apoptosis protein 1 | −18.4 | 13.1 | A/GS |
| M28130 | Interleukin 8 | −15.5 | 92.7 | A/GS |
| X04371 | 2-5A synthetase induced by | −15.4 | 76.5 | A/GS* |
| | interferon OAS-1 | | | |
| X02419 | uPA gene (urokinase-plasminogen | −14.6 | 4.4 | A |
| | activator gene) | | | |
| M13509 | Skin collagenase MMP1 | −14.4 | 4.8 | A |
| AF026941 | CIG5 (cytomegalovirus induces | −13.9 | 66.6 | A |
| | interferon-responsive) | | | |
| AB025254 | PCTAIRE 2 (pctaire protein kinase) | −13.7 | 19.3 | A/GS |
| X67325 | Interferon-stimulated gene p27 | −13.0 | 482.0 | A |
| | mRNA | | | |
| M36820 | Cytokine (GRO-beta, GRO-2) | −12.9 | 29.6 | A/GS |
| AF026939 | CIG49 (cytomegalovirus induces | −12.8 | 70.2 | A/GS |
| | interferon-responsive) | | | |
| M90657 | Tumor antigen (L6) | −12.6 | 17.5 | A/GS* |
| AF060228 | Retinoic acid receptor responder 3 | −12.2 | 7.1 | A/GS |
| J04164 | Interferon-inducible protein 9-27 | −11.8 | 8.8 | A |
| AI885852 | Similar to gb: L 19779 HISTONE | −7.3 | 10.3 | A/GS* |
| | H2A.1 | | | |
| M36821 | Cytokine (GRO-gamma) | −11.1 | 56.8 | A |
| AL050162 | TESTIN 3 testis derived transcript (3 | −10.7 | 8.1 | A |
| | LIM domains) | | | |
| U77643 | K12 protein precursor (SECTM1) | −10.7 | 19.6 | A/GS |
| D28137 | BST-2 (bone marrow stroma cell | −9.9 | 38.7 | A/GS |
| | surface gene) | | | |
| M17017 | Beta-thromboglobin-like protein | −9.7 | 17.5 | A/GS |
| AC004142 | BAC clone RG118D07 from 7q31 | −9.4 | 5.5 | A |
| M24283 | Major group rhinovirus receptor | −9.3 | 28.9 | A |
| | (HRV) | | | |
| AL022723 | HLA-F, gene for major | −8.9 | 29.4 | A/GS* |
| | histocompatibility complex class I F | | | |
| U15932 | Dual-specificity protein phosphatase | −8.9 | 12.6 | A/GS |
| M24594 | Interferon-inducible 56 Kd protein | −8.6 | 36.6 | A/GS* |
| AB020315 | Dickkopf-1 (hdkk-1) | −8.3 | 14.4 | GS |
| J02931 | Placental tissue factor (two forms) | −8.0 | 8.7 | A |
| X86163 | B2-bradykinin receptor, 3 | −7.9 | 3.8 | A |
| M13755 | Interferon-induced 17-kDa/15-kDa | −7.6 | 17.0 | A/GS* |
| | protein | | | |
| L20817 | Tyrosine protein kinase (CAK) gene | −7.5 | 5.7 | A |
| AJ225089 | 2-5 oligoadenylate synthetase 59 kDa | −7.4 | 40.0 | A/GS |
| | OAS-L | | | |
| AF085692 | Multidrug resistance-associated | −7.3 | 13.9 | A |
| | protein 3B | | | |
| M26326 | Keratin 18 | −6.9 | 13.5 | A |
| M22489 | Bone morphogenetic protein 2A | −6.8 | 9.9 | A |
| U37518 | TNF-related apoptosis inducing | −6.7 | 42.2 | A |
| | ligand TRAIL | | | |
| M92357 | B94 protein (tumor necrosis factor- | −6.7 | 5.5 | A |
| | alpha-inducible) | | | |
| X07523 | Complement factor H | −6.6 | 6.8 | A |
| AB018287 | KIAA0744 protein | −6.5 | 8.6 | A |
| U53831 | Interferon regulatory factor 7B | −6.3 | 17.5 | A/GS |
| X55110 | Neurite outgrowth-promoting protein | −6.2 | 7.1 | A |
| AL039458 | Integral membrane glycoprotein LIG- | −6.1 | 5.2 | A |
| | 1 (TM4SF1) | | | |
| AL021977 | Transcription Factor MAFF | −6.1 | 8.1 | GS |
| M65292 | Factor H homologue | −5.8 | 7.5 | A |
| D29992 | Placental protein 5 (PP5) | −5.7 | 29.3 | A |
| AF070533 | Optineurin-like protein | −5.7 | 4.2 | A** |
| AF052135 | Associated molecule with the SH3 | −5.6 | 7.6 | GS |
| | domain of STAM | | | |
| D28915 | Microtubular protein 44 | −5.5 | 17.8 | A/GS |
| M62402 | Insulin-like growth factor binding | −5.5 | 5.9 | A/GS |
| | protein 6 | | | |
| AF024714 | Interferon-inducible protein AIM2 | −5.3 | 21.6 | A |
| | (absent in melanoma) | | | |
| M31165 | Tumor necrosis factor-inducible | −5.2 | 10.3 | A |
| | (TSG-6) | | | |
| U81607 | Gravin | −5.1 | 12.2 | GS |
| M30818 | Interferon-inducible protein, | −5.0 | 42 | A |
| | myxovirus resistance, Mx2 | | | |
| M25915 | Complement cytolysis inhibitor (CLI) | −4.9 | 5.1 | A |
| AB013382 | DUSP6 (dual specificity MAP kinase- | −4.7 | 4.6 | A |
| | phosphatase) | | | |
| AB000115 | mRNA expressed in osteoblast | −4.3 | 32.5 | A/GS |
| D50919 | Tripartite motif-containing protein 14, | −4.3 | 6.2 | A |
| | TRIM14 | | | |
| X58536 | HLA class I locus C heavy chain | −4.1 | 5.7 | A |
| AF010312 | Pig 7 | −4 | 9.4 | GS |
| AI985272 | Neuromedin B Precursor | −3.9 | 5.6 | A |
| X57985 | Genes for histones H2B.1 and H2A | −3.6 | 4.3 | A |
| U07919 | Aldehyde dehydrogenase 6 | −3.5 | 4.1 | A |
| AA883502 | Ubiquitin-conjugating enzyme E2L6 | −3.4 | 5.9 | A |
| | (UBE2L6) | | | |
| U22970 | Interferon-inducible peptide (6-16) | −3.3 | 10.2 | GS |
| M87434 | 2-5 oligoadenylate synthetase 69/71 kDa | −2.7 | 19.6 | A |
| | OAS-2 | | | |
| M97935 | Transcription factor ISGF-3 (Stat 1) | −1.9 | 7.6 | A** |

Confirmation of Changes in Gene Expression

Immortal (PO 212) and pre-immortal (PO 11) fibroblasts cells (MOAH041 cell line) were used to analyze the changes in gene expression during immortalization. Total RNA was isolated from these cells and used as a probe for hybridization on microarrays. Affymetrix HGU95Av2 GeneChips were used and the data were analyzed using Affymetrix Microarray Suite and Data Mining Tool software packages (Affymetrix). The microarray data were further confirmed using Quantitative Real Time-PCR (Q-RT-PCR) using a randomly selected set of these genes. Table 2 shows a comparison of the levels of gene expression during immortalization by using both microarray hybridization and Q-RT-PCR. In all cases, 16 down-regulated and 5 up-regulated genes chosen by bioinformatics methods, there is an excellent correlation of the data obtained using both techniques. Since Q-RT-PCR data is accurate over a larger range of expression levels, the data obtained using microarrays and Q-RT-PCR are quantitatively different.

TABLE 2
Comparison of expression levels of genes differentially regulated
during immortalization* by Affymetrix microarray technology
and quantitative real-time PCR.

| | | |
|---|---|---|
| | | Q-RT-PCR, |
| Gene | Microarray, fold change | fold change |

Down-regulated genes

| | | |
|---|---|---|
| MIF | 277 | 65 |
| MGSA | 145 | 6700 |
| Interferon-inducible | 99.3 | 2700 |
| protein p78 | | |
| NDN | 60 | 2790 |
| CD24 | 45 | 7450 |
| CYP1B1 | 20 | 45 |
| (2-5′) oligoadenylate | 19 | 160 |
| synthetase E gene OAS1 | | |
| CIG49 | 13 | 36 |
| Interferon-inducible 56 kDA | 8.6 | 108 |
| Interferon-inducible | 12 | 8 |
| membrane protein 9-27 | | |
| (IFITM1) | | |
| Dermatopontin | 10 | 13 |
| Interferon-regulatory | 6 | 2683 |
| factor 7B | | |
| MRP3 | 5 | 17 |
| Interferon-induced 17-kDA/ | 4.5 | 43 |
| 15-kDA | | |
| GST4A | 4 | 8 |
| Signal Transducer and | 1.9 | 8.5 |
| Activator of Transcription | | |
| 1, STAT1, 91 kD | | |
| AIM2 | 5.3 | 165 |
| IP-30 | 27.8 | 12 |
| P69/OAS-2 | 2.7 | 142 |
| Interferon, alpha-inducible | 13 | 70 |
| p27 | | |

Up-regulated genes

| | | |
|---|---|---|
| WISP | 8 | 20 |
| SNF2A | 6 | 3 |
| ERCC2 | 5 | 4 |
| RAGE3 | 7 | 10 |
| HTERT | 1.6 | 486 |

*Fold change of gene expression level in the immortal cells (MDAHO41 high passage) relative to non-immortal cells (MDAHO41 low passage). Analysis of the genes involved in immortalization indicated that a large fraction of them are interferon (IFN) regulated, Table 3. Analysis of the chromosomal location of these IFN-regulated genes revealed that they are clustered in multiple loci around the human genome.

TABLE 3
Affymetrix Microarray Data: CYTOKINE/JAK/STAT pathway genes
regulated by demethylation and immortalization

| Gene | IMMORT | 5AZA | CpG | Locus |
|---|---|---|---|---|
| 1. Interferon-induced 17-kDa/15-kDA | −4.2 | 13.9 | + | 1p36.33 |
| 2. Interferon-inducible peptide (6-16) | −3.3 | 10.2 | − | 1p36 |
| 3. mRNA expressed in osteoblast | −4.3 | 32.5 | − | 1p31 |
| 4. Microtubular protein p44 (IFI44) | −5.5 | 17.8 | − | 1p31.1 |
| 5. Interferon-inducible protein | −5.3 | 21.6 | − | 1q22 |
| (Absent in Melanoma 2, AIM2) | | | | |
| 6. Complement factor H | −6.6 | 6.8 | − | 1q32 |
| 7. CIG5 vipirin; similar to Inflammatory | −13.9 | 67.0 | − | 2p25.3 |
| Response Protein 6 | | | | |
| 8. Signal Transducer Activator of | −1.9 | 7.6 | + | 2q32.2 |
| Transcription 1 STAT1 91 kDa | | | | |
| 9. TNF-related apoptosis inducing | −6.7 | 42.2 | − | 3q26 |
| ligand, TRAIL | | | | |
| 10. Cytokine (GRO-beta, GRO-2) | −12.9 | 29.6 | + | 4g12-13 |
| 11. Interleukin 8 | −15.5 | 92.7 | − | 4q12-13 |
| 12. HLA class I locus C heavy chain | −4.1 | 5.7 | + | 6p21 |
| 13. uPA gene (urokinase- | −14.6 | 4.4 | + | 8p12 |
| plasminogen activator gene) | | | | |
| 14. Ly-6-related protein (9804) gene | −73.7 | 34.1 | + | 8q24.3 |
| 15. Tripartite motif-containing protein | −4.3 | 6.2 | + | 9q22-q31 |
| 14, TRIM14 | | | | |
| 16. CIG49 Interferon-induced protein | −12.8 | 70.2 | − | 10q24 |
| with tetratricopepide repeats 4 | | | | |
| 17. Interferon-inducible 56 kDa | −8.6 | 36.6 | − | 10q25-q26 |
| protein | | | | |
| 18. Interferon-inducible membrane | −11.8 | 8.8 | − | 11p15.5 |
| protein 9-27 (IFITM1) | | | | |
| 19. Interferon regulatory factor 7B | −6.3 | 17.5 | + | 11p15.5 |
| 20. (2-5′) oligoadenylate synthetase | −15.4 | 76.5 | − | 12q24.1 |
| p46/p42 E gene OAS-1 | | | | |
| 21. (2-5′) oligoadenylate synthetase | −7.4 | 40.0 | − | 12q24.2 |
| 59 kDa isoform OAS-L | | | | |
| 22. (2-5′) oligoadenylate synthetase | −2.7 | 19.6 | − | 12q24.2 |
| 69/71 kDa isoform OAS-2 | | | | |
| 23. Interferon, alpha-inducible | −13.0 | 482.0 | − | 14q32 |
| protein 27 | | | | |
| 24. HEM45, ISG-20 | −21.7 | 50.2 | − | 15q26 |
| 25. NK4 protein (natural killer cell | −35.0 | 20.2 | − | 16p13.3 |
| transcript 4) | | | | |
| 26. Insulin-like growth factor binding | −33.3 | 3.8 | − | 17q12-q21 |
| protein-4 | | | | |
| 27. Gamma-interferon-inducible | −27.8 | 31.2 | + | 19p13.1 |
| protein (IP-30) | | | | |
| 28. BST-2 (bone marrow stroma cell | −9.9 | 38.7 | − | 19p13.2 |
| surface gene) | | | | |
| 29. Major group rhinovirus receptor (HRV) | −9.3 | 28.9 | + | 19p13.3 |
| ICAM | | | | |
| 30. Interferon-inducible protein p78, Mx2 | −99.3 | 202 | + | 21q22.3 |
| 31. Interferon-inducible protein Mx1 | −5 | 42 | − | 21q22.3 |

(Data was processed in Affymetrix Data Mining Tool. Triplicates were averaged.)
5aza: Up-regulation in 5-aza-CdR treated HP MDAHO41 cells vs. untreated HP MDAHO41 cells
041HP: down-regulation in HP MDAHO41 cells VS. LP MDAHO41 cells

Interferons

Interferons are a group of pleiotropic cytokines. Human interferons can be divided into two major classes, type-I (IFN alpha, beta, omega) and type-II (IFN gamma). Although they have common antiviral, antiproliferative and immunomodulatory activities (Platanias (1995); Platanias (1999), their physical and immunochemical properties are different (Platanias (1995). Interferons are generally inducible proteins, type-I IFNs are expressed in a various type of cells induced by viral infection. Type-II IFN is produced by activated T lymphocytes and natural killer cells. The diverse biological functions of interferons are realized by the expression of interferon inducible genes after the cells receive the signals from interferons. Type-I IFN receptor (IFNR) and type-II IFN receptor (IFNGR) are different and both type-I IFN and type-II IFN can induce several signaling pathways (Imada et al (2000). Jak-Stat pathway is one major pathway, which can be induced in both type-I and type-II IFNs. Upon the binding of interferon with its receptor, Jaks, receptor associated tyrosine kinase, are activated. Stats can then be recruited to the receptors via their SH2 domain and tyrosine phosphorylated by Jaks. Activated Stats can form homodimers or heterodimers, and then translocate to the nucleus to activate the expression of target genes that have proper promoter regulatory elements (Leonard et al (1998); Uddin et al (1996). Pathways involved in type-I interferon signaling also include insulin receptor substrate (IRS)/PI-3′-kinase pathway and pathways involving adaptor proteins of the Crk-family (CrkL and Crkll) or vav proto-oncogene product. For type II interferon stimulated pathways, besides Jaks, some other tyrosine kinases, Fyn (src-family) and Pyk-2 can also be activated. (Takaoka et al (1999); Pitha (2000). IFNs have shown their antiviral effects on several virally induced carcinomas and their influence in cell metabolism, growth and differentiation has suggested their importance in inhibiting tumorigenesis. A number of IFNs induced genes have tumor suppression activities when over expressed in uninfected cells, e.g. double stranded RNA activated protein kinase (PKR), activated RNAseL, and the proteins of the 200 gene family (Karpf et al (1999). Some recent studies in examining the promoter methylation in bladder cancer cells and colon adenocarcinoma cells also showed the activation of IFN signaling pathways after the treatment of 5-aza-CdR to cancer cells. The suggested IFN signaling pathway was found to be a potential tumor-suppressive pathway (Peris et al (1999; Agrawal et al (2002). The experimental results first revealed that IFN signaling pathways can be disrupted in immortalization. Based on the current knowledge of IFN signaling pathway and the present data, the promoter hypermethylation regulation of IFN signaling pathways appears to play a significant role in immortalization and identification of immortalization genes in IFN signaling pathways.

STAT 1

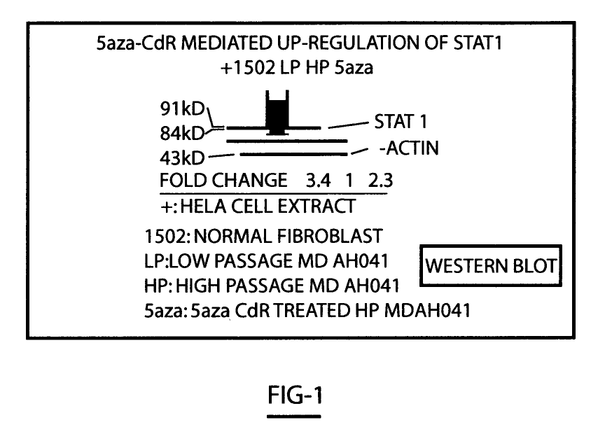

Signal transducers and activators of transcription 1 (STAT1) is one of the seven identified Stat proteins play an important role in cytokine signaling transduction. STAT1 is involved in both type-I and type-IIIFN signaling pathways. (FIGS. 1, 3) It forms homodimer or heterodimer with other Stat proteins to activates the genes who have IFN-stimulated response elements (ISRE) or IFN-gamma activated sequences (GAS). Although STAT1α can be induced by several kinds of cytokines and is involved in diverse signaling pathways, the predominant role for STAT1α a is suggested to be growth inhibition (Uddin et al (1996). The antiproliferative function of STAT1α is revealed by its induction of the CDK inhibitor p21^{WAF1} (Chin et al (1997), caspase 1 (Xu et al (1998), Fas and FasL (Kaplan et al (1998), which leads to cell cycle arrest and apoptosis. The deficiency of STAT1α can thus confer a selective advantage to tumor cells. In the study of STAT1α knockout mice, mice lacking STAT1α develops spontaneous and chemically induced tumors more rapidly and with more rapid frequency comparing with their wild-type littermates (Huang et al (2000). The regulation of STAT1α by promoter hypermethylation in tumor cells has been implicated in the study of colon cancer and bladder cancer cells (Peris et al (1999; Agrawal et al (2002). The negative regulatory effects of STAT1α in angiogenesis, tumorigenesis and metastasis have also been demonstrated in a transfection study in mouse fibrosarcoma (Altman et al (2001). These data combined with the findings suggest STAT1α to be a tumor suppressor gene involved in immortalization with the implication that IFN pathway genes are regulated by promoter hypermethylation. At a functional level, STAT1α could be a promising transcriptional regulator immortalization and cancer. The regulation of STAT1α at the mRNA level was confirmed by quantitative RT-PCR (Table 4 and at the protein level, FIG. 3). The genes regulated by demethylation were also tested by quantitative RT-PCR and their up regulation was confirmed, Table 4.

TABLE 4
Confirmation of expression levels of genes identified by Affymetrix
microarray technology as differentially upregulated during Saza-CdR
induced DNA demethylation* using Quantitative Real-Time PCR.

| | | |
|---|---|---|
| | Microarray, fold | Q-RT-PCR, |
| Gene | change | fold change |

Up-regulated genes

| | | |
|---|---|---|
| Interferon-inducible p78 | 202 | 478 |
| (2-5′) OAS1 | 92 | 4379 |
| CIG49 | 70 | 204 |
| MGAS | 65 | 839 |
| MIF | 42 | 128 |
| Interferon-inducible 56 kDa | 36.6 | 1807 |
| Interferon regulatory factor | 17.5 | 20031 |
| 7B | | |
| CYP1B1 | 7 | 77 |
| MRP3 | 14 | 54 |
| Interferon-induced 17/15-kDA | 14 | 228 |
| Interferon-inducible | 9 | 278 |
| membrane protein 9-27 | | |
| (IFITM1) | | |
| IP-30 | 31.2 | 7 |
| Signal Transducer and | 7.6 | 158 |
| Activator of Transcription 1, | | |
| STAT1, 91 kD | | |
| Interferon, alpha-inducible | 482 | 1320 |
| p27 | | |
| P69/OAS-2 | 19.6 | 231 |
| AIM2 | 21.6 | 686 |

*Fold change of gene expression in the immortal cells treated with 5aza-CdR relative to untreated cells.

TABLE 5
Comparison of expression level of genes differentially
regulated in immortal** and normal* cells after
5azaCdR-induced DNA demethylation.

| | | MDAH041HP vs. |
|---|---|---|
| | 1502 vs. | MDAH0411HP |
| Gene Name | 1502 5aza* | 5aza** |
| STAT 1, 91 kD | 1.8↑ | 158↑ |
| Interferon-inducible protein p78 | 1.8↑ | 480↑ |
| MIF | 1.7↓ | 130↑ |
| MGSF | 3.2↓ | 800↑ |
| NDN | 1.4↓ | 10↑ |
| Interferon-inducible 56 kDa | 1.2↓ | 1807↑ |
| protein | | |
| Interferon-inducible membrane | 1.8↓ | 278↑ |
| protein 9-27 (IFITM1) | | |
| Interferon-induced 17-kDa/15-kDA | 1.8↑ | 443↑ |
| (2-5′) oligoadenylate synthetase | 2↑ | 1072↑ |
| E gene OAS1 | | |
| CIG49 | 1.1↑ | 204↑ |
| Interferon-regulatory factor 7B | 1.5↑ | 20031↑ |

*Fold change of gene expression level in a non-immortal normal skin fibroblast (NSF) cell line 1502 before and after treatment with 5azaCdR.
**Fold change of gene expression level in an immortal fibroblast cell line (041, high passage) before and after treatment with 5azaCdR.
# ↑ and ↓ indicate increase and decrease in gene expression, respectively.

To determine the specificity and significance of these findings, the expression levels of 11 genes in normal fibroblast cells (strain 1502) with 5-aza-CdR treated or untreated using Q-RT-PCR Table 5 were analyzed. Treatment of nonimmortal cells with 5Aza-dC does not result in an induction of an senescence-like state in the cells. When the expression levels of 11 of these genes were analyzed in normal fibroblast cells (strain 1502) 5Aza-dC treated or untreated using Q-RT-PCR (Table 5) no 5Aza-dC dependent changes in expression were observed. None of these genes were significantly altered in their expression after the 5-aza-CdR-treatment.

In summary. while 5Aza-dC-treatment strongly induces expression of many genes in Immortal cells. expression of the same genes is not significantly altered after the 5Aza-dC-treatment of normal fibroblasts. Therefore the immortal-specific gene expression changes observed in immortal MDAHO41 cells also regulated by treatment with 5Aza-dC has identified gene targets of cellular immortalization that were silenced by methylation.

### Example 3

The genes listed in Table 8 were increased (decreased) across four independently immortalized cell lines: MDAH041, MDAH087-N, MDAH087-1 and MDAH087-10. All three variants are derived from an original cell line. Each variant has different germlne p53 mutations, however all lose their wild type p53 upon immortalization. If a gene increased (decreased) across less then 4/4 of the cell lines, the gene is not present in these lists.

Several situations could exist: 1. Genes decreased after treatment with 5-aza-deoxycidine (5-aza-dC); 2. Genes increased after treatment with 5-aza-dC; 3. Genes decreased during immortalization; 4. Genes increased during immortalization; and 5. Intersection of the genes that decreased during immortalization and increased after treatment with 5-aza-dC (Intersection of lists 1 and 4). For 1 and 2, 5-aza-dC treated immortalized cells were compared to untreated immortalized cells. For 3 and 4, immortalized cells were compared to pre-crisis cells. MDAH041 immortal cells were compared to MDAH041 pre-crisis cells. MDAH087-N, MDAH087-1 and MDAH087-10 were compared to MDAH087 pre-crisis cells.

The Affymetrix probe ID for a probe. A probe is a sequence that is unique to 1 gene. Note, there are sometimes multiple probes for 1 gene. The microarry chip used was HG-U95Av2.

There were multiple microarray chips, representing independent experiments, for each cell line. First we determined the genes that increased (decreased), during immortalization, across all chip comparisons for an individual cell line. Similarly, we determined the genes that increased (decreased), after treatment with 5-aza-dC, across all chip comparisons for an individual cell line. We used the list of genes generated for the individual cell lines to determine genes that were in common across all four cell lines. The results are shown in Table 8.

p53 sequence analysis of
LFS patients' fibroblasts

| | | |
|---|---|---|
| Cell Line | Codon | Mutation Type |

| | | |
|---|---|---|
| MDAH087 | 248 CGG/TGG | Arg to Trp |
| MDAH172 | 175 CGC/CAC | Arg to His |
| MDAH174 | 175 CGC/CAC | Arg to His |
| MAT170-1 | 133 ATG/ACG | Met to Thr |
| MAT170-3 | 133 ATG/ACG | Met to Thr |
| MAT120-1 | N.D. | wt by Western Blot |
| MDAH041 | 184 GAT A/GAA | Frameshift stop after |
| | | 60 amino acids |

ND = not determined

| | NUMBER OF GENES | | | | | | |
|---|---|---|---|---|---|---|---|
| | | | | | MDAH087-N | | |
| | | | | | MDAH087-1 | Magic Bullets | |
| | MDAH041 | MDAH087-N | MDAH087-1 | MDAH087-10 | MDAH087-10 | Total | |
| | Total Probe | Total Probe | Total Probe | Total Probe | Total Probe | Probe | Unique |
| Gene Set | IDs | IDs | IDs | IDs | IDs | IDs | Unignes |
| A. IM vs PC | 440 | 576 | 796 | 332 | 136 | 26 | 26 |
| upregulated* | | | | | | | |
| B. IM vs PC | 625 | 486 | 613 | 467 | 221 | 85 | 80 |
| downregulated* | | | | | | | |
| C. IM 5azaCdR | 420 | 311 | 266 | 134 | 40 | 6 | 6 |
| vs IM untreated, | | | | | | | |
| downregulated** | | | | | | | |
| D. IM 5azaCdR | 547 | 447 | 329 | 306 | 125 | 85 | 76 |
| vs IM untreated | | | | | | | |
| upregulated** | | | | | | | |
| Genes in Sets | 119 | 52 | 44 | 33 | 8 | 4 | 3 |
| B and D# | | | | | | | |
| Genes in Sets | 30 | 101 | 72 | 24 | 3 | 0 | 0 |
| A and C# | | | | | | | |

| | | | | | | |
|---|---|---|---|---|---|---|
| P: Present | Downregulated | Downregulated | Downregulated | Downregulated | Downregulated | Downregulated |
| M: Marginal | Genes: | Genes: | Genes: | Genes: | Genes: | Genes: |
| A: Absent | Call: P M A | Call: P M A | Call: P M A | Call: P M A | Call: P M A | Call: P M A |
| I: Increase | Change: D MD | Change: D MD | Change: D MD | Change: D MD | Change: D MD | Change: D MD |
| MI: Marginal | Upregulated Genes: | Upregulated | Upregulated | Upregulated | Upregulated | Upregulated |
| Increase | Call: P M | Genes: | Genes: | Genes: | Genes: | Genes: |
| D: Decrease | Change: I MI | Call: P M | Call: P M | Call: P M | Call: P M | Call: P M |
| MD: Marginal | | Change: I MI | Change: I MI | Change: I MI | Change: I MI | Change: I MI |
| Decrease | | | | | | |

Fold change: No fold Percent comparisons: 100% Total number of comparisons in ( )
Notes:
¹Low passage/Pre-crisis chips used: PC2, PC6
High passage/Immortalized chips used: AE1, AE2, OE2
5-aza-dC treated immortal chips used: AE1, AE2, AE4

| (B-D)Intersection_magic_bullets | | | | | | | | | | |
|---|---|---|---|---|---|---|---|---|---|---|
| | | | | | PC vs HP | | 041 5-aza | | PC vs IM | |
| | | | | | 041 HP Ave | | Ave Signal | | N-UT Ave | |
| Probe ID | Unigene | Locus ID | Symbol | Chromosome | Signal Log | | Log | | Signal Log | |
| 36686_at | Hs.75748 | 220 | ALDH1A3 | 15q26.3 | −1.42 | −2.67 | 1.50 | 2.83 | −1.18 | −2.26 |
| 40071_at | Hs.154654 | 1545 | CYP1B1 | 2p21 | −3.47 | −11.04 | 1.91 | 3.75 | −3.03 | −8.16 |
| 859_at | Hs.154654 | 1545 | CYP1B1 | 2p21 | −3.81 | −14.04 | 3.03 | 8.17 | −3.06 | −8.36 |
| 32730_at | Hs.173094 | 85453 | KIAA1750 | 8q22.1 | −4.80 | −27.76 | 1.53 | 2.88 | −4.23 | −18.61 |
| | | | | | Log(2) | | | | | |

| (B-D)Intersection_magic_bullets | | | | | | | | | | |
|---|---|---|---|---|---|---|---|---|---|---|
| | | | | | | | | | 10-5aza | |
| | N-5aza Ave | | 1-UT Ave | | 1-5aza Ave | | 10-UT Ave | | Ave Signal | |
| Probe ID | Signal Log | | Signal Log | | Signal Log | | Signal Log | | Log | |
| 36686_at | 1.70 | 3.24 | −3.00 | −8.00 | 1.19 | 2.29 | −2.15 | −4.42 | 1.84 | 3.83 |
| 40071_at | 2.95 | 7.73 | −1.85 | −3.60 | 2.06 | 4.16 | −2.39 | −5.25 | 2.23 | 4.70 |
| 659_at | 3.00 | 7.98 | −2.06 | −4.17 | 2.29 | 4.69 | −2.36 | −5.13 | 2.23 | 4.69 |
| 32730_at | 2.35 | 5.10 | −1.90 | −3.72 | 1.71 | 3.28 | −3.93 | −15.24 | 3.18 | 9.09 |

| (A) HP_I_Annotated_Averages | | | | | | | | | | |
|---|---|---|---|---|---|---|---|---|---|---|
| | | | | | | | 041 5-aza | | | |
| | | | | | 041 HP Ave | | Ave Fold | | N-UT Ave | |
| Probe ID | Unigene | Locus ID | Symbol | Chromosome | Fold Change | | Change | | Fold Change | |
| 32331_at | Hs.274691 | 205 | AK3 | 1p31.3 | 1.34 | 2.52 | −0.02 | −1.02 | 2.03 | 3.11 |
| 39230_at | Hs.226307 | 9582 | APOBEC38 | 22q13.1-q13.2 | 0.92 | 1.89 | −1.27 | −2.40 | 1.46 | 2.86 |
| 38201_at | Hs.317432 | 586 | BCAT1 | 12pter-q12 | 3.08 | 8.47 | 0.66 | 1.58 | 1.51 | 4.52 |
| 32238_at | Hs.193163 | 274 | BIN1 | 2q14 | 1.72 | 3.29 | 0.64 | 1.56 | 0.66 | 3.23 |
| 35615_at | Hs.30736 | 23246 | BOP1 | 6q24.3 | 1.47 | 2.77 | 0.23 | 1.18 | 1.07 | 2.08 |
| 1942_s_at | Hs.95577 | 1019 | CDK4 | 12q14 | 0.53 | 1.44 | −0.49 | −1.41 | 0.96 | 2.26 |
| 37931_at | Hs.85004 | 1059 | CENPB | 20p13 | 0.96 | 1.95 | 0.16 | 1.12 | 0.99 | 2.23 |
| 39231_at | Hs.22670 | 1105 | CHD1 | 5q15-q21 | 0.87 | 1.83 | −0.03 | −1.02 | 1.10 | 1.77 |
| 33650_at | Hs.65234 | 55681 | DDX27 | 20q13.13 | 1.18 | 2.26 | 0.21 | 1.15 | 0.68 | 2.21 |
| 1537_at | Hs.77432 | 1958 | EGFR | 7p12 | 2.23 | 4.69 | 1.10 | 2.14 | 3.59 | 2.76 |
| 40845_at | Hs.256583 | 3609 | ILF3 | 19p13.2 | 1.44 | 2.70 | 0.19 | 1.14 | 1.42 | 2.08 |
| 36624_at | Hs.75432 | 3615 | IMPDH2 | 3p21.2 | 0.81 | 1.53 | −0.59 | −1.50 | 0.71 | 4.72 |
| 39926_at | Hs.37501 | 4090 | MADH5 | 5q31 | 1.06 | 2.08 | 0.48 | 1.39 | 1.05 | 2.01 |
| 673_at | Hs.172665 | 4522 | MTHFD1 | 14q24 | 1.55 | 2.93 | −0.56 | −1.48 | 1.01 | 2.07 |
| 1973_s_at | Hs.79070 | 4609 | MYC | 8q24.12-q24.13 | 1.76 | 3.36 | 0.42 | 1.33 | 2.24 | 1.63 |
| 1979_s_at | Hs.15243 | 4839 | NOL1 | 12p13 | 1.03 | 2.04 | 0.85 | 1.81 | 1.06 | 2.88 |
| 35705_at | Hs.37288 | 9975 | NR1D2 | 3p24.1 | 1.44 | 2.71 | 1.17 | 2.24 | 1.46 | 12.06 |
| 36125_s_at | Hs.74111 | 22913 | RALY | 20q11.21-q11.23 | 0.93 | 1.90 | 0.17 | 1.12 | 1.14 | 1.84 |
| 39731_at | Hs.146381 | 27316 | RBMX | Xq26 | 0.54 | 1.45 | −0.94 | −1.92 | 0.82 | 2.14 |
| 41363_at | Hs.102456 | 8487 | SIP1 | 14q13 | 1.23 | 2.34 | 0.36 | 1.28 | 1.16 | 1.99 |
| 38455_at | Hs.83753 | 6628 | SNRP8 | 20p13 | 0.74 | 1.67 | −0.44 | −1.35 | 1.18 | 1.94 |
| 34851_at | Hs.250822 | 6790 | STK6 | 20q13.2-q13.3 | 0.54 | 1.46 | −1.53 | −2.88 | 1.06 | 2.10 |
| 318_at | Hs.75307 | 7052 | TGM2 | 20q12 | 1.46 | 2.74 | 0.20 | 1.15 | 1.69 | 1.58 |
| 910_at | Hs.105097 | 7083 | TK1 | 17q23.2-q25.3 | 1.37 | 2.58 | −0.96 | −1.94 | 2.18 | 2.84 |
| 1581_s_at | Hs.75248 | 7155 | TOP2B | 3p24 | 1.77 | 3.40 | 0.32 | 1.25 | 1.52 | 2.74 |
| 40792_s_at | Hs.367689 | 7204 | TRIO | 5p15.1-p14 | 1.37 | 2.58 | 1.04 | 2.05 | 1.64 | 4.08 |

| (A) HP_I_Annotated_Averages | | | | | | | | | | |
|---|---|---|---|---|---|---|---|---|---|---|
| | N-5aza Ave | | 1-UT Ave | | 1-5aza Ave | | 10-UT Ave | | 10-5aza Ave | |
| Probe ID | Fold Change | | Fold Change | | Fold Change | | Fold Change | | Fold Change | |
| 32331_at | −0.54 | −1.46 | 1.42 | 2.68 | −1.03 | −2.04 | 1.84 | 3.57 | −0.87 | −1.82 |
| 39230_at | −1.03 | −2.05 | 1.55 | 2.92 | 0.17 | 1.12 | 1.42 | 2.67 | 0.92 | 1.89 |
| 38201_at | 0.46 | 1.37 | 1.71 | 3.28 | 0.65 | 1.80 | 1.96 | 3.89 | 0.16 | 1.12 |
| 32238_at | 0.44 | 1.36 | 0.89 | 1.86 | −0.17 | −1.12 | 0.88 | 1.84 | 0.13 | 1.09 |
| 35615_at | 0.36 | 1.28 | 1.31 | 2.48 | −0.24 | −1.18 | 1.12 | 2.17 | −0.03 | −1.02 |
| 1942_s_at | −0.54 | −1.46 | 1.25 | 2.37 | −0.33 | −1.25 | 1.07 | 2.09 | −0.16 | −1.12 |
| 37931_at | −0.47 | −1.38 | 0.92 | 1.69 | −0.47 | −1.38 | 1.02 | 2.03 | −0.38 | −1.30 |
| 39231_at | −0.37 | −1.29 | 0.97 | 1.95 | 0.07 | 1.05 | 0.86 | 1.82 | 0.30 | 1.23 |
| 33650_at | 0.30 | 1.23 | 0.79 | 1.73 | 0.23 | 1.18 | 1.12 | 2.18 | −0.28 | −1.22 |
| 1537_at | −0.18 | −1.14 | 3.12 | 8.67 | 0.39 | 1.31 | 2.96 | 7.77 | 0.85 | 1.81 |
| 40845_at | −0.24 | −1.16 | 1.08 | 2.12 | −0.09 | −1.07 | 1.37 | 2.59 | 0.31 | 1.24 |
| 36624_at | −0.28 | −1.21 | 0.95 | 1.93 | −0.22 | −1.16 | 0.79 | 1.73 | −0.15 | −1. |
| 39926_at | 0.20 | 1.15 | 0.92 | 1.89 | 0.42 | 1.34 | 1.35 | 2.55 | 0.27 | 1 |
| 673_at | −0.03 | −1.02 | 1.93 | 3.81 | −0.43 | −1.35 | 1.07 | 2.10 | −0.06 | −1.04 |
| 1973_s_at | −0.25 | −1.19 | 1.34 | 2.52 | −0.15 | −1.11 | 1.20 | 2.30 | 0.15 | 1.11 |
| 1979_s_at | 1.02 | 2.02 | 1.90 | 3.73 | 0.37 | 1.29 | 1.27 | 2.41 | 0.80 | 1.74 |
| 35705_at | −0.17 | −1.13 | 1.33 | 2.51 | 0.02 | 1.02 | 1.47 | 2.77 | 0.05 | 1.04 |
| 36125_s_at | −0.02 | −1.01 | 0.82 | 1.77 | 0.18 | 1.13 | 1.04 | 2.05 | 0.36 | 1.28 |
| 39731_at | −0.55 | −1.46 | 1.01 | 2.01 | −0.62 | −1.54 | 0.77 | 1.70 | −0.39 | 1.31 |
| 41363_at | 0.50 | 1.41 | 1.64 | 3.11 | −0.06 | −1.05 | 0.87 | 1.83 | 0.51 | 1.42 |
| 38455_at | 0.07 | 1.05 | 1.03 | 2.01 | 0.28 | 1.21 | 1.03 | 2.04 | 0.52 | 1.43 |
| 34851_at | −1.12 | −2.18 | 1.13 | 2.18 | −0.04 | −1.03 | 1.22 | 2.33 | 0.02 | 1.01 |
| 318_at | −0.36 | −1.28 | 2.44 | 5.44 | −0.28 | −1.21 | 1.40 | 2.63 | 0.51 | 1.43 |
| 910_at | −1.30 | −2.48 | 1.73 | 3.31 | 0.11 | 1.08 | 1.87 | 3.19 | 0.28 | 1.22 |
| 1581_s_at | −0.21 | −1.16 | 1.09 | 2.13 | −0.42 | −1.33 | 1.31 | 2.48 | −0.22 | −1.17 |
| 40792_s_at | −0.05 | −1.03 | 1.48 | 2.79 | −0.12 | −1.09 | 1.46 | 2.75 | 0.29 | 1.23 |

| (B) HP_D_Annotated_Averages | | | | | | | | | |
|---|---|---|---|---|---|---|---|---|---|
| | | | | 041 HP Ave | | 041 5-aza | | N-UT Ave | |
| | | | | Fold | | Ave Fold | | Fold | |
| Probe ID | Unigene | Locus ID | Symbol | Change | | Change | | Change | |
| 32755_at | Hs.195851 | 59 | ACTA2 | −3.97 | −15.65 | 0.04 | 1.03 | −2.56 | −5.91 |
| 39063_at | Hs.118127 | 70 | ACTC | −3.08 | −8.43 | 0.10 | 1.07 | −6.50 | −90.30 |
| 36686_at | Hs.75746 | 220 | ALDH1A3 | −1.42 | −2.67 | 1.50 | 2.83 | −1.18 | −2.26 |
| 32527_at | Hs.74120 | 10974 | APM2 | −6.79 | −110.66 | 1.36 | 2.57 | −5.38 | −41.50 |
| 39043_at | Hs.433506 | 10095 | ARPC18 | −0.87 | −1.83 | 1.08 | 2.12 | −1.52 | −2.86 |
| 41776_at | Hs.279910 | 475 | ATOX1 | −0.72 | −1.64 | 0.32 | 1.25 | −0.99 | −1.98 |
| 36497_at | Hs.57548 | 113146 | C14orf78 | −0.96 | −1.94 | −0.60 | −1.52 | −2.56 | −5.90 |
| 37112_at | Hs.101359 | 9750 | C6orf32 | −5.23 | −37.57 | 2.80 | 6.97 | −2.57 | −5.93 |
| 41207_at | Hs.18075 | 23733 | C9orf3 | −1.43 | −2.69 | 1.92 | 3.78 | −2.60 | −6.05 |
| 38418_at | Hs.82932 | 595 | CCND1 | −1.84 | −3.57 | 1.14 | 2.20 | −1.04 | −2.06 |
| 2020_at | Hs.82932 | 595 | CCND1 | −1.90 | −3.73 | 1.19 | 2.27 | −1.01 | −2.01 |
| 39351_at | Hs.278573 | 966 | CD59 | −0.91 | −1.88 | −0.31 | −1.24 | −0.84 | −1.79 |
| 41138_at | Hs.433387 | 4267 | CD99 | −1.49 | −2.80 | 0.58 | 1.49 | −1.44 | −2.70 |
| 32363_at | Hs.194687 | 9023 | CH25H | −4.42 | −21.46 | 1.03 | 2.04 | −4.17 | −18.04 |
| 40698_at | Hs.85201 | 9976 | CLECSF2 | −3.97 | −15.71 | −0.44 | −1.36 | −2.64 | −6.23 |
| 34203_at | Hs.21223 | 1264 | CNN1 | −3.25 | −9.51 | 0.85 | 1.80 | −3.58 | −11.93 |
| 39031_at | Hs.421621 | 1346 | COX7A1 | −4.76 | −27.00 | 2.70 | 6.51 | −7.34 | −161.83 |
| 32242_at | Hs.408767 | 1410 | CRYAB | −3.89 | −14.79 | 0.69 | 1.61 | −3.468 | −10.99 |
| 32243_g_at | Hs.408767 | 1410 | CRYAB | −3.66 | −12.63 | −0.27 | −1.21 | −3.51 | −11.42 |
| 859_at | Hs.154654 | 1545 | CYP1B1 | −3.81 | −14.04 | 3.03 | 8.17 | −3.06 | −8.36 |
| 40071_at | Hs.154654 | 1545 | CYP1B1 | −3.47 | −11.04 | 1.91 | 3.75 | −3.03 | −8.16 |
| 39140_at | Hs.95665 | 54505 | DDXx | −0.72 | −1.65 | −0.26 | −1.20 | −1.03 | −2.04 |
| 33337_at | Hs.185973 | 8560 | DEGS | −0.53 | −1.44 | −0.38 | −1.30 | −1.35 | −2.55 |
| 37000_at | Hs.76285 | 25874 | DKFZP564B167 | −0.66 | −1.58 | −0.17 | −1.13 | −1.46 | −2.74 |
| 36861_at | Hs.72157 | 25878 | DKFZp56411922 | −3.65 | −12.54 | 2.50 | 5.68 | −4.03 | −16.30 |
| 35977_at | Hs.40499 | 22943 | DKK1 | −2.97 | −7.82 | 1.62 | 3.07 | −0.85 | −1.80 |
| 36133_at | Hs.349499 | 1832 | DSP | −2.44 | −5.42 | 0.03 | 1.02 | −6.45 | −87.53 |
| 37600_at | Hs.81071 | 1893 | ECM1 | −2.00 | −4.00 | 0.07 | 1.05 | −1.30 | −2.46 |
| 39098_at | Hs.9295 | 2006 | ELN | −2.45 | −5.46 | 1.12 | 2.17 | −2.75 | −6.71 |
| 39861_at | Hs.119257 | 2017 | EMS1 | −1.34 | −2.53 | −0.50 | −1.41 | −1.12 | −2.17 |
| 41385_at | Hs.103839 | 23136 | EPB41L3 | −7.02 | −129.64 | −0.90 | −1.87 | −4.64 | −24.85 |
| 32148_at | Hs.183738 | 10160 | FARP1 | −1.16 | −2.24 | −0.45 | −1.36 | −2.03 | −4.09 |
| 39038_at | Hs.11494 | 10516 | FBLN5 | −2.11 | −4.31 | −0.17 | −1.13 | −1.86 | −3.63 |
| 37743_at | Hs.79226 | 9638 | FEZ1 | −4.46 | −22.01 | 0.22 | 1.16 | −3.52 | −11.48 |
| 38651_at | Hs.103419 | 9637 | FEZ2 | −0.63 | −1.55 | −0.15 | −1.11 | −0.51 | −1.42 |
| 40468_at | Hs.301763 | 23048 | FNBP1 | −0.69 | −1.61 | −0.38 | −1.30 | −0.73 | −1.65 |
| 35785_at | Hs.336429 | 23710 | GABARAPL1 | −3.77 | −13.64 | 1.69 | 3.22 | −1.05 | −2.08 |
| 905_at | Hs.3764 | 2987 | GUK1 | −0.56 | −1.47 | 0.57 | 1.49 | −0.71 | −1.63 |
| 38824_at | Hs.90753 | 10553 | HTATIP2 | −4.15 | −17.69 | 2.49 | 5.61 | −2.73 | −6.61 |
| 39781_at | Hs.1516 | 3487 | IGFBP4 | −4.31 | −19.86 | 1.48 | 2.80 | −1.69 | −3.22 |
| 38636_at | Hs.102171 | 3671 | ISLR | −2.04 | −4.12 | 0.58 | 1.49 | −2.52 | −5.72 |
| 35318_at | Hs.5737 | 9917 | KIAA0475 | −0.72 | −1.65 | −1.00 | −2.00 | −0.91 | −1.87 |
| 36453_at | Hs.5333 | 9920 | KIAA0711 | −4.33 | −20.04 | 0.33 | 1.26 | −4.11 | −17.29 |
| 41585_at | Hs.49500 | 23231 | KIAA0746 | −3.82 | −14.09 | −1.65 | −3.13 | −2.66 | −6.31 |
| 32730_at | Hs.173094 | 85453 | KIAA1750 | −4.80 | −27.76 | 1.53 | 2.88 | −4.23 | −18.81 |
| 38972_at | Hs.109438 | 115207 | LOC115207 | −2.63 | −6.18 | 0.51 | 1.42 | −2.43 | −5.37 |
| 35917_at | Hs.194301 | 4130 | MAP1A | −1.49 | −2.82 | −0.68 | −1.60 | −1.71 | −3.28 |
| 34403_at | Hs.3745 | 4240 | MFGE8 | −4.02 | −16.26 | −0.18 | −1.14 | −1.55 | −2.92 |
| 33447_at | Hs.180224 | 10627 | MLCB | −1.23 | −2.34 | 1.28 | 2.42 | −2.71 | −6.55 |
| 36073_at | Hs.50130 | 4692 | NDN | −7.57 | −190.46 | −0.25 | −1.19 | −1.92 | −3.79 |
| 38750_at | Hs.8546 | 4854 | NOTCH3 | −2.62 | −6.13 | −0.19 | −1.14 | −2.99 | −7.92 |
| 41742_s_at | Hs.278898 | 10133 | OPTN | −1.57 | −2.98 | 1.38 | 2.59 | −0.86 | −1.82 |
| 32260_at | Hs.194673 | 8682 | PEA15 | −0.72 | −1.64 | −0.64 | −1.55 | −1.66 | −3.15 |
| 40434_at | Hs.18426 | 5420 | PODXL | −4.54 | −23.18 | 0.10 | 1.07 | −3.59 | −12.03 |
| 35841_at | Hs.441072 | 5441 | POLR2L | −0.65 | −1.57 | −0.70 | −1.62 | −1.25 | −2.38 |
| 503_at | Hs.441072 | 5441 | POLR2L | −0.71 | −1.64 | −0.64 | −1.56 | −1.13 | −2.19 |
| 34797_at | Hs.406043 | 8611 | PPAP2A | −1.41 | −2.65 | 0.16 | 1.12 | −1.47 | −2.77 |
| 39366_at | Hs.303090 | 5507 | PPP1R3C | −1.89 | −3.70 | −0.31 | −1.24 | −0.85 | −1.80 |
| 36533_at | Hs.302085 | 5740 | PTG1S | −4.35 | −20.35 | −0.07 | −1.05 | −4.01 | −16.09 |
| 39244_at | Hs.119007 | 5867 | RAB4A | −5.16 | −35.63 | 1.13 | 2.19 | −3.54 | −11.65 |
| 38264_at | Hs.90875 | 5877 | RABIF | −2.22 | −4.66 | 0.12 | 1.08 | −0.86 | −1.82 |
| 38331_at | Hs.96038 | 6016 | RIT1 | −1.84 | −3.58 | 0.64 | 1.55 | −0.74 | −1.67 |
| 32827_at | Hs.206097 | 22800 | RRAS2 | −0.77 | −1.70 | −0.49 | −1.41 | −1.16 | −2.23 |
| 39338_at | Hs.400250 | 6281 | S100A10 | −1.23 | −2.35 | −0.26 | −1.20 | −1.88 | −3.69 |
| 38138_at | Hs.417004 | 6282 | S100A11 | −0.60 | −1.52 | 0.45 | 1.36 | −1.70 | −3.26 |
| 38087_s_at | Hs.81256 | 6275 | S100A4 | −2.06 | −4.18 | −0.14 | −1.10 | −2.13 | −4.39 |
| 39775_at | Hs.151242 | 710 | SERPING1 | −2.46 | −5.51 | −0.06 | −1.04 | −1.53 | −2.89 |
| 34993_at | Hs.151899 | 6444 | SGCD | −2.52 | −5.74 | −1.10 | −2.14 | −2.29 | −4.88 |
| 39260_at | Hs.351306 | 9122 | SLC16A4 | −5.48 | −44.53 | 3.33 | 10.03 | −2.37 | −5.16 |
| 32574_at | Hs.77813 | 6609 | SMPD1 | −1.08 | −2.11 | 0.42 | 1.34 | −1.10 | −2.14 |
| 1686_g_at | Hs.296169 | 10638 | SPHAR | −5.03 | −32.56 | 0.07 | 1.05 | −3.64 | −12.47 |
| 40419_at | Hs.160483 | 2040 | STOM | −1.63 | −3.10 | 0.52 | 1.43 | −2.51 | −5.70 |
| 35832_at | Hs.70823 | 23213 | SULF1 | −4.97 | −31.31 | 2.02 | 4.05 | −8.08 | −270.60 |
| 36931_at | Hs.433399 | 6876 | TAGLN | −1.15 | −2.21 | 0.84 | 1.79 | −1.38 | −2.59 |
| 1596_g_at | Hs.89640 | 7010 | TEK | −2.97 | −7.82 | −0.58 | −1.49 | −5.02 | −32.37 |
| 37643_at | Hs.82359 | 355 | TNFRSF6 | −2.27 | −4.82 | 0.40 | 1.32 | −2.64 | −6.21 |
| 1441_s_at | Hs.82359 | 355 | TNFRSF6 | −4.46 | −22.06 | 2.09 | 4.25 | −2.96 | −7.80 |
| 32313_at | Hs.300772 | 7169 | TPM2 | −1.16 | −2.23 | −0.42 | −1.34 | −1.64 | −3.12 |
| 32314_g_at | Hs.300772 | 7169 | TPM2 | −0.72 | −1.65 | −0.01 | −1.01 | −1.30 | −2.47 |
| 39331_at | Hs.336780 | 7280 | TUB8 | −0.44 | −1.36 | −0.51 | −1.43 | −1.97 | −3.90 |
| 32533_s_at | Hs.74669 | 10791 | VAMP5 | −2.84 | −7.18 | 0.86 | 1.81 | −0.85 | −1.80 |
| 40147_at | Hs.157236 | 10493 | VAT1 | −0.68 | −1.60 | −0.29 | −1.22 | −0.48 | −1.39 |
| 36170_at | Hs.7486 | 23474 | YF13H12 | −1.10 | −2.14 | 0.53 | 1.44 | −1.30 | −2.46 |
| 39170_at | Hs.99768 | | | −1.10 | −2.15 | −0.55 | −1.46 | −1.07 | −2.10 |
| 39162_at | Hs.356224 | | | −0.70 | −1.62 | −1.23 | −2.35 | −0.77 | −1.71 |

| (B) HP_D_Annotated_Averages | | | | | | | | | | |
|---|---|---|---|---|---|---|---|---|---|---|
| | N-5aza Ave | | 1-UT Ave | | 1-5aza Ave | | 10-UT Ave | | 10-5aza | |
| | Fold | | Fold | | Fold | | Fold | | Ave Fold | |
| Probe ID | Change | | Change | | Change | | Change | | Change | |
| 32755_at | −0.73 | −1.66 | −2.04 | −4.11 | −1.55 | −2.92 | −1.34 | −2.53 | −1.71 | −3.28 |
| 39063_at | 0.43 | 1.35 | −7.45 | −174.85 | 1.22 | 2.33 | −7.32 | −159.79 | −0.29 | 1.22 |
| 36686_at | 1.70 | 3.24 | −3.00 | −8.00 | 1.19 | 2.29 | −2.15 | −4.42 | 1.94 | 3.83 |
| 32527_at | 0.04 | 1.03 | −5.17 | −35.92 | 2.93 | 7.60 | −2.72 | −6.60 | 0.25 | 1.19 |
| 39043_at | 0.10 | 1.07 | −0.99 | −1.99 | 0.24 | 1.18 | −1.02 | −2.02 | 0.21 | 1.16 |
| 41776_at | 0.78 | 1.72 | −1.06 | −2.09 | 0.20 | 1.15 | −1.24 | −2.36 | 0.42 | 1.34 |
| 36497_at | 0.24 | 1.18 | −1.35 | −2.54 | −0.87 | −1.82 | −3.25 | −9.50 | −0.33 | −1.26 |
| 37112_at | 1.05 | 2.07 | −4.93 | −30.45 | 2.04 | 4.12 | −3.49 | −11.21 | −0.37 | −1.29 |
| 41207_at | 1.20 | 2.30 | −0.99 | −1.99 | 0.88 | 1.83 | −1.11 | −2.16 | 0.81 | 1.75 |
| 38418_at | 0.04 | 1.03 | −1.13 | −2.19 | 0.10 | 1.07 | −1.34 | −2.53 | 0.51 | 1.43 |
| 2020_at | 0.19 | 1.14 | −1.10 | −2.14 | 0.15 | 1.11 | −1.24 | −2.35 | 0.55 | 1.47 |
| 39351_at | −0.37 | −1.29 | −0.99 | −1.99 | −0.49 | −1.41 | −1.32 | −2.50 | −0.60 | −1.51 |
| 41138_at | −0.48 | −1.39 | −1.51 | −2.85 | −0.17 | −1.12 | −0.98 | −1.97 | −0.05 | −1.03 |
| 32363_at | 0.98 | 1.97 | −2.49 | −5.63 | −0.43 | −1.35 | −3.53 | −11.58 | 0.51 | 1.43 |
| 40698_at | 0.18 | 1.14 | −1.21 | −2.31 | −0.10 | −1.07 | −3.09 | −8.50 | 0.22 | 1.16 |
| 34203_at | −0.33 | −1.25 | −3.42 | −10.67 | 0.70 | 1.63 | −3.33 | −10.06 | 1.22 | 2.33 |
| 39031_at | 3.84 | 14.35 | −6.97 | −125.51 | 2.88 | 7.34 | −7.36 | −163.71 | 2.79 | 6.93 |
| 32242_at | −0.28 | −1.21 | −2.96 | −7.75 | −0.63 | −1.55 | −5.49 | −44.79 | −0.73 | −1.66 |
| 32243_g_at | −0.01 | −1.01 | −2.70 | −6.51 | −0.73 | −1.66 | −5.49 | −44.79 | −0.25 | −1.19 |
| 859_at | 3.00 | 7.98 | −2.06 | −4.17 | 2.29 | 4.69 | −2.36 | −5.13 | 2.23 | 4.69 |
| 40071_at | 2.95 | 7.73 | −1.85 | −3.60 | 2.06 | 4.16 | −2.39 | −5.25 | 2.23 | 4.70 |
| 39140_at | 0.47 | 1.39 | −0.87 | −1.83 | −0.33 | −1.26 | −0.81 | −1.76 | −0.18 | −1.14 |
| 33337_at | −0.04 | −1.03 | −0.95 | −1.93 | −0.22 | −1.17 | −0.95 | −1.93 | −0.31 | −1.24 |
| 37000_at | 0.49 | 1.40 | −1.20 | −2.29 | 0.35 | 1.27 | −1.45 | −2.73 | 0.18 | 1.13 |
| 36861_at | 0.34 | 1.27 | −3.44 | −10.88 | 0.54 | 1.46 | −2.88 | −7.36 | 0.90 | 1.87 |
| 35977_at | −0.33 | −1.26 | −2.17 | −4.48 | 0.30 | 1.23 | −1.79 | −3.45 | 0.07 | 1.05 |
| 36133_at | 1.20 | 2.30 | −3.38 | −10.42 | −0.73 | −1.66 | −3.37 | −10.34 | 0.51 | 1.43 |
| 37600_at | −0.16 | −1.11 | −1.96 | −3.89 | −0.19 | −1.14 | −1.81 | −3.51 | 0.23 | 1.17 |
| 39098_at | −0.11 | −1.08 | −3.63 | −12.39 | −0.81 | −1.75 | −4.59 | −24.11 | 0.59 | 1.51 |
| 39861_at | −0.03 | −1.02 | −1.71 | −3.28 | 0.10 | 1.07 | −1.25 | −2.38 | −0.27 | −1.20 |
| 41385_at | −0.54 | −1.46 | −1.56 | −2.95 | −0.03 | −1.02 | −5.51 | −45.68 | 3.42 | 10.71 |
| 32148_at | −0.07 | −1.05 | −3.71 | −13.12 | 0.35 | 1.28 | −2.17 | −4.49 | 0.01 | 1.01 |
| 39038_at | −1.14 | −2.21 | −0.92 | −1.89 | −1.94 | −3.85 | −1.90 | −3.72 | −1.13 | −2.18 |
| 37743_at | −0.61 | −1.53 | −3.96 | −15.58 | 0.91 | 1.88 | −2.19 | −4.57 | 0.01 | 1.01 |
| 38651_at | −0.28 | −1.21 | −0.95 | −1.93 | −0.01 | −1.01 | −1.26 | −2.39 | −0.30 | −1.23 |
| 40468_at | 0.25 | 1.19 | −1.20 | −2.29 | 0.02 | 1.01 | −1.30 | −2.47 | −0.03 | −1.02 |
| 35785_at | 0.67 | 1.59 | −2.22 | −4.67 | 1.01 | 2.01 | −1.65 | −3.15 | 0.32 | 1.25 |
| 905_at | 0.19 | 1.14 | −0.63 | −1.55 | −0.15 | −1.11 | −0.67 | −1.59 | 0.10 | 1.07 |
| 38824_at | 1.90 | 3.74 | −1.51 | −2.85 | 1.03 | 2.05 | −2.47 | −5.52 | 1.28 | 2.42 |
| 39781_at | 0.79 | 1.73 | −1.31 | −2.48 | 0.16 | 1.12 | −2.35 | −5.08 | 0.66 | 1.58 |
| 38636_at | −0.06 | −1.04 | −2.63 | −6.18 | 0.12 | 1.09 | −2.16 | −4.46 | 0.16 | 1.12 |
| 35318_at | −0.89 | −1.85 | −1.02 | −2.02 | −0.56 | −1.48 | −1.78 | −3.42 | 0.06 | 1.04 |
| 36453_at | 0.08 | 1.06 | −4.76 | −27.13 | 0.04 | 1.03 | −3.33 | −10.04 | −0.55 | −1.46 |
| 41585_at | 0.16 | 1.12 | −2.20 | −4.61 | −0.01 | −1.01 | −2.61 | −6.09 | −0.42 | −1.34 |
| 32730_at | 2.35 | 5.10 | −1.90 | −3.72 | 1.71 | 3.28 | −3.93 | −15.24 | 3.18 | 9.09 |
| 38972_at | 0.40 | 1.32 | −2.50 | −5.67 | 0.00 | 1.00 | −1.24 | −2.36 | −0.47 | −1.39 |
| 35917_at | −0.86 | −1.82 | −0.88 | −1.84 | −1.03 | −2.05 | −1.11 | −2.16 | −1.89 | −3.71 |
| 34403_at | −0.13 | −1.09 | −3.59 | −12.01 | 0.50 | 1.42 | −3.21 | −9.23 | 0.40 | 1.32 |
| 33447_at | 1.12 | 2.17 | −1.90 | −3.72 | 0.89 | 1.86 | −1.26 | −2.39 | 0.08 | 1.05 |
| 36073_at | −0.59 | −1.51 | −4.20 | −18.40 | 2.45 | 5.46 | −4.35 | −20.44 | 3.44 | 10.84 |
| 38750_at | −1.80 | −3.48 | −3.76 | −13.50 | 0.39 | 1.31 | −5.28 | −38.76 | 0.07 | 1.05 |
| 41742_s_at | 0.83 | 1.77 | −1.85 | −3.60 | 0.70 | 1.63 | −1.09 | −2.13 | 0.34 | 1.26 |
| 32260_at | −0.15 | −1.11 | −1.34 | −2.53 | −0.35 | −1.27 | −1.65 | −3.13 | −0.14 | −1.11 |
| 40434_at | 0.98 | 1.97 | −2.27 | −4.82 | 0.84 | 1.79 | −5.38 | −41.74 | 1.74 | 3.34 |
| 35841_at | −0.05 | −1.03 | −1.13 | −2.18 | −0.31 | −1.24 | −1.70 | −3.25 | −0.19 | −1.14 |
| 503_at | 0.00 | 1.00 | −0.98 | −1.97 | −0.31 | −1.24 | −1.41 | −2.65 | −0.19 | −1.14 |
| 34797_at | 0.33 | 1.26 | −1.82 | −3.52 | −0.28 | −1.22 | −1.57 | −2.97 | −0.48 | −1.39 |
| 39366_at | −0.73 | −1.66 | −2.24 | −4.72 | −0.13 | −1.09 | −2.64 | −6.23 | −0.77 | −1.70 |
| 36533_at | 2.47 | 5.55 | −2.77 | −8.84 | 1.43 | 2.70 | −4.54 | −23.21 | −0.24 | −1.18 |
| 39244_at | 2.12 | 4.34 | −1.51 | −2.84 | 0.43 | 1.34 | −3.00 | −7.98 | 1.96 | 3.90 |
| 38264_at | 0.14 | 1.10 | −1.19 | −2.28 | 0.30 | 1.23 | −0.92 | −1.89 | −0.06 | −1.04 |
| 38331_at | 0.66 | 1.58 | −0.61 | −1.52 | 0.02 | 1.01 | −1.13 | −2.19 | 0.17 | 1.12 |
| 32827_at | −0.39 | −1.31 | −1.40 | −2.65 | −0.73 | −1.66 | −1.63 | −3.10 | −0.23 | −1.18 |
| 39338_at | 0.52 | 1.43 | −0.86 | −1.81 | −0.13 | −1.09 | −1.34 | −2.54 | 0.26 | 1.19 |
| 38138_at | 0.32 | 1.25 | −0.78 | −1.72 | −0.17 | −1.12 | −1.18 | −2.27 | 0.02 | 1.01 |
| 38087_s_at | 0.56 | 1.48 | −1.34 | −2.53 | −1.46 | −2.75 | −3.98 | −15.73 | 1.05 | 2.06 |
| 39775_at | −0.34 | −1.26 | −1.17 | −2.24 | −0.44 | −1.36 | −1.29 | −2.44 | −0.28 | −1.21 |
| 34993_at | −0.83 | −1.77 | −2.17 | −4.51 | −2.56 | −5.88 | −1.32 | −2.50 | −2.62 | −6.13 |
| 39260_at | 2.46 | 5.52 | −3.71 | −13.07 | 1.81 | 3.50 | −2.98 | −7.91 | 1.02 | 2.03 |
| 32574_at | −0.28 | −1.21 | −1.45 | −2.72 | −0.48 | −1.39 | −1.30 | −2.48 | −0.46 | −1.37 |
| 1686_g_at | 1.57 | 2.97 | −1.80 | −3.49 | 0.00 | 1.00 | −1.40 | −2.63 | 0.08 | 1.05 |
| 40419_at | 1.18 | 2.27 | −2.33 | −5.02 | 0.52 | 1.43 | −1.06 | −2.08 | 0.38 | 1.30 |
| 35832_at | −0.04 | −1.03 | −7.66 | −202.25 | 0.44 | 1.36 | −5.21 | −36.97 | −1.85 | −3.60 |
| 36931_at | −0.38 | −1.30 | −2.88 | −7.34 | −1.16 | −2.24 | −1.72 | −3.29 | −1.51 | −2.85 |
| 1596_g_at | 3.97 | 15.63 | −4.60 | −24.17 | −1.11 | −2.15 | −4.01 | −16.07 | −0.85 | −1.80 |
| 37643_at | 0.55 | 1.46 | −1.91 | −3.75 | −0.15 | −1.11 | −1.34 | −2.53 | −0.19 | −1.14 |
| 1441_s_at | 0.31 | 1.24 | −1.79 | −3.47 | 0.16 | 1.11 | −1.86 | −3.62 | 0.66 | 1.58 |
| 32313_at | −1.13 | −2.20 | −1.04 | −2.06 | −0.82 | −1.76 | −0.74 | −1.67 | −0.93 | −1.90 |
| 32314_g_at | −0.89 | −1.85 | −0.80 | −1.74 | −0.62 | −1.53 | −0.51 | −1.42 | −0.83 | −1.78 |
| 39331_at | 1.26 | 2.40 | −0.92 | −1.90 | 0.65 | 1.57 | −0.86 | −1.82 | 0.31 | 1.24 |
| 32533_s_at | 0.01 | 1.00 | −2.06 | −4.16 | −0.15 | −1.11 | −1.10 | −2.14 | −0.66 | −1.58 |
| 40147_at | −0.02 | −1.02 | −0.90 | −1.87 | 0.17 | 1.12 | −1.02 | −2.02 | 0.24 | 1.18 |
| 36170_at | 0.02 | 1.01 | −1.58 | −3.00 | 0.20 | 1.15 | −1.07 | −2.10 | −0.16 | −1.12 |
| 39170_at | −0.73 | −1.66 | −1.99 | −3.98 | −0.36 | −1.28 | −1.21 | −2.32 | −0.79 | −1.73 |
| 39162_at | 0.03 | 1.02 | −0.70 | −1.62 | −0.27 | −1.20 | −0.68 | −1.61 | −0.70 | −1.62 |

| | | | | | | | |
|---|---|---|---|---|---|---|---|
| | | | | | 041 HP Ave | 041 5-aza | N-UT Ave |
| | | | | | Fold | Ave Fold | Fold |
| Probe ID | Unigene | Locus ID | Symbol | Chromosome | Change | Change | Change |

(C) SA_D_Annotated_Averages

| | | | | | | | | | | |
|---|---|---|---|---|---|---|---|---|---|---|
| 37858_at | Hs.8769 | 83804 | BCMP1 | Xp11.0 | −0.82 | −1.77 | −0.88 | −1.87 | −0.83 | −1.55 |
| 32643_at | Hs.1681 | 2532 | GBE1 | 3p12.3 | −0.34 | −1.30 | −0.92 | −1.89 | 0.33 | 1.17 |
| 38348_at | Hs.235887 | 3275 | HRMTIL1 | 21q22.3 | 0.03 | 1.02 | −0.71 | −1.83 | 0.52 | 1.43 |
| 38065_at | Hs.4980 | 9079 | LDB2 | 4p18 | 0.02 | 1.76 | −0.85 | −1.83 | 1.06 | 2.08 |
| 34283_at | Hs.22907 | 703824 | LOC783824 | 18p13.12 | 0.15 | 1.11 | −1.28 | −2.42 | −0.37 | −1.30 |
| 34357_at | Hs.3343 | 78227 | PNGOH | 1p12 | 0.32 | 1.25 | −0.84 | −1.79 | 0.51 | 1.47 |

(D) SA_t_Annotated_Averages

| | | | | | | | | | | |
|---|---|---|---|---|---|---|---|---|---|---|
| 36589_at | Hs.75313 | 231 | AKR181 | 7q35 | −0.42 | −1.34 | 1.07 | 2.10 | −0.20 | −1.15 |
| 36688_at | Hs.75746 | 220 | ALDH1A3 | 15q28.3 | −1.42 | −2.67 | 1.50 | 2.63 | −1.18 | −2.26 |
| 39158_at | Hs.9734 | 22809 | ATFS | 19q13.3 | 0.13 | 1.10 | 2.10 | 4.30 | 0.06 | 1.04 |
| 1717_s_at | Hs.127799 | 330 | BIRC3 | 11q22 | −2.79 | −8.92 | 2.70 | 6.51 | 0.67 | 1.59 |
| 40385_at | Hs.75498 | 8384 | CCL20 | 2q33-q37 | 1.84 | 3.58 | 4.29 | 19.58 | −1.22 | −2.33 |
| 1274_s_at | Hs.423615 | 997 | CDC34 | 19p13.3 | 0.79 | 1.73 | 0.74 | 1.67 | 0.12 | 1.09 |
| 1211_s_at | Hs.155568 | 8738 | CRADD | 12q21.33-q23 | 0.92 | 1.89 | 1.47 | 2.77 | 0.88 | 1.84 |
| 33637_g_at | Hs.167379 | 1485 | CTAG1 | Xq28 | 0.73 | 1.85 | 2.97 | 7.82 | 0.69 | 1.82 |
| 33838_at | Hs.167379 | 1485 | CTAG1 | Xq28 | 0.23 | 1.17 | 4.34 | 20.28 | 0.34 | 1.27 |
| 37187_at | Hs.73765 | 2920 | CXCL2 | 4q21 | −2.75 | −5.73 | 3.70 | 13.01 | 2.49 | 3.82 |
| 34022_at | Hs.89690 | 2921 | CXC13 | 4q21 | −1.33 | −2.51 | 3.84 | 14.27 | 0.79 | 1.72 |
| 33410_at | Hs.164021 | 8372 | CXCL6 | 4q21 | −6.21 | −73.94 | 3.34 | 10.10 | 5.54 | 46.53 |
| 859_at | Hs.154634 | 1545 | CYP1B1 | 2p21 | −3.81 | −14.04 | 3.03 | 8.17 | −3.06 | −8.36 |
| 40071_at | Hs.154634 | 1545 | CYP1B1 | 2p21 | −3.47 | −11.04 | 1.81 | 3.75 | −3.03 | −6.16 |
| 33972_s_at | Hs.73078 | 1618 | DAZL | 3p24.3 | 0.51 | 1.42 | 6.35 | 81.28 | −1.02 | −2.02 |
| 33871_f_at | Hs.73078 | 1818 | DAZL | 3p24.3 | −0.34 | −1.27 | 8.08 | 67.57 | 0.89 | 1.55 |
| 529_at | Hs.2128 | 1847 | DUSP5 | 10q25 | −1.97 | −3.80 | 2.20 | 4.58 | 2.32 | 4.98 |
| 41193_at | Hs.180393 | 1848 | DUSP6 | 12q22-q23 | −2.08 | −4.21 | 1.96 | 3.90 | 1.74 | 3.34 |
| 38326_at | Hs.432132 | 50486 | G0S2 | 1q32.2-q41 | −0.86 | −1.82 | 2.72 | 6.58 | 0.14 | 1.10 |
| 1107_s_at | Hs.432233 | 9636 | G1P2 | 1p38.33 | −1.88 | −3.62 | 4.03 | 16.32 | 1.10 | 2.14 |
| 31960_f_at | Hs.367724 | 2574 | GAGE2 | Xp11.4-p11.2 | 2.68 | 6.42 | 7.52 | 182.91 | 0.46 | 1.38 |
| 33671_f_at | Hs.183199 | 2576 | GAGE4 | Xp11.4-p11.2 | 2.31 | 4.97 | 7.85 | 229.92 | 0.50 | 1.41 |
| 37085_f_at | Hs.378444 | 2577 | GAGE5 | Xp11.4-p11.2 | 1.41 | 2.65 | 7.44 | 173.63 | −0.42 | −1.33 |
| 31498_f_at | Hs.272484 | 2578 | GAGE6 | Xp11.4-p11.2 | 1.19 | 2.27 | 8.70 | 104.21 | 1.80 | 3.49 |
| 33680_f_at | Hs.278806 | 2579 | GAGE7 | Xp11.2-p11.2 | 1.42 | 2.57 | 6.94 | 122.93 | −0.72 | −1.65 |
| 31954_f_at | Hs.251677 | 28748 | GAGE78 | Xp11.4-p11.2 | 1.98 | 3.94 | 7.38 | 166.18 | 0.32 | 1.24 |
| 31595_s_at | Hs.76057 | 2582 | GALE | 1p36-p35 | −1.23 | −2.35 | 1.06 | 2.09 | 0.45 | 1.38 |
| 37944_at | Hs.86724 | 2543 | GCH1 | 14q22.1-q22.2 | −0.93 | −1.91 | 4.33 | 20.04 | 0.71 | 1.64 |
| 34311_at | Hs.28988 | 2745 | GLRX | 5q14 | −2.03 | −4.07 | 1.37 | 2.58 | −0.39 | −1.31 |
| 37483_at | Hs.116753 | 9734 | HDAC9 | 7p21p15 | −1.88 | −3.87 | 2.47 | 5.54 | 0.28 | 1.21 |
| 37018_at | Hs.7644 | 3006 | HIST1H1C | 6p21.3 | −0.75 | −1.68 | 1.18 | 2.27 | 0.78 | 1.71 |
| 32980_f_at | Hs.356901 | 8347 | HIST1H2BC | 6p21.3 | 0.01 | 1.00 | 1.55 | 2.93 | 0.72 | 1.64 |
| 31522_f_at | Hs.182137 | 8343 | HIST1H2BF | 6p21.3 | 0.06 | 1.04 | 1.72 | 3.29 | 0.73 | 1.65 |
| 31524_f_at | Hs.182140 | 8346 | HIST1H2BI | 6p21.3 | 0.18 | 1.12 | 1.80 | 3.47 | 0.69 | 1.61 |
| 153_f_at | Hs.285735 | 8970 | HIST1H2BJ | 6p21.33 | −0.89 | −1.81 | 1.68 | 3.19 | 0.28 | 1.22 |
| 36347_f_at | Hs.154576 | 8341 | HIST1H2BN | 6p22-p21.3 | −0.14 | −1.10 | 2.04 | 4.11 | 1.02 | 2.02 |
| 34984_at | Hs.143042 | 8351 | HIST1H3D | 6p21.3 | −0.59 | −1.50 | 2.33 | 5.03 | 1.87 | 3.65 |
| 288_at | Hs.417332 | 8337 | HIST2H2AA | 1q21.2 | −1.45 | −2.73 | 2.50 | 5.66 | 0.12 | 1.09 |
| 32609_at | Hs.417332 | 8337 | HIST2H2AA | 1q21.2 | −2.51 | −5.69 | 2.55 | 5.66 | −0.30 | −1.23 |
| 1016_s_at | Hs.25954 | 3598 | IL13RA2 | Xq13.1-q28 | −2.43 | −5.40 | 2.01 | 4.03 | 1.69 | 3.23 |
| 39402_at | Hs.128256 | 3533 | IL18 | 2q14 | −0.32 | −1.25 | 3.66 | 12.66 | 3.12 | 8.66 |
| 1520_s_at | Hs.128256 | 3553 | IL18 | 2q14 | −0.38 | −1.30 | 4.58 | 23.92 | 2.95 | 7.71 |
| 38299_at | Hs.93913 | 3569 | IL5 | 7p21 | 0.83 | 1.78 | 5.80 | 48.39 | 1.55 | 2.93 |
| 35372_r_at | Hs.624 | 3576 | IL8 | 4q13-q21 | −2.24 | −4.73 | 3.42 | 10.72 | 2.18 | 4.56 |
| 33304_at | Hs.183487 | 3669 | ISG20 | 15q26 | −1.51 | −2.85 | 3.62 | 12.28 | 2.12 | 4.35 |
| 41481_at | Hs.27198 | 3673 | ITGA2 | 5q23-q31 | −3.00 | −7.97 | 3.94 | 15.30 | 1.45 | 2.77 |
| 41179_at | Hs.179946 | 22838 | KIAA1100 | 5q35.3 | −0.18 | 1.14 | 0.85 | 1.60 | 1.04 | 2.05 |
| 32730_at | Hs.173094 | 85453 | KIAA1750 | 8q22.1 | −4.80 | −27.76 | 1.53 | 2.88 | −4.23 | −18.81 |
| 35768_at | Hs.406013 | 3875 | KRT18 | 12q13 | −2.03 | −4.08 | 4.05 | 16.58 | −1.99 | −3.97 |
| 36288_at | Hs.32952 | 3887 | KRTHB1 | 12q13 | 0.53 | 1.45 | 5.32 | 39.85 | 0.41 | 1.33 |
| 36929_at | Hs.75317 | 3914 | LAMB3 | 1q32 | 0.23 | 1.18 | 2.06 | 4.18 | 1.87 | 3.65 |
| 37754_at | Hs.79339 | 3959 | LGAL93BP | 17q25 | −0.22 | −1.17 | 4.25 | 19.05 | −0.35 | −1.28 |
| 38062_at | Hs.49587 | 9404 | LPXN | 11q12.1 | −0.79 | −1.73 | 0.83 | 1.54 | −0.22 | −1.18 |
| 36711_at | Hs.51305 | 23764 | MAFF | 22q13.1 | −1.62 | −3.06 | 1.99 | 3.98 | 0.33 | 1.26 |
| 32428_f_at | Hs.72879 | 4100 | MAGEA1 | Xq28 | 1.84 | 3.58 | 3.66 | 12.87 | 0.18 | 1.12 |
| 36302_f_at | Hs.37107 | 4103 | MAGEA4 | Xq28 | 1.42 | 2.67 | 4.73 | 28.57 | 0.06 | 1.04 |
| 35097_at | Hs.113824 | 4113 | MAGEB2 | Xq21.3 | 4.59 | 24.03 | 5.06 | 33.40 | −1.15 | −2.22 |
| 39370_at | Hs.121849 | 81631 | MAP1LC3B | 16q24.2 | −0.66 | −1.58 | 0.73 | 1.68 | −1.31 | −2.47 |
| 38428_at | Hs.83169 | 4312 | MUP1 | 11q22.3 | −3.26 | −9.58 | 2.18 | 4.52 | −0.09 | −1.06 |
| 35138_at | Hs.25010 | 55918 | NXT2 | Xq22.3 | 0.43 | 1.34 | 1.35 | 2.55 | 0.80 | 1.74 |
| 33649_at | Hs.239138 | 10113 | PBEF | 7q22.1 | 0.02 | 1.02 | 2.32 | 4.99 | 1.29 | 2.44 |
| 1890_at | Hs.296639 | 9518 | PLAB | 19p13.1-13.2 | 0.30 | 1.23 | 1.29 | 2.45 | −0.93 | −1.91 |
| 37310_at | Hs.77274 | 3328 | PLAU | 10q24 | −4.17 | −17.94 | 1.99 | 3.97 | 0.27 | 1.20 |
| 41048_at | Hs.96 | 5366 | PMAIP1 | 18q21.31 | −1.16 | −2.23 | 2.10 | 4.30 | 0.80 | 1.74 |
| 38886_at | Hs.1050 | 9267 | PSCD1 | 17q25 | −0.71 | −1.63 | 1.16 | 2.23 | 0.11 | 1.08 |
| 41184_s_at | Hs.180062 | 5696 | PSMB8 | 6p21.3 | −0.64 | −1.55 | 1.82 | 3.52 | 1.43 | 2.69 |
| 34304_s_at | Hs.20491 | 6303 | SAT | Xp22.1 | −0.62 | −1.54 | 1.20 | 2.31 | −0.02 | −1.02 |
| 35488_at | Hs.19312 | 6817 | SNAPC1 | 14q22 | 0.32 | 1.25 | 1.21 | 2.31 | 0.02 | 1.02 |
| 40898_at | Hs.182248 | 8878 | SO5TM1 | 5q35 | −0.70 | −1.62 | 1.07 | 2.10 | 0.02 | 1.01 |
| 36409_f_at | Hs.289105 | 6757 | SSX2 | Xp11.23-p11.7 | 0.68 | 1.60 | 5.07 | 33.87 | 0.08 | 1.06 |
| 33855_f_at | Hs.178749 | 10214 | SSX3 | Xp11.23 | 0.97 | 1.96 | 1.89 | 3.71 | 1.10 | 2.15 |
| 35950_at | Hs.278632 | 6759 | SSX4 | Xp11.23 | 0.43 | 1.35 | 1.54 | 2.91 | 0.41 | 1.33 |
| 32134_at | Hs.165986 | 26138 | TES | 7q31.2 | −4.91 | −30.13 | 2.89 | 7.40 | −4.06 | −16.70 |
| 37388_at | Hs.205944 | 7980 | TFP12 | 7q22 | −1.40 | −2.64 | 4.30 | 19.72 | 1.57 | 2.97 |
| 231_at | Hs.75307 | 7052 | TGM2 | 20q12 | −1.93 | −3.80 | 3.20 | 9.18 | 1.46 | 2.74 |
| 38404_s_at | Hs.75307 | 7052 | TGM2 | 20q12 | −6.23 | −75.15 | 7.14 | 140.88 | 1.59 | 3.00 |
| 1693_s_at | Hs.5831 | 7076 | TIMP1 | Xp11.3-p11.23 | −0.39 | −1.31 | 0.93 | 1.90 | −1.25 | −2.37 |
| 595_at | Hs.211600 | 7128 | TNFA1P3 | 6q23 | −1.61 | −3.05 | 1.07 | 2.09 | −0.33 | −1.25 |
| 34892_at | Hs.31233 | 8793 | TNFRSF108 | 8p22-p21 | −0.07 | −1.05 | 0.93 | 1.91 | −1.07 | −2.09 |
| 40090_g_at | Hs.155020 | 114049 | WBSCR22 | | 0.30 | 1.23 | 1.40 | 2.64 | −0.10 | −1.07 |
| 1173_s_at | | | | | −0.37 | −1.29 | 1.54 | 2.91 | −0.04 | −1.03 |
| 39525_at | Hs.351597 | | | | 0.368 | 1.28 | 1.35 | 2.55 | −0.70 | −1.62 |
| 189_at | | | | | −0.72 | −1.65 | 1.79 | 3.45 | −0.27 | −1.21 |
| 39420_at | Hs.408544 | | | | −0.71 | −1.83 | 1.09 | 2.12 | −0.16 | −1.11 |
| 126_s_at | | | | | 0.02 | 1.01 | 3.04 | 8.23 | −1.10 | −2.14 |

| | | | | | |
|---|---|---|---|---|---|
| | N-5aza Ave | 1-UT Ave | 1-5aza Ave | 10-UT Ave | 10-5aza |
| | Fold | Fold | Fold | Fold | Ave Fold |
| Probe ID | Change | Change | Change | Change | Change |

| (C) SA_D_Annotated_Averages | | | | | | | | | | |
|---|---|---|---|---|---|---|---|---|---|---|
| 37858_at | −1.54 | −2.90 | −2.12 | −4.34 | −1.24 | −2.37 | −1.89 | −3.23 | −1.40 | −2.84 |
| 32643_at | −0.89 | −1.85 | −0.37 | −1.38 | −0.78 | −1.89 | 0.15 | 1.11 | −0.812 | −1.77 |
| 38348_at | −0.73 | −1.86 | −0.79 | −1.88 | −0.70 | −1.02 | −0.20 | −1.15 | −0.76 | −1.70 |
| 38065_at | −1.50 | −3.83 | 3.01 | 4.01 | −1.85 | −3.68 | 1.02 | 3.03 | −0.87 | −1.83 |
| 34283_at | −1.30 | −4.92 | −0.32 | −1.25 | −1.79 | −3.48 | 0.29 | 1.32 | −3.16 | −4.34 |
| 34357_at | −0.83 | −1.90 | 0.49 | 1.40 | −0.59 | −1.51 | 0.57 | 1.43 | −0.93 | −1.80 |
| (D) SA_t_Annotated_Averages | | | | | | | | | | |
| 36589_at | 0.62 | 1.54 | 0.48 | 1.39 | 0.41 | 1.33 | −0.17 | −1.13 | 0.55 | 1.47 |
| 36688_at | 1.70 | 3.24 | −3.00 | −8.00 | 1.19 | 2.29 | −2.15 | −4.42 | 1.94 | 3.83 |
| 39158_at | 2.43 | 5.37 | 0.29 | 1.22 | 2.43 | 5.41 | 0.80 | 1.74 | 1.98 | 3.94 |
| 1717_s_at | 3.22 | 9.34 | 0.43 | 1.34 | 3.21 | 9.27 | 0.48 | 1.39 | 2.73 | 6.61 |
| 40385_at | 6.57 | 117.24 | −1.20 | −2.30 | 7.09 | 136.13 | −1.09 | −2.12 | 6.15 | 70.79 |
| 1274_s_at | 0.85 | 1.80 | −0.28 | −1.21 | 1.03 | 2.04 | 0.32 | 1.24 | 0.95 | 1.94 |
| 1211_s_at | 1.64 | 3.12 | −0.54 | −1.46 | 1.91 | 3.72 | 0.24 | 1.18 | 1.81 | 3.05 |
| 33637_g_at | 3.39 | 10.48 | 1.75 | 3.37 | 1.81 | 3.51 | 1.10 | 2.14 | 2.45 | 5.46 |
| 33838_at | 4.86 | 29.13 | −0.02 | −1.02 | 4.24 | 18.84 | 0.79 | 1.73 | 3.48 | 11.03 |
| 37187_at | 3.08 | 8.43 | 3.46 | 11.00 | 3.27 | 9.82 | 2.98 | 7.76 | 2.85 | 7.19 |
| 34022_at | 2.48 | 5.58 | 1.59 | 3.02 | 4.72 | 18.58 | 1.14 | 2.20 | 2.96 | 7.78 |
| 33410_at | 1.44 | 2.77 | 8.97 | 125.51 | 2.83 | 7.60 | 5.50 | 45.38 | 1.72 | 3.29 |
| 859_at | 3.00 | 7.98 | −2.06 | −4.17 | 2.29 | 4.68 | −2.36 | −5.13 | 2.23 | 4.69 |
| 40071_at | 2.95 | 7.73 | −1.85 | −3.60 | 2.06 | 4.16 | −2.38 | −5.25 | 2.23 | 4.70 |
| 33972_s_at | 6.26 | 76.65 | −0.28 | −1.21 | 5.31 | 39.70 | 0.05 | 1.03 | 5.68 | 51.35 |
| 33871_f_at | 5.64 | 49.90 | −0.50 | −1.41 | 5.92 | 60.45 | 0.43 | 1.35 | 5.71 | 52.47 |
| 529_at | 1.20 | 2.30 | 1.10 | 2.14 | 1.79 | 3.47 | 0.38 | 1.29 | 1.41 | 2.68 |
| 41193_at | 1.54 | 2.92 | 2.01 | 4.02 | 1.49 | 2.82 | 0.67 | 1.59 | 2.30 | 4.91 |
| 38326_at | 3.41 | 10.52 | 3.75 | 13.45 | 1.42 | 2.68 | 1.39 | 2.62 | 2.90 | 7.47 |
| 1107_s_at | 2.27 | 4.81 | −1.05 | −2.07 | 0.97 | 1.96 | −1.01 | −2.02 | 1.79 | 3.45 |
| 31960_f_at | 7.78 | 220.30 | 7.84 | 199.24 | 1.38 | 2.61 | 0.95 | 1.93 | 7.03 | 130.79 |
| 33671_f_at | 7.40 | 185.38 | 8.23 | 300.59 | 1.40 | 2.65 | −0.01 | −1.01 | 7.94 | 245.00 |
| 37085_f_at | 7.58 | 191.34 | 7.30 | 157.04 | 1.37 | 2.58 | 1.03 | 2.03 | 6.28 | 77.59 |
| 31498_f_at | 6.44 | 88.76 | 8.04 | 263.81 | 1.40 | 2.65 | 1.36 | 2.57 | 7.56 | 168.83 |
| 33680_f_at | 7.44 | 173.24 | 7.03 | 130.84 | 1.40 | 2.64 | 0.16 | 1.11 | 6.79 | 110.32 |
| 31954_f_at | 8.35 | 326.79 | 8.76 | 439.59 | 1.44 | 2.72 | 0.67 | 1.53 | 7.47 | 176.88 |
| 31595_s_at | 1.27 | 2.41 | 0.64 | 1.56 | 1.40 | 2.65 | −0.54 | −1.45 | 2.42 | 5.37 |
| 37944_at | 2.53 | 5.79 | 0.74 | 1.87 | 2.80 | 8.97 | 1.37 | 2.58 | 2.11 | 4.30 |
| 34311_at | 1.29 | 2.44 | −1.96 | −3.90 | 1.72 | 3.30 | −1.71 | −3.27 | 1.33 | 2.52 |
| 37483_at | 0.88 | 1.81 | 0.24 | 1.18 | 1.47 | 2.78 | 0.43 | 1.34 | 1.27 | 2.41 |
| 37018_at | 1.95 | 3.85 | 2.43 | 5.39 | 1.83 | 3.56 | 0.30 | 1.23 | 2.38 | 5.26 |
| 32980_f_at | 1.48 | 2.79 | 1.48 | 2.75 | 1.33 | 2.52 | 0.11 | 1.08 | 1.28 | 2.39 |
| 31522_f_at | 1.85 | 3.59 | 1.73 | 3.31 | 1.48 | 2.78 | −0.17 | −1.13 | 1.68 | 3.21 |
| 31524_f_at | 1.46 | 2.75 | 1.85 | 3.61 | 1.05 | 2.07 | 0.26 | 1.20 | 1.11 | 2.15 |
| 153_f_at | 1.77 | 3.42 | 1.76 | 3.39 | 1.59 | 3.02 | −0.82 | −1.77 | 2.58 | 5.98 |
| 36347_f_at | 1.74 | 3.35 | 2.28 | 4.85 | 1.20 | 2.30 | 0.06 | 1.04 | 2.03 | 4.10 |
| 34984_at | 2.86 | 7.28 | 3.41 | 10.59 | 2.40 | 5.29 | −0.43 | −1.35 | 4.20 | 18.42 |
| 288_at | 2.90 | 7.49 | 0.68 | 1.60 | 2.18 | 4.52 | −1.02 | −2.03 | 2.23 | 4.71 |
| 32609_at | 3.25 | 9.51 | 0.30 | 1.23 | 2.46 | 5.52 | −1.85 | −3.81 | 2.78 | 6.68 |
| 1016_s_at | 3.40 | 10.59 | 2.34 | 5.07 | 4.06 | 16.71 | 0.74 | 1.87 | 3.78 | 13.71 |
| 39402_at | 2.04 | 4.11 | 0.96 | 1.94 | 3.87 | 14.61 | 2.06 | 4.18 | 2.25 | 4.75 |
| 1520_s_at | 2.41 | 5.33 | 0.81 | 1.52 | 4.68 | 25.81 | 1.53 | 2.68 | 3.20 | 9.20 |
| 38299_at | 3.46 | 10.99 | 0.52 | 1.44 | 3.87 | 14.63 | 3.01 | 8.05 | 1.71 | 3.27 |
| 35372_r_at | 2.61 | 5.11 | 1.65 | 3.13 | 4.01 | 16.09 | 2.80 | 6.98 | 2.77 | 6.80 |
| 33304_at | 2.23 | 4.68 | 1.11 | 2.16 | 1.17 | 2.26 | 1.13 | 2.19 | 1.32 | 2.49 |
| 41481_at | 2.33 | 5.04 | 2.37 | 5.13 | 1.57 | 2.97 | −0.06 | −1.04 | 3.74 | 13.33 |
| 41179_at | 0.81 | 1.76 | 0.01 | 1.01 | 1.04 | 2.06 | −0.07 | −1.05 | 1.38 | 2.57 |
| 32730_at | 2.35 | 5.10 | −1.90 | −3.72 | 1.71 | 3.28 | −3.93 | −15.24 | 3.18 | 9.09 |
| 35768_at | 4.61 | 24.40 | 0.30 | 1.23 | 1.96 | 3.90 | −0.20 | −1.15 | 2.48 | 5.58 |
| 36288_at | 6.27 | 77.11 | −0.01 | −1.00 | 5.03 | 32.87 | 1.20 | 2.29 | 5.51 | 45.57 |
| 36929_at | 1.33 | 2.52 | 1.35 | 2.55 | 1.90 | 3.74 | 1.42 | 2.87 | 2.50 | 3.87 |
| 37754_at | 2.71 | 6.56 | 0.25 | 1.19 | 1.02 | 2.03 | −1.28 | −2.42 | 2.84 | 6.23 |
| 38062_at | 0.79 | 1.73 | −0.28 | −1.21 | 1.18 | 2.28 | 0.25 | 1.19 | 0.92 | 1.89 |
| 36711_at | 1.38 | 2.57 | −0.64 | −1.55 | 2.23 | 4.70 | −0.54 | −1.48 | 2.14 | 4.42 |
| 32428_f_at | 4.56 | 23.62 | 3.86 | 14.54 | 1.39 | 2.82 | 0.45 | 1.36 | 4.54 | 23.19 |
| 36302_f_at | 5.71 | 52.51 | 1.53 | 2.59 | 3.69 | 12.91 | 0.18 | 1.13 | 4.57 | 23.77 |
| 35097_at | 8.95 | 124.02 | 2.80 | 8.95 | 3.39 | 10.45 | −0.79 | −1.73 | 5.62 | 49.18 |
| 39370_at | 1.32 | 2.50 | −1.01 | −2.01 | 1.65 | 3.13 | −0.43 | −1.35 | 0.06 | 1.82 |
| 38428_at | 2.26 | 4.80 | −0.10 | −1.07 | 3.16 | 6.91 | −0.95 | −1.93 | 2.65 | 6.27 |
| 35138_at | 1.45 | 2.72 | 1.49 | 2.81 | 1.73 | 3.32 | 0.28 | 1.21 | 1.40 | 7.64 |
| 33649_at | 1.21 | 2.32 | 1.33 | 2.51 | 2.17 | 4.49 | 1.68 | 3.21 | 1.85 | 3.61 |
| 1890_at | 1.81 | 3.51 | −0.77 | −1.70 | 2.50 | 5.67 | −0.57 | −1.49 | 2.22 | 4.66 |
| 37310_at | 2.21 | 4.61 | 0.36 | 1.28 | 2.41 | 5.30 | −0.35 | −1.27 | 2.53 | 5.79 |
| 41048_at | 1.87 | 3.17 | 1.32 | 2.50 | 1.55 | 2.93 | 1.18 | 2.26 | 2.26 | 4.79 |
| 38885_at | 0.88 | 1.84 | −0.45 | −1.38 | 1.09 | 2.12 | 0.15 | 1.12 | 0.98 | 1.97 |
| 41184_s_at | 0.55 | 1.58 | 0.21 | 1.15 | 0.77 | 1.71 | 0.07 | 1.05 | 0.82 | 1.76 |
| 34304_s_at | 0.95 | 1.93 | 0.18 | 1.12 | 1.29 | 2.45 | 0.43 | 1.34 | 0.95 | 1.93 |
| 35488_at | 1.28 | 2.44 | 0.02 | 1.02 | 1.58 | 3.00 | −0.40 | −1.32 | 1.90 | 3.74 |
| 40898_at | 1.08 | 2.12 | −0.36 | −1.28 | 1.54 | 2.90 | −0.10 | −1.07 | 0.94 | 1.92 |
| 36409_f_at | 6.34 | 81.07 | 4.74 | 28.89 | 7.96 | 7.79 | −0.14 | −1.10 | 8.15 | 283.83 |
| 33855_f_at | 1.36 | 2.56 | 2.33 | 5.04 | 2.54 | 5.82 | 0.23 | 1.17 | 4.85 | 25.18 |
| 35950_at | 2.10 | 4.30 | 2.99 | 7.94 | 1.75 | 3.36 | 0.49 | 1.40 | 3.41 | 10.66 |
| 32134_at | 2.36 | 5.13 | −0.58 | −1.49 | 0.94 | 1.92 | −0.64 | −1.56 | 0.85 | 1.81 |
| 37388_at | 3.07 | 8.39 | 1.50 | 2.83 | 3.43 | 10.81 | −0.26 | −1.19 | 5.14 | 35.32 |
| 231_at | 2.03 | 4.07 | 1.72 | 3.29 | 1.83 | 3.57 | −0.23 | −1.17 | 3.60 | 12.11 |
| 38404_s_at | 2.84 | 6.22 | 1.82 | 3.53 | 2.24 | 4.72 | −0.10 | −1.07 | 3.71 | 13.05 |
| 1693_s_at | 1.06 | 2.09 | −0.67 | −1.59 | 0.77 | 1.71 | −0.74 | −1.68 | 0.57 | 1.48 |
| 595_at | 1.49 | 2.81 | −0.97 | −1.95 | 2.00 | 3.99 | −0.04 | −1.03 | 1.41 | 2.66 |
| 34892_at | 1.09 | 2.12 | −0.56 | −1.47 | 1.09 | 2.13 | −0.70 | −1.62 | 1.26 | 2.39 |
| 40090_g_at | 1.03 | 2.05 | 0.99 | 1.98 | 0.89 | 1.62 | 0.50 | 1.41 | 1.15 | 2.21 |
| 1173_s_at | 1.13 | 2.19 | 0.37 | 1.29 | 1.22 | 2.34 | 0.46 | 1.37 | 0.86 | 1.82 |
| 39525_at | 1.14 | 2.21 | −0.72 | −1.65 | 1.17 | 2.25 | −0.50 | −1.41 | 0.91 | 1.88 |
| 189_at | 1.04 | 2.06 | 0.34 | 1.27 | 0.87 | 1.83 | 0.42 | 1.34 | 1.03 | 2.05 |
| 39420_at | 1.04 | 2.08 | −0.84 | −1.79 | 2.20 | 4.59 | −0.03 | −1.02 | 1.23 | 2.35 |
| 126_s_at | 6.33 | 80.33 | 3.47 | 11.08 | 3.78 | 13.78 | 0.61 | 1.75 | 7.81 | 195.21 |

### Example 4

Materials adn Methods

Cell Culture and Genotyping

The cell lines MDAH041 (p53 frameshift mutation) and MDAH087 (p53 missense point mutation) were derived from primary fibroblasts by skin biopsy from a female and male patient, respectively, with LFS (Bischoff et al. 1990). Four independent, spontaneously immortalized LFS cell lines were developed: one immortal cell line from MDAH041, and three independent immortal cell lines derived from MDAH087 (MDAH087-1, MDAH087-10 and MDAH087-N) (Gollahon et al. 1998). All the cells were cultured at 37° C. in 10% humidified CO₂ in Modified Eagles Medium (Gibco BRL, MD, USA) with 10% fetal bovine serum and 500 units/ml penicillin, 100 μg/ml streptomycin. The appropriate regions in the gene p53 containing the mutation were sequenced in the precrisis and immortal cell lines to confirm heterozygosity in the precrisis cell lines, and loss of the remaining wild-type p53 mutation in the immortal cell lines.

5-aza-dC Treatment, RNA Isolation and the Affymetrix Microarray Assays

Precrisis and immortal MDAH041 and MDAH87 fibroblasts were treated with 5-aza-dC as described (Kulaeva et al. 2003). Total RNA was extracted using the QIAGEN RNeasy Kit (QIAGEN, Inc., Valencia, Calif.). cRNA preparation and oligonucleotide analysis was performed in accordance with Affymetrix protocols. cRNA was hybridized to Affymetrix HGU95Av2 arrays (Affymetrix, Santa Clara, Calif., USA), which contains 12,625 probes.

Analysis of Microarray Data

Microarray experiments on MDAH087 were performed using the Affymetrix HGU95Av2 GeneChip® containing 12,625 probes. Three RNA preparations from MDAH087-N, MDAH087-1 and from MDAH087-10 were each compared with two RNA preparations from MDAH087-PC cells, individually. Three RNA preparations from 5-aza-dC treated MDAH087-N, MDAH087-1 and MDAH087-10 were each compared with RNA preparations from the corresponding untreated immortal MDAH087 cells separately. All the pairings of the comparisons were considered. Microarray data on MDAH041-PC, MDAH041 immortal and MDAH041 5-aza-dC treated cells was used in the microarray analysis performed in this study. Affymetrix DMT version 5 (Affymetrix, Santa Clara, Calif., USA) was used to select genes with increased expression. Probes with a detection call of present or marginal α1=0.00 and α2=0.06), and had a change call of increase or marginal increase (Change p-value, γ1=0.0025 and γ2=0.003) in all four immortal LFS cell lines, irrespective of fold change, in >65% of the chip comparisons, were identified as increased. Genes with decreased expression were similarly detected, but decreased probes with a detection call of absent were also included.

Identification of CpG Islands in Genes

To detect CpG islands in the promoter region of genes, there were identified −1000 bp to +500 bp, and '1500 bp to +200 bp of the transcription start site using UCSC Golden Path, http://qenome.ucsc.edu. MethPrimer (http:H/mail.ucsf.edu/˜urolab/methprimer/index1.html) was then used to identify CpG islands. The program analyzes windows of 100 base pairs with each subsequent window shifting 1 base pair over from the prior window. To determine if there is a CpG island for each window the program calculates the observed ratio of C plus G to CpG, and minimum average percentage G plus C; the default values were used for these parameters, >0.6 and >50, respectively.

Quantization of Gene Expression bv Q-RT-PCR

Two μg total RNA was reverse transcribed into cDNA using Superscript II (Invitrogen, Carlsbad, Calif.). Q-RT-PCR was performed using the SYBR Green PCR Detection Kit (PE Biosystems, Warrington, United Kingdom) and run on the ABI 5700 Sequence Detection System (Applied Biosystems, Foster City, Calif.). Primer Express Program (Applied Biosystems, Foster City, Calif.) was used to design primers for Q-RT PCR (Table 11). The relative fold change, 2^{-ΔΔc}_{T,} where, ΔΔC_{T}=(C_{T Gene of interest}−C_{T GAPDH})ₑₓₚₑᵣᵢₘₑₙₜ−(C_{T Gene of interest}−C_{T GAPDH})_{control}), of the transcript of interest was determined by comparing it to the reference gene transcript, GAPDH (Schmittgen et al. 2000). If the relative fold change was between 0 and 1, then the fold change was calculated by dividing −1 by the relative fold change. Fold changes of replicates were averaged.

Western Blot Analysis

Total cellular protein was harvested from untreated and 5-aza-dC treated LFS cells. Extracts were prepared using PBS-TDS (10 mM Na₂HPO₄, 154 mM NaCl, 12 mM cholic acid, sodium salt, 3.5 mM SDS, 31 mM sodium azide, 1 mM sodium fluoride, 1% Triton X-100) and 1% protease inhibitor cocktail (Sigma, St. Louis, Mo.). Lysates were incubated on ice for 30 minutes followed by centrifugation at 10,000×g. Protein was quantitated using the Bradford Reagent (Sigma, St. Louis, Mo.). Equal amounts of protein were electrophoresed in an appropriate percentage SDS-polyacrylamide gel (SDS-PAGE) and transferred to nitrocellulose membranes. The membranes were incubated with antibodies as indicated. Antibodies to the following molecules were used: p21^{CIP1/WAF1} (Upstate Biotechnologies, Lake Placid, N.Y.), p16^{INK4a} (PharMingen, San Diego, Calif.), α-Tubulin (Sigma, St. Louis, Mo.), and p53, STAT1α, IGFBP3, IGFBP4 and IGFBPrP1 were from Santa Cruz (Santa Cruz, Calif.). The western blots were then incubated with a horseradish peroxidase-conjugated secondary and developed using SuperSignal West Pico (Pierce, Rockford, Ill.). As a control, parallel western blots were probed with α-Tubulin.

Clustering Analysis

All the gene expression data on HGU95Av2 were processed as previously described and used for the hierarchical clustering analysis implemented in GeneSight, version 3.2.6 (Biodiscovery, Los Angeles, Calif.). Euclidean distance was used for measuring similarities between two genes or samples, and complete linkage was used for clustering. For each of the four immortal LFS cell lines there were two comparisons, immortal cells versus precrisis cells, and 5-aza-dC treated immortal cells versus untreated immortal cells. Two-sided hierarchical analysis was carried out to determine the similarities of the four immortal LFS cell lines across the whole gene expression data.

Multidimensional scaling is an alternative way to present the data in low dimension space. Multidimensional scaling analysis was performed using BRB-ArrayTools version 3.2 beta to plot the data in three dimensions. The same comparisons and parameters used for hierarchical clustering were also used for multidimensional scaling analysis.

Gene Ontology Analysis

GoMiner (version 122) (Zeeberg et al. 2003) was used to annotate the gene expression data with GO categories. The entire HGU95Av2 GeneChip® probe set was the reference. Four experiment genes lists were analyzed: genes that were up- and downregulated during immortalization in all four immortal LFS cell lines (A and B in Table 7), and genes that were up- and downregulated after 5-aza-dC treatment in all four immortal LFS cell lines (C and D in Table 7). The probes from the lists were first converted to unique gene symbols using NetAffx, the Affymetrix online database (Build # 166) (Liu et al. 2003), and then the unique list of gene symbols were analyzed by GoMiner. The 8,487 unique gene symbols on the HGU95Av2 GeneChip® were linked to 6,020 GO categories. The one-sided Fisher's exact test p-values calculated by GoMiner were used to evaluate the statistical significance of changes for a GO category. The p-values for the first layer GO categories were converted to −log₁₀(p-value) and graphed (FIG. 6).

When large numbers of trials are tested, a multiple comparisons problem occurs because there is an accumulation of type I error for an individual test on the level of the whole experiment. False discovery rate (FDR) is one of the least conservative correction methods and allows for tests of variables with some dependencies (Benjamini 1995; Benjamini 2001). FDR was used to correct for type I error for an individual test to achieve an acceptable type I error at the level of the whole experiment. The corrected p-values were calculated as P×i/R (P is the original p-value calculated from GoMiner; i is the index for increasing-sorted p-value; R is the total GO categories) for each GO category.

Chromosome Ideogram

The chromosome region and cytogenetic location for the genes was obtained using NetAffx annotation file for HGU95Av2, which used NCBI genome version 34. For those genes that did not have a chromosome region or cytogenetic location associated with them, and genes for which there was a discrepancy in the chromosome region and cytogenetic region, chromosome information was obtained using NCBI and GeneLoc. A modified version of colored Chromosomes.pl (Böhringer S 2002) was then used to generate the chromosome ideograms.

Comparison of Data with Interferon, Imprinting Genes and p53 Regulated Genes

Common gene lists (Table 7) were searched for IFN regulated genes, p53 regulated genes and for imprinted genes. The list of 1,061 IFN regulated genes was prepared from the IFN stimulated gene database of IFN-α and IFN-β inducible genes (http://www.lerner.ccf.orq/labs/williams/der.html) (Der et al. 1998) and from the IFN regulated genes identified by Dr. Leaman, University of Toledo. The imprinted genes list is derived from imprinted genes lists at the websites http://www.geneimprint.com and http://cancer.otago.ac.nz/IGC/Web/home.html. A list of 512 p53 regulated genes was derived from microarray data of the MDAH041 cell line stably expressing the tetracycline inducible p53 gene. Three preparations of RNA from MDAH041 were harvested at 0, 7, 24 and 72 hours after induction of p53. cRNA preparation and microarray assays were performed as described above. Each of the post p53 induction time points was compared to the 0 time point. Genes that increased or decreased upon expression of p53 were selected using Affymetrix DMT version 5. The p53 regulated gene list is comprised of genes that either increased or decreased at one of the time points following induction of p53, across 65% of the comparisons.

To determine in a particular list if the probability that the number of IFN or p53 is statistically significant, 1 minus cumulative distribution function (cdf) was employed (http://edpsych.ed.sc.edu/seaman/edrm712/questions/onesample.htm) (Draghici et al. 2003).

Results

Previously the gene expression changes during immortalization of the telomerase positive LFS immortal cell line MDAH041 and the role of methylation-dependent gene silencing in that process were analyzed (Kulaeva et al. 2003). To further examine the significance of the role of the IFN pathway and potentially identify other mechanisms commonly abrogated during immortalization, the study was expanded to include three independent LFS cell lines derived from the fibroblasts of a second LFS patient, MDAH087. The three MDAH087 telomerase negative, ALT cell lines (Bischoff et al. 1990; Gollahon et al. 1998), MDAH087-N, MDAH087-1 and MDAH087-10, in addition to the telomerase positive cell line MDAH041, were used in a systematic analysis of the gene expression changes during immortalization and after 5-aza-dC treatment.

p53, p16^{INK4a} and p21^{CIP1/WAF1} Status in the Immortal Cell Lines

To demonstrate that the immortal cell lines MDAH041, MDAH087-N, MDAH087-1 and MDAH087-10 resulted from independent immortalization events and were not replicates of a single cell line, a limited genotyping of key genes known to change during this process was performed. MDAH041 has a p53 germline mutation in exon 5, and MDAH087 has a p53 germline mutation in exon 7. Spontaneous immortalization of MDAH087 occurs in mechanistically distinct fashion among the three immortal variants. Initially, MDAH087 cells have one wild-type and one mutated p53 allele; the mutant p53 allele has a missense mutation (CGG (Arg)→TGG (Trp)) in exon 7, codon 248 (Malkin et al. 1990; Yin et al. 1992). The p53 gene was sequenced in the cell lines used in this study to ensure that precrisis MDAH087 (MDAH087-PC) was heterozygous for p53, and that the three immortal MDAH087 cell lines have lost their wild-type p53. Sequencing confirmed MDAH087-PC was heterozygous for p53. Although MDAH087-N and MDAH087-1 cell lines exhibited loss of heterozygosity (LOH) on chromosome 17 at the p53 gene locus with the loss of the wild copy p53 allele, the MDAH087-10 cell line retained both alleles. Sequencing of cDNA from MDAH087-10 cells revealed that the wild-type p53 allele was altered by a somatically acquired point mutation, resulting in P152G substitution, exon 5. This deleterious mutation has been identified in tumors and is listed in the International Agency for Research on Cancer (IARC) TP53 Mutation Database (http://www.iarc.fr/P53/), mutation identification numbers 1015, 1337, 2976 and 18119. P152G substitution was not found in MDAH087-PC, MDAH087-N or MDAH087-1.

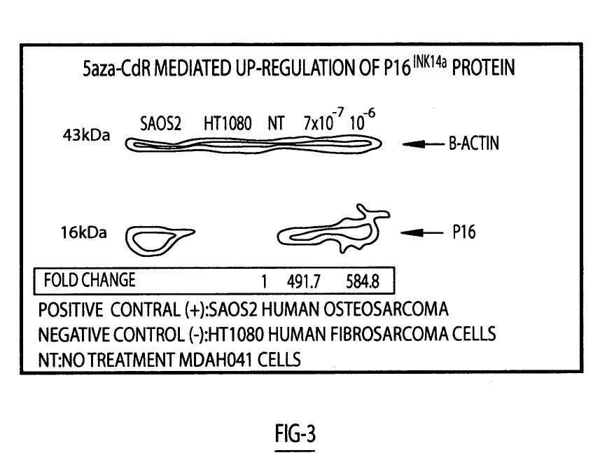

The p53 mutation in MDAH041 causes a premature stop codon, thus in the MDAH041 immortal cells there is no detectable p53 by western blot analysis (FIG. 4). The p53 mutation in MDAH087 has a missense mutation in the DNA-binding domain. The mutant p53 protein found in MDAH087 is readily detected by western blot analysis due to its longer half-life as compared to the wild-type p53 present in normal fibroblasts (FIG. 4).

A second difference among the four immortal LFS cell lines is the protein expression pattern of the cyclin-dependent kinase inhibitors, p16^{INK4a} and p21^{CIP1/WAF1}. In precrisis MDAH041 (MDAH041-PC) cells there is little to no protein expression of p16^{INK4a} or p21^{CIP1/WAF1}. Immortal MDAH041 cells also do not express either p16^{INK4a} or p21^{CIP1/WAF1}, but protein expression of both was induced upon treatment with 5-aza-dC. MDAH087-PC cells express both p16^{INK4a} and p21^{CIP1/WAF1}, but their expression is lost from the immortal MDAH087 cell lines. Treatment of MDAH087-N cells with the DNA methyltransferase inhibitor 5-aza-dC induced protein expression of p16^{INK4a}, but not of p21^{CIP1/WAF1} (Vogt et al. 1998) (FIG. 4). In contrast to MDAH087-N, 5-aza-dC induced expression of p21^{CIP1/WAF1} in MDAH087-1 and MDAH087-10, but not expression of p16^{INK4a} (FIG. 4). These data demonstrate that the four immortal LFS cell lines used in this study contain a different complement of genetic and epigenetic changes, indicating they are independent immortalizations. Analysis of additional genetic or epigenetic events could reveal critical mechanisms of interest to the process of cellular immortalization.

Microarray Profiling of Gene Expression in Immortal LFS Fibroblasts

Total RNA was prepared from each of the LFS cell lines early in their lifespans and as immortal cell lines. Probes were synthesized and hybridized to the Affymetrix HGU95Av2 GeneChip®. The immortal MDAH041 cell line was compared with the MDAH041-PC cell line and the immortal MDAH087 cell lines, MDAH087-N, MDAH087-1 and MDAH087-10, were each individually compared with the MDAH087-PC cell line. All the possible pairings (6 comparisons per cell line) between precrisis versus immortal cell lines were analyzed. In previous studies of the MDAH041 cell line, genes were selected that had at least a 2-fold change in gene expression on the microarrays. The same criteria were used to identify genes that changed and were common to all four immortal LFS cell lines. The Affymetrix Data Mining Tool (DMT) version 5 was used to select genes whose expression increased or decreased, without specification of fold change, in greater than 65% of the chip comparisons for an individual immortal cell line. The number of genes differentially expressed during immortalization, for the each of four LFS immortal cell lines, is shown in Table 7. Less stringent criteria than the 2-fold change criteria was used to identify 897 genes with upregulated expression and 1,120 genes with downregulated expression changes in MDAH041. In the three immortal MDAH087 cell lines, the number of genes with increased or decreased expression ranged from 785 to 1,267. Using this approach there were found 149 upregulated and 187 downregulated genes common to all four immortal LFS cell lines. In general, 98% of the changes in gene expression were greater than 1.5-fold, and 67% were greater than 2-fold. Of the 149 upregulated and 187 downregulated genes there were a statistically significant number of p53-regulated genes (29 upregulated genes, p-value=7.48×10⁻⁹, 23 downregulated genes, p-value=0.0005) (Table 8). In addition there were four known imprinted genes, PHLDA2, CD81, MEG3 and NDN (Table 8), among the 187 downregulated genes, further indicating that methylation-dependent silencing is important to the mechanism of cellular immortalization.

The DNA methyltransferase inhibitor 5-aza-dC induces growth arrest and senescence in LFS immortal fibroblasts (Vogt et al. 1998). Treatment of immortal MDAH041 cells with 5-aza-dC induced significant changes in gene expression (Kulaeva et al. 2003). 5-aza-dC treated immortal LFS cells have a senescence-like morphology and exhibit senescence-associated β-galactosidase activity. To further investigate the role of DNA methylation regulated gene expression during immortalization the study was expanded to include the MDAH087 cell lines (MDAH087-N, MDAH087-1, and MDAH087-10). 5-aza-dC treatment of immortal MDAH087 cell lines resulted in a flat senescence-like morphology and the activation of the senescence-associated β-galactosidase activity.

Total RNA, prepared from 5-aza-dC-treated immortal MDAH041 and MDAH087 cell lines, was used to prepare probes for hybridization to the Affymetrix HGU95Av2 GeneChip®. All the possible pairings (6 comparisons for MDAH041; 9 comparisons for each of the MDAH087 cell lines) of treated and untreated immortal cells were analyzed with Affymetrix DMT version 5. To identify genes that increased or decreased after 5-aza-dC treatment, the same criteria as was used for identifying gene expression changes in immortal cells was used; genes were selected whose expression increased or decreased after 5-aza-dC treatment, without specification of fold change, in greater than 65% of the chip comparisons for an individual immortal cell line. In MDAH041 cells, the number of genes with upregulated and downregulated expression is 877 and 803, respectively (Table 7). This is in comparison to the 190 genes with upregulated expression and the 48 genes with downregulated gene expression in a previous study in which a criterion of 2-fold change in expression was used (Kulaeva et al. 2003). For the immortal MDAH087 cell lines, the number of genes with increased or decreased expression ranged from 408 to 772 (Table 7). When compared the four spontaneously immortal LFS cell lines, 185 upregulated and 46 downregulated genes common to all four immortal LFS cell lines were identified (Table 7). None of the 185 upregulated or the 46 downregulated genes were known imprinted genes. However there were a statistically significant number of p53-regulated genes whose expression was methylation dependent (7 upregulated genes p-value=0.017, 26 downregulated genes p-value=2.46×10⁻⁵) (Table 8).

Genes Downregulated After Immortalization by Gene Methylation

In the study of the MDAH041 cell line (Kulaeva et al. 2003), 85 genes were repressed during immortalization and upregulated after treatment of these cells with 5-aza-dC. This indicated the significance of methylation-dependent gene silencing to the process of immortalization. In this analysis 14 genes, common to all four immortal LFS cell lines, were identified whose expression decreased during immortalization and increased after treatment with 5-aza-dC (Table 7, Genes in sets B and D). These 14 epigenetically regulated genes are statistically significant when compared to the 4 genes that decreased during immortalization and decreased after 5-aza-dC using an exact binomial test, p-value=0.034. Average fold change in expression of these fourteen genes are listed in Table 9. Among the 14 genes, 11 (78%) of them have computational CpG islands in their promoter regions (1000 bp upstream and 500 bp downstream of the transcriptional start site). This suggested that these 11 genes are repressed by promoter hypermethylation during immortalization. To determine the significance of that observation, 16 genes whose expression decreased in all four immortal LFS cell lines were tested for computational CpG islands, but was not affected by treatment with 5-aza-dC, and 60 randomly chosen genes from the HGU95Av2 microarray GeneChip®. Fifteen out of the 16 downregulated methylation insensitive genes (94%) and 49 out of the 60 randomly chosen genes (82%) had computational CpG islands. Focusing the search region to 500 bp upstream and 200 bp downstream of the transcriptional start site reduced only the number of computational CpG islands in the random gene set by 2 (47/60; 78%) and had no effect on the presence of computational CpG islands in the 14 methylation sensitive downregulated genes or the 16 methylation insensitive downregulated genes. The percentage of genes that were identified as having a CpG island in their promoter is approximately the same as the percent that is generally found in the human genome (Antequera 2003). It is evident that the presence of a computational CpG island is not a dependable measure that a gene is actually regulated by promoter methylation.

There were only 2 genes identified that were upregulated during immortalization and decreased after 5-aza-dC treatment in immortal cells, cell division cycle 25B (CDC25B) and LIM domain-binding 2 (LDB2) (Table 7, Genes in sets A and C). This evidence supports the observation (Kulaeva et al. 2003) that methylation-dependent gene silencing is more frequently associated with the spontaneous immortalization of LFS cell lines.

Epigenetic Control of Interferon Regulated Genes in the LFS Cell Lines

In a study, 39 of the 85 genes identified in MDAH041 as epigenetically repressed during immortalization were known to be regulated in the IFN signaling pathway (Kulaeva et al. 2003). The probability of that occurring by chance was 8.31×10⁻⁴⁷, and thus it was concluded that the IFN-pathway genes play a significant mechanistic role in the acquisition of cellular immortalization. This probability was calculated based on having 137 IFN-regulated genes out of 8,628 unique genes on the HGU95Av2 GeneChip®. Based on more recent information there are 1,061 IFN-regulated genes and there are 8,903 unique genes on the HGU95Av2 GeneChip®. Using this updated information it was determined that in MDAH041 cells the number of IFN-regulated genes that are epigenetically regulated during immortalization remained statistically significant (p-value=2×10⁻¹⁵). In the individual MDAH087 immortal cell lines, the number of IFN-regulated genes that decreased during immortalization and increased after 5-aza-dC treatment was also significant (MDAH087-N, p-value=3×10⁻⁶; MDAH087-1, p-value=1.3×10⁻⁷; MDAH087-10 p-value=1.2×10⁻⁶). However, the set of epigenetically silenced IFN genes found in common to all four immortal LFS cell lines was not significant (p-value=0.51). Therefore, it was concluded that although the interferon pathway appears to play a significant role in the cellular immortalization of LFS cell lines, there are differences among the cell lines in the specific genes of this pathway that are dysregulated.

To further evaluate the IFN pathway in MDAH041 the expression of the IFN signaling pathway gene, STAT1α, was determined by western blot analysis (FIG. 4). Consistent with STAT1α transcript expression (Table 10), protein levels of STAT1α decreased in immortal MDAH041 cells and increased in response to treatment of immortal MDAH041 cell with 5-aza-dC. Changes in STAT1α protein (FIG. 4) and mRNA (Table 10) expression were also analyzed in MDAH087 cells. As was seen in MDAH041, STAT1α gene expression decreased during immortalization and increased after 5-aza-dC treatment in MDAH087 cells. Interestingly, in contrast to Q-RT-PCR and microarray data, there was no difference in protein expression levels of STAT1α between MDAH087-PC and the immortal MDAH087 cell lines, nor was there found a difference in levels between untreated and 5-aza-dC treated samples (FIG. 4). Furthermore after stimulation of LFS cells with either VSV viral infection (Balachandran et al. 2000) or the double-strand RNA analog poly (I:C), which mimics viral infection, it was found that the cell lines were differentially sensitive to these treatments. While poly (I:C) treatment induced STAT1α and IFNβ gene expression in MDAH041-PC, MDAH087-PC, immortal MDAH041 and MDAH087-N, there was no induction of STAT1α and IFN β in either MDAH087-1 or MDAH087-10. Because different sets of IFN regulated genes were dysregulated in each of the four immortal LFS cell lines, it was concluded that while the specific mechanism of inactivation of the IFN pathway varies from cell line to cell line, abrogation of this significant pathway is necessary, albeit not sufficient, for immortalization to occur.

Confirmation of Microarray Gene Expression Data by Q-RT-PCR Analysis

Q-RT-PCR was used to confirm the gene expression changes observed in the microarray data (Table 10). The expression of nine representative genes was examined, CREG, IGFBPrP1, CLTB, KIAA1750, OPTN, HSPA2, TNFAIP2, ALDH1A3 and SERPINB2, among the fourteen genes that were identified as decreased during immortalization and increased after 5-aza-dC treatment in all four immortal LFS cell lines. Three of the genes, CLTB, HSPA2 and OPTN, in the list of fourteen genes that were epigenetically regulated during immortalization, had multiple probes on the Affymetrix HGU95Av2 GeneChip®. Although all of the replicate probes on the chip for these genes were not identified as decreased during immortalization and increased after 5-aza-dC treatment in all four immortal LFS cell lines, Q-RT-PCR did in fact confirm that these genes were in fact epigenetically regulated during immortalization. In addition, as IGF-binding proteins (IGFBP) are known to be involved in cellular immortalization, two additional IGFBP genes, IGFBP3 and IGFBP4, were also analyzed by Q-RT-PCR; both of these genes decreased during immortalization among all four immortal LFS cell lines. Overall 96% (Table 10, 92 out of 96 comparisons) of the changes in gene expression observed in the microarrays were confirmed by Q-RT-PCR. Microarray and Q-RT-PCR fold-changes were considered in accordance when either both had significant fold changes in the same direction, or neither had a significant fold-change. The cutoff for significant fold-change for microarray was ±1.3-fold, and for Q-RT-PCR was ±1.8-fold. In 3 of the 4 instances when there was a discrepancy between microarray and Q-RT-PCR gene expression changes, the microarray fold-change was low, less than 1.1-fold, or the Q-RT-PCR fold-change was low, less than 0.61-fold. In one instance, IGFBP3 gene expression changes in MDAH087-10 after 5-aza-dC treatment, the microarray fold-change was significant (−2.66), but the Q-RT-PCR fold-change was not significant (−1.4), thus microarray and Q-RT-PCR fold-changes in concordance were not considered. These few discrepancies between microarray and Q-RT-PCR fold-changes were isolated instances that only occurred in 2 of the 12 genes analyzed, HSPA2 and IGFBP3. Furthermore, for HSPA2 there was only one such discrepancy. Thus microarray is a reliable method to measure expression changes.

Confirmation of Gene Expression Data by Western Blot Analysis

Western blot analysis was employed to determine if protein expression data correlated with changes in gene expression (FIG. 4). Consistent with microarray and Q-RT-PCR data, IGFBP3 protein levels decreased during immortalization in MDAH041, MDAH087-N, MDAH087-1 and MDAH087-10 cell lines, but only increased in response to 5-aza-dC in one LFS cell line, MDAH041. There was a decrease in expression of IGFBP4 in all four immortal LFS cell lines. IGFBP4 runs as a doublet, the higher band likely represents the glycosylated form of this protein (Carr et al. 1994). Interestingly, the glycoslyated form of IGFBP4 is more prevalent in the immortal cell lines than in the precrisis cell lines (FIG. 4). After 5-aza-dC treatment there was a slight induction of IGFBP4 in MDAH041, MDAH087-N and MDAH087-1, but no induction in MDAH087-10. This is in contrast to gene expression data (Table 10), where IGFBP4 was not induced in MDAH087-1 cells and was induced in MDAH087-10 cell following 5-aza-dC treatment. Also inconsistent with microarray and Q-RT-PCR data, there was little to no induction of either IGFBP4 or IGFBPrP1 protein (FIG. 4) in MDAH041 following treatment with 5-aza-dC. Q-RT-PCR confirmed the microarray data by an independent method demonstrating that the microarray gene expression data is accurate. The rare instances when there was a difference between Q-RT-PCR and microarray data with western blot data suggests that posttranslational mechanisms, not reflected in the gene expression data, control the protein expression from these genes during immortalization.

Global Analysis of Gene Expression by Hierarchical Clustering

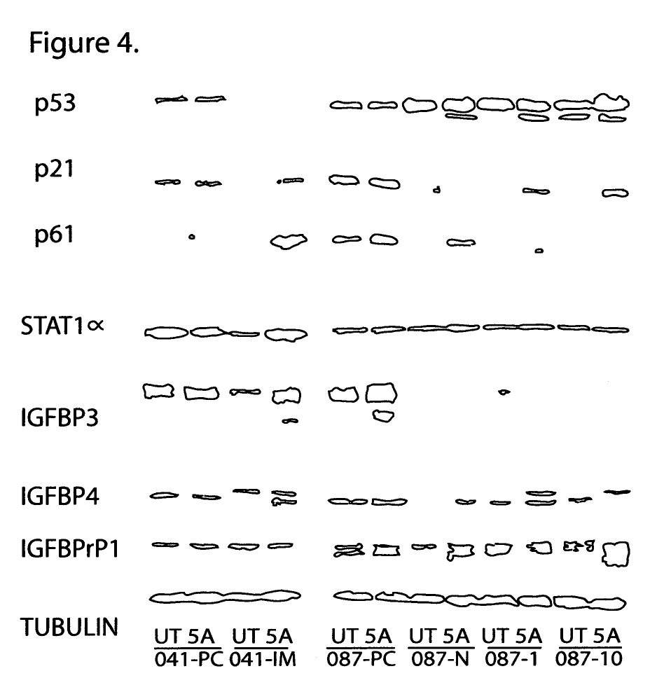

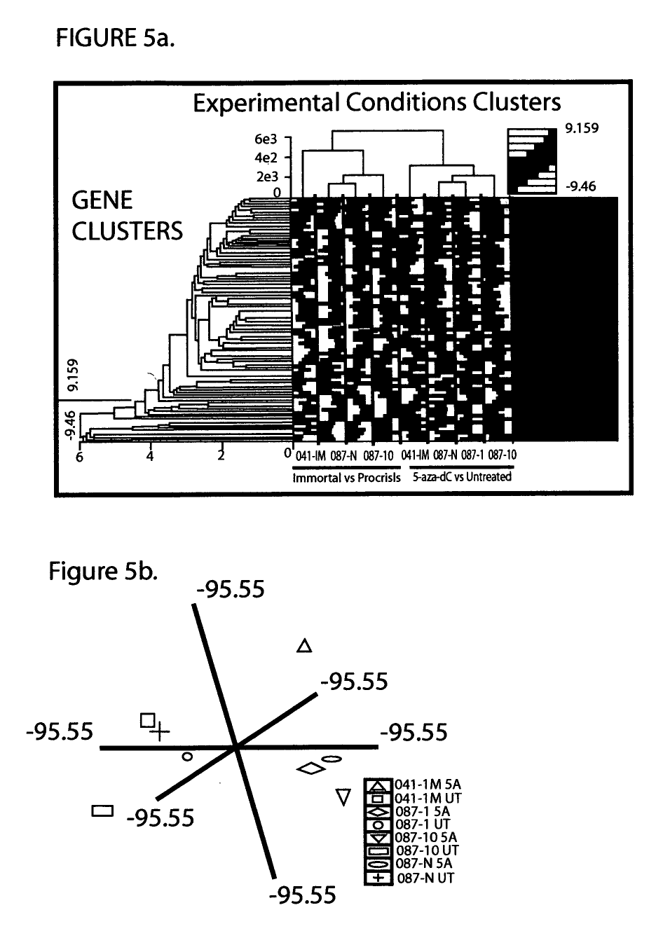

MDAH041, MDHAO87-N, MDAH087-1 and MDAH087-10 are independent immortalizations of human skin fibroblasts with apparent similarities in the mechanisms by which they became immortal. To globally assess these mechanistic similarities the entire gene expression data set was analyzed using hierarchical clustering (FIG. 5a). The immortal versus precrisis cells expression datasets cluster distinctively from the 5-aza-dC versus untreated immortal cell expression datasets. The expression patterns revealed by the hierarchical cluster map show that the two processes, immortalization and demethylation, have reciprocal changes in gene regulation. These data support the observation that the treatment of immortal MDAH041 and MDAH087 cell lines with 5-aza-dC can reverse the immortal phenotype, and cause the cells to senescence (Kulaeva et al. 2003; Vogt et al. 1998).

To determine if the profiles of any two pairs of cell lines more closely resembled one another than to the other pairs of cell lines, the number of genes common between all possible pairings of the immortal cell lines was examined. By this analysis there were no pairs of immortal cell lines that were more alike. However, upon examination of the expression data using hierarchical clustering it was found that the three immortal MDAH087 cell lines are more similar to one another than MDAH041 is with any one of them. It was not surprising that MDAH041 and MDAH087 immortal cell lines have differences in genes expression patterns, as these cell lines seem to be in different immortalization complementation groups (Gollahon et al. 1998), and MDAH041 is telomerase positive while MDAH087 telomeres stabilize via ALT. Among the three MDAH087 cell lines, MDAH087-N and MDAH087-10 were more closely related to one another than the other possible pairings of the MDAH087 cell lines. MDAH041 clustered separately from the three MDAH087 cell lines. While immortal MDAH087-N and MDAH087-10 were the closest of the possible immortal cell pairs, after 5-aza-dC treatment MDAH087-N had a gene expression pattern that was more similar to MDAH087-1 than to MDAH087-1 0.

Upon examination of the 5-aza-dC cluster diagram, only a small subset of genes was regulated by 5-aza-dC in common to all four immortal LFS cell lines. Yet, within any immortal cell line, there are hundreds of genes whose expression increases following treatment with 5-aza-dC. This suggests that among the four immortal LFS cells lines, while there are similar pathways involved in bypassing senescence, and in induction of senescence with 5-aza-dC, different sets of genes are regulated in each of the immortal LFS cell lines. Despite individual differences in the genes dysregulated during immortalization among the four immortal LFS cell lines, methylation-dependent gene silencing is necessary for LFS cells to bypass senescence and become immortal.

Multidimensional scaling, like hierarchical clustering analysis is based on evaluating the similarity distance of the expression data and is used to reveal the relationship between the samples; however in multidimensional scaling the samples are plotted in a three-dimensional space (FIG. 5b) and thus it provides another approach to visualizing the data. The three-dimensional models allow a more straightforward visualization of the similarities among the sample pairs than hierarchical clustering diagrams. The distance of each sample pair in the three-dimensional space represents their Euclidean distance. The four colored balls, which represent each of the immortal cell lines, were relatively far from the balls that represented the 5-aza-dC treated immortal cell lines (FIG. 5b). Among the four immortal LFS cell lines, the MDAH041 cell line is set apart from the three MDAH087 cell lines, reflecting that MDAH041 has a different set of genes that are differentially expressed during immortalization than the MDAH087 immortal cells lines. Of the MDAH087 immortal cell lines, MDAH087-N and MDAH087-10 were closer to each other in the immortalization comparisons; however in the 5-aza-dC comparisons, MDAH087-N and MDAH087-1 expression patterns were closer to each other. Overall the relationship among the four immortal LFS cell lines determined using multidimensional clustering analysis agrees with the results from the hierarchical clustering analysis.

Identification of Pathways Associated with Cellular Immortalization

To identify mechanisms, in addition to the IFN-regulated pathway, critical to cellular immortalization, the biological, cellular and molecular characteristics of the genes differentially expressed during immortalization and after 5-aza-dC treatment (Table 7) were studied using the Gene Ontology (GO) software program, GoMiner. GoMiner links genes with GO categories, allowing one to identify the biological process, cellular component and molecular function associated with the genes in the lists (Zeeberg et al. 2003). The p-value for each GO category was calculated as an evaluation of the significance of the number of gene changes associated with each GO category. The categories that had five or more genes that changed, or had an uncorrected p-value <0.01 are reported in Table 14. Subcategories (Table 14, categories in bold), of the three primary GO categories biological processes, cellular components and molecular function, were graphed, using the −log₁₀(p-value) (FIG. 6) to facilitate evaluation of which functional categories were significant during immortalization and or 5-aza-dC treatment. Sublayers of the significant GO categories were then explored in finer detail to determine more specifically which GO functional categories accounted for the changes. To avoid misinterpretations resulting from failure to correct for multiple comparisons in GoMiner, the p-values were recalculated using False Discovery Rate (FDR) (FIG. 6, denoted by asterisks) for each GO category. Possibly due to the small number (<200) of dysregulated genes in the lists, there were only a few GO categories that achieved significance after correction by FDR.

In the primary GO category biological processes, cell adhesion (GO:0007155) and cell motility (GO:0006928) are among the subcategories (FIG. 6a; Table 14a) that had a significant number of genes that were downregulated during immortalization. Among the categories with a significant number of genes upregulated during immortalization were cell proliferation (GO:0008283), metabolism (GO:0008152), and cell organization and biogenesis (GO:0016043). After correction by FDR, only cell proliferation (GO:0008283) remained significant. Within the cell proliferation (GO:0008283) category most of the changes occurred in cell cycle (GO:0007049), cytokinesis (GO:0000910) and regulation of cell proliferation (GO:0042172), but only cell cycle (GO:0007049) remained significant after correction by FDR. In these categories, MYC, E2F transcription factor 4 (E2F4), cyclin-dependent kinase inhibitor 1A (CDKN1A) and cyclin-dependent kinase inhibitor 3 (CDKN3) were among the genes identified as dysregulated during immortalization. Identification of these GO categories and genes, supports the observation that dysregulation of the cell cycle, through the retinoblastoma signaling pathway, and dysregulation of cyclin-dependent kinase inhibitors, are required for immortalization of fibroblasts (Kiyono et al. 1998; Tsutsui et al. 2002). Through classification of genes in GO categories known genes in the cell cycle pathway that are involved in immortalization were identified, thus this data indicated that one can identify other pathways that are involved in immortalization using this approach.

After treatment with 5-aza-dC, the categories in the primary GO category biological process with a significant number of genes with changes in gene expression included cell death (GO:0008219), cell proliferation (GO:0008283), response to stress (GO:0006950), and cell organization and biogenesis (GO:0016043). Genes in these categories include cell division cycle 34 (CDC34), cyclin-dependent kinase inhibitor 2C (CDNK2C) and fibroblast growth factor 2 (FGF2). During immortalization, a significant number of genes in the cell proliferation category increased in expression, and after 5-aza-dC treatment a significant number of genes in the cell proliferation category decreased in expression. Interestingly, in the cell proliferation category (GO:0008283), among the 35 genes (Table 15) whose expression was upregulated during immortalization and the 11 genes whose expression was downregulated following demethylation, only one gene was found in both gene sets, CDC25B.

Of the GO categories identified for the genes differentially expressed after 5-aza-dC treatment, the only GO category that remained significant after correction by FDR was response to wounding (GO:009611), a subcategory of response to extracellular stimulus (GO:0009991). This is consistent with the finding that the IFN pathway is important in immortalization (Kulaeva et al. 2003), as several of the genes identified in the wounding category (GO:009611) are interferon and/or cytokine regulated genes (Table 16).

In the primary GO category cellular component (FIG. 6B), genes downregulated during immortalization were primarily in the GO categories extracellular (GO:0005576), cytoplasm (GO:0005737) and a subcategory of cytoplasm, cytoskeleton (GO:0005856), while the genes with upregulated expression were in the GO categories chromosome (GO:0005694) and nucleus (GO:0005634), and subcategories of cytoplasm (GO:0005737) and mitochondrion (GO:0005739). However only the GO categories nucleus (GO:0005634) and cytoskeleton (GO:0005856) remained significant after correction by FDR. The cytoskeleton (GO:0005856) category is consistent with the morphological changes that occur as cells senesce; typically, as cells senesce they become very large and flat. Thus, one would predict that changes in cytoskeletal genes would contribute to the processes of immortalization and senescence. The genes that were upregulated after treatment with 5-aza-dC were in GO categories extracellular (GO:0005576), nucleus (GO:0005634), and chromosome (GO:0005694). Only chromosome (GO:0005694) remained significant after correction by FDR. There were no subcategories of cellular component with a significant number of genes that decreased after 5-aza-dC treatment.

In the primary GO category molecular function (FIG. 6C), genes that were upregulated during immortalization were in the subcategory catalytic activity (GO:0003824) and those downregulated during immortalization were in the subcategories cell adhesion molecule activity (GO:0005194) and structural molecule activity (GO:0005198). Although none of these categories retained significance after correction by FDR, the identification of structural molecule activity (GO:0005198) in molecular function is consistent with cytoskeleton genes (GO:0005856) being identified as significant in the cellular component. Sixteen of the 24 genes from structural molecular activity genes (GO:0005198), and 1 of the 9 genes in the cell adhesion molecular activity (GO:0005194), overlap with the genes in the cytoskeletal category (GO:0005856) (Table 17). Additionally the cytoskeletal protein, gelsolin, is epigenetically regulated in MDAH041 immortal cells. There were two GO categories examined that were both significantly downregulated during immortalization and upregulated during demethylation: a subcategory of biological process, regulation of cell proliferation (GO:0042127, p-value IM_Down <0.02; p-value 5A_Up <0.05) and a subcategory of cellular component, extracellular (GO:0005576, p-value IM_Down <0.005; p-value 5A_Up <0.01). The genes downregulated during immortalization did not overlap with the genes upregulated during demethylation in the regulation of cell proliferation category (GO:0008283), except for the gene IGFBPrP1. This finding indicates that the arrest of cell proliferation (GO:0008283) associated with 5-aza-dC-treatment results in changes in a different set of genes from those altered to permit cells to maintain their proliferative capacity during immortalization.

Regional Control of Gene Expression

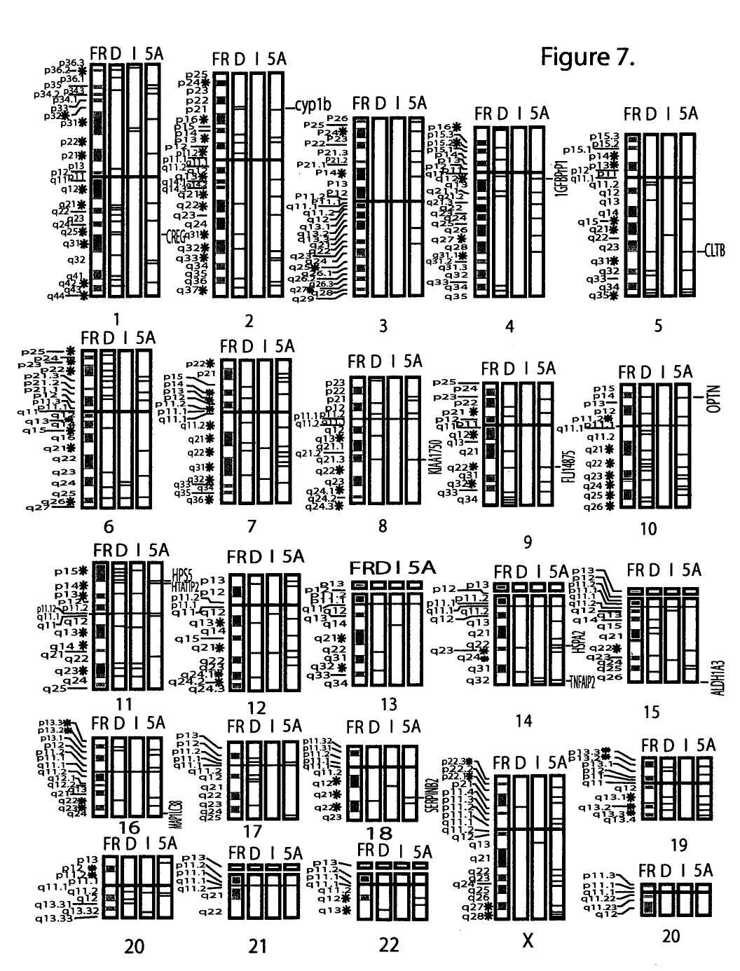

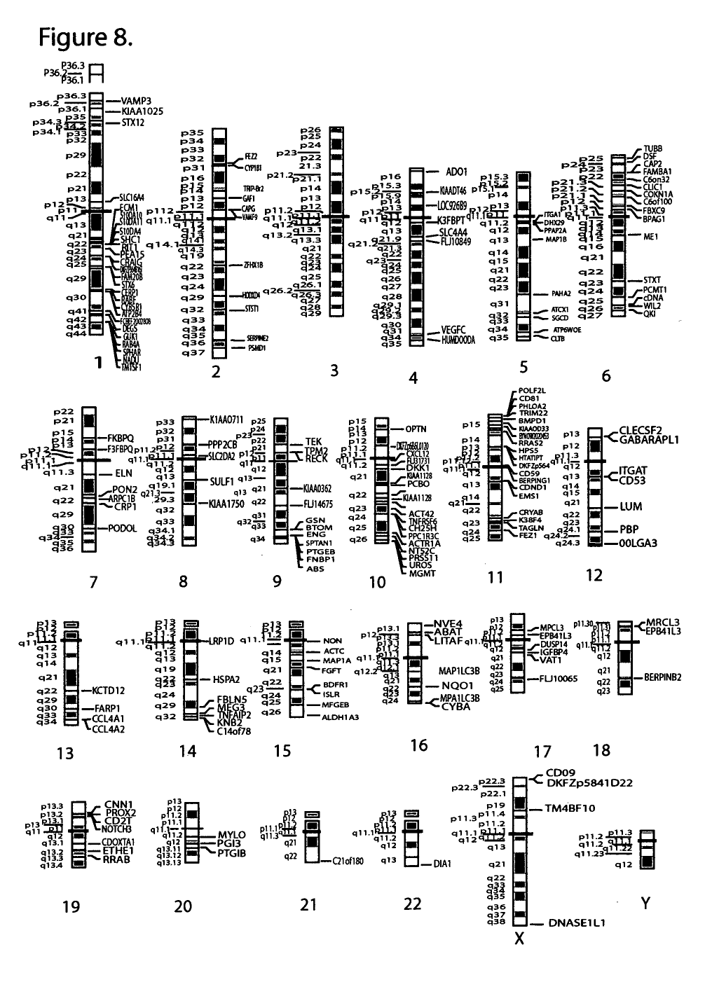

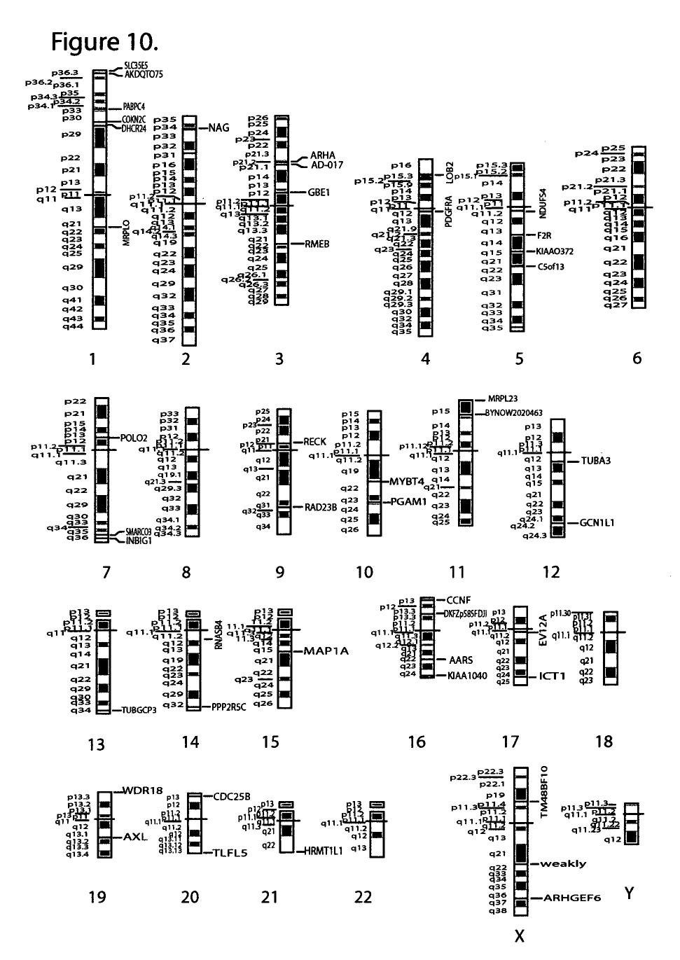

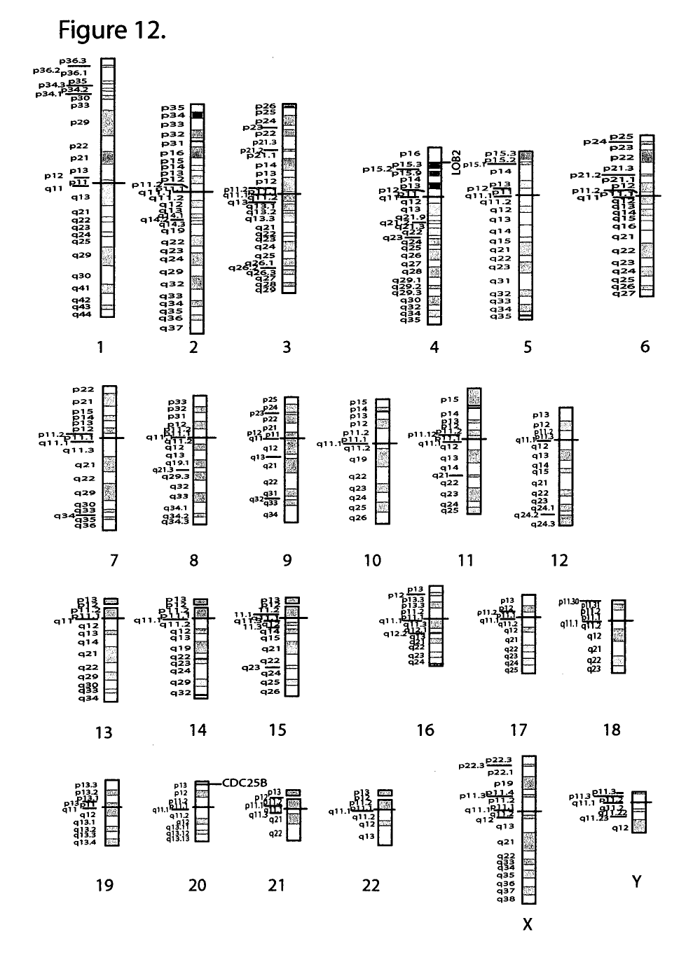

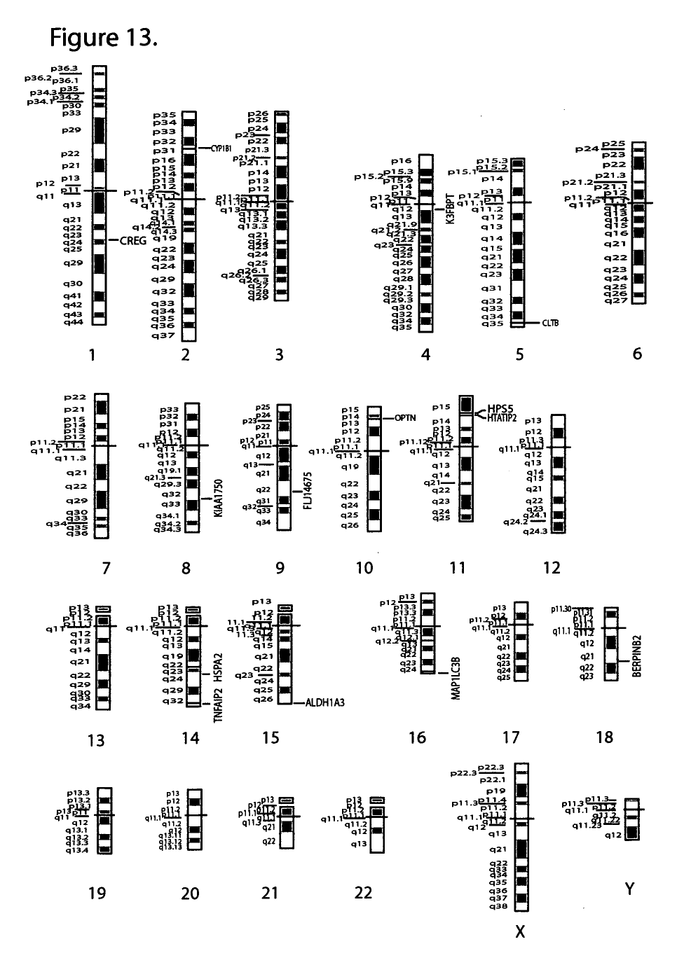

To determine whether epigenetic regulation of gene expression is controlled in a regional fashion on certain chromosomes during immortalization, chromosome ideograms were annotated to indicate areas of altered gene regulation (FIGS. 7-13). Genes with an increase in expression during immortalization were found on chromosome 3p, 12, 14q, 17q21, 17q23, 19p13.3, 19q13, 20 and 22 (FIG. 8). Of interest, there are no genes on chromosome 3 that decreased in expression during immortalization in common to all four immortal LFS cell lines. However, there are genes on chromosome 3 that decrease in expression during immortalization that are in common to the three MDAH087 immortal cell lines. This is intriguing because introduction of normal chromosome 3 into a renal cell carcinoma cell line and an ovarian carcinoma cell line induces senescence (Horikawa et al. 1998; Rimessi et al. 1994; Tanaka et al. 1998); induction of senescence was attributed to a gene on chromosome 3p14.2-p21.1 decreasing telomerase activity (Horikawa et al. 1998; Tanaka et al. 1998). The reason that there may not be any genes decreased on chromosome 3 that are in common to MDAH041 and the three MDAH087 immortal cell lines may be a reflection of how the telomeres are stabilized in these cell lines during immortalization, MDAH041 by increased telomerase activity and MDAH087 by ALT. Thus, in order for MDAH041 to bypass senescence and become immortal, genes on chromosome 3 that naturally inhibit telomerase activity are selected against. As MDAH087 immortal cell lines stabilize telomeres by ALT, there is no selective pressure against genes that inhibit telomerase activity. Therefore one or more of the chromosome 3 genes that are specifically downregulated in MDAH041 cells may be a critical negative regulator of telomerase that is lost when these cells become immortal. When the four immortal LFS cell lines were individually analyzed, in each of the cell lines, 19q13 is a region that had a large number of genes with increased expression. In all three immortal MDAH087 cell lines, both chromosomes 17 and 22 had a cluster of genes that increased during immortalization. In MDAH041, chromosome region 20q had a cluster of genes that increased during immortalization, which was not observed in the MDAH087 immortal cell lines.

Decreased gene expression was found during immortalization in all four immortal LFS cell lines on multiple chromosomes including 1q21, 1q32, 1q41, 6q, 9q34, 10, 11p15, 11q23, 13q, 14q32 and 15 (FIG. 9). In the analysis of the individual cell lines, in each of the cell lines, 10q is a region with a group of genes whose expression decreases during immortalization. In MDAH041 and MDAH087-N there is a group of genes with decreased expression during immortalization at 9q22 and 9q32. In MDAH041, MDAH087-N and MDAH087-1 there are clusters of genes at 6q and 11q that decrease during immortalization. In all cell lines except MDAH087-1 there are clusters of genes at 9q34 and 14q32 that have a decrease in expression during immortalization. Also of interest are chromosomes 4, 6 and 13. There is only one gene on chromosome 4 with increased expression during immortalization, but there are three genes on chromosome 4p and five genes on chromosome 4q with decreased expression in all four immortal LFS cell lines during immortalization. On chromosome 6p there are both genes with increased expression and with decreased expression during immortalization, but on chromosome 6q there are only genes with a decrease in expression during immortalization. Common to all four immortal LFS cell lines there are four genes located on chromosome 13 with a decrease in expression, but no genes on this chromosome with an increase in expression after immortalization. Of note, these genes are located in the region of chromosome 13q22 to 13q32 and are not located near RB, which is at chromosome 13q14.2. There is LOH along the q arm of chromosome 13, including the region where RB is located, in MDAH041, MDAH087-1 and MDAH087-10, but RB protein expression is unaffected. Thus, the decrease in expression of the four genes is a consequence of combination of mechanisms, such as LOH in combination with methylation or gene mutations. Genes with expression that decreased during immortalization and increased after 5-aza-dC treatment, in common to all four immortal LFS cell lines, cluster on chromosome 4q12-q27, 6p22, 6p21.3, 7,14, 19 and X (FIGS. 9 and 11).

Discussion

Tumors evolve from normal cells due to a series of genetic and epigenetic changes that result in phenotypic alterations found in cancer cells. One of the earliest identifiable phenotypes is that of escaping cellular senescence, immortalization, which provides the proliferative capacity necessary for a tumor to develop. A number of genetic factors have been shown to play a role in the acquisition of the immortal phenotype including changes in tumor suppressor genes such as p53 and p16, oncogenes such as c-myc, and the upregulation of the enzyme telomerase. Telomerase provides protection of telomeres whose erosion results in a reduction in the cells proliferative capacity. Such genetic changes will provide molecular targets for intervention at the earliest stages of cancer development. LFS cells spontaneously immortalize in cell culture without the aid of chemical mutagens or transforming viruses, and as such provide a useful model system to study cellular immortalization. The goal was to confirm the role of IFN genes in the process of immortalization using three independent immortal cell lines derived from a second LFS cell line, MDAH087. In the analysis there were identified several pathways with changes in gene expression, including the interferon signaling pathway, the cell cycle pathway, and genes for proteins in the cytoskeleton, that were differentially expressed after the immortalization in LFS cells. Fourteen genes were consistently epigenetically regulated during immortalization in all of the immortal cell lines studied.

Hierarchical clustering and multidimensional analysis was used to determine the relationships between the four immortal LFS cell lines, and to identify genes that were similarly regulated in the four immortal LFS cell lines. Both approaches indicated that the three immortal MDAH087-derived cell lines, although independently immortalized, were more closely related to one another than MDAH041 was to any of these cell lines. As expected, the gene expression patterns were closely related, but not identical among the three MDAH087 immortal cell lines.

Data from another study suggested that genes with a similar expression pattern may be functionally related (Allocco et al. 2004). Although the gene expression patterns were not identical in the four immortal LFS cell lines, the Gene Ontology Pathways in which the genes are classified were similar. Thus certain pathways, but not necessarily particular genes, must be abrogated or enhanced in order for a cell to become immortal. The mechanisms by which a particular pathway is disrupted varied among cell lines. A significant number of the epigenetically regulated genes, in each of the four immortal LFS cell lines, are in the IFN pathway. The involvement of the IFN pathway in cellular senescence and tumorigenesis is supported by the fact that a number of IFN induced proteins have tumor suppression activity when overexpressed in tumor cells. These proteins include double stranded RNA activated protein kinase (PKR), activated RNaseL, and the 200 gene family (Pitha 2000). Recent studies examining the promoter methylation in bladder cancer cells and colon cells also showed the activation of IFN signaling pathways after the treatment of cancer cells with 5-aza-dC implying that their promoters are silenced by DNA methylation (Karpf et al. 1999; Liang et al. 2002). The data were the first to demonstrate that IFN signaling pathways were silenced by methylation in an early step of cancer development, immortalization (Kulaeva et al. 2003). These results support the hypothesis that genes in the IFN signaling pathways may act as growth suppressors in the progression of cells to immortalization. Recently, the interferon inducible gene, IFI 16, was shown to contribute to senescence in prostate epithelial and fibroblast cells (Xin et al. 2003; Xin et al. 2004).

By categorizing the genes in gene ontology categories, it was determined that genes coding for regulatory proteins of the cell cycle and/or structural proteins of the cytoskeleton were differentially expressed during immortalization. Identification of the cell cycle as a significant pathway is consistent with studies that found that the cell cycle is dysregulated during immortalization (Vogt et al. 1998; Yin et al. 1992). In the cell cycle pathway, in addition to the well-known cell cycle genes, RB and p16^{INK4}, that are involved in cellular immortalization, other cell cycle regulators were found among the fourteen genes epigenetically regulated in all four immortal LFS cell lines. These include CREG and SERPINB2, which are mechanistically involved with the RB protein, contribute to cellular immortalization and CREG which causes a delay in G1/S transition when overexpressed in NTERA-2 cells (Di Bacco and Gill 2003).

Two of the fourteen epigenetically regulated genes, MAP1LC3B and HPS5 are associated with the cytoskeleton. The cytoskeletal protein CRP1, which happens to be regulated by IFN, decreases during immortalization in all four immortal LFS cell lines, further supporting the involvement of the IFN pathway and cytoskeletal proteins in immortalization.

The fourteen epigenetically regulated genes that are common to all four immortal LFS cell lines do not have a significantly higher percent of genes with CpG islands when compared to another set of genes that were similarly downregulated in immortal cells but not regulated by 5-aza-dC. In addition, the size of the CpG island(s) was not found within the epigenetically regulated genes correlated with their being epigenetically regulated. Of these fourteen genes, there are twelve genes that have a known function, nine of which, IGFBPrP1, ALDH1A3, SERPINB2, also known as PAI-2, CREG, TNFAIP2, HTATIP2, CYP1B1, HPS5 and MAP1LC3B, have been associated with tumorigenesis, senescence, CpG methylation or the cytoskeleton, and based on microarray analysis and literature (Antalis et al. 1998; Der et al. 1998); D. Leaman, personal communication), three are regulated by IFN, ALDH1A3, OPTN and SERPINB2.

Four of the fourteen epigenetically regulated genes may regulate the cell cycle by being functionally associated with p53 or RB. IGFBPrP1, and ALDH1A3 are regulated by p53, and as previously discussed SERPINB2 and CREG associate in cells with RB. In addition, CDC25B, which is increased in expression after immortalization and decreased in expression after 5-aza-dC treatment, is also regulated by p53. Consistent with the findings that SERPINB2 decreases during immortalization and increases after 5-aza-dC treatment, it is overexpressed in skin cells when they senesce (West et al. 1996) and SERPINB2 decreases 25-fold after the transformation of RHEK-1 cells (Yang et al. 1999). In addition, using the CGAP Virtual Northern blot of EST libraries, SERPINB2 is expressed in normal skin but not in cancerous skin. In keeping with the hypothesis that the IFN pathway plays a key role in cellular immortalization, SERPINB2 was found to protect cells from alpha virus infection through the induction of IFN-stimulated gene factor 3 (ISGF3) and through the induction of a low-level interferon-alpha/beta production (Antalis et al. 1998). Interestingly, like CYP1B1, another of the fourteen epigenetically regulated genes, SERPINB2 is regulated by 2,3,7,8-tetrachlorodibenzo-p-dioxin (TCDD) (Jana et al. 2000) through ligand-mediated activation of for the aryl-hydrocarbon receptor (AhR) (Bock 1994). This suggests that AhR and the genes regulated by it may play a role in regulating senescence. This hypothesis is corroborated by Ray and Swanson who found AhR protein levels increase during senescence of keratinocytes (Ray and Swanson 2004). In addition, TCDD causes transcriptional silencing in part through promoter methylation and under these conditions AhR is involved in inhibiting senescence of primary human epidermal keratinocytes (Ray and Swanson 2004).

HTATIP2 and TNFAIP2 are among the fourteen genes epigenetically regulated in all four immortal cell lines. HTATIP2 is a putative tumor suppressor gene that promotes apoptosis and inhibits angiogenesis (Ito et al. 2003). The loss of HTATIP2 increases fibroblast transformation and ectopic expression of HTATIP2 leads to growth suppression. Furthermore HTATIP2-null mice are more susceptible to tumor development, including hepatocellular carcinomas (Ito et al. 2003). TNFAIP2 is cytokine/retinoic acid-inducible (Rusiniak et al. 2000). As retinoids are able to induce senescence-like growth arrest in tumor cells (Lotan and Nicolson 1977; Ma et al. 2003; Roninson and Dokmanovic 2003; Rusiniak et al. 2000), the findings may indicate that induction of senescence by retinoids is at least partially through the induction of TNFAIP2.

Of the fourteen genes decreased during immortalization and increased after 5-aza-dC, of particular interest was IGFBPrP1. IGFBPrP1 is a member of the insulin-like growth factor-binding protein (IGFBP) superfamily consisting of six, IGFBPs and nine, IGFBP-related proteins (IGFBPrP). In addition to IGFBPrP1, other members of the IGFBP family of genes, including IGFBP3, IGFBP4 and IGFBPrP5 are silenced in the four immortal LFS cell lines. IGFBP3, IGFPB4, IGFBPrP1 and IGFBPrP5 all have CpG islands in their promoters and are potentially silenced during immortalization by methylation of these CpG islands. The IGFBPs and IGFBPrPs are involved in cell proliferation, differentiation, and apoptosis. IGFBPs bind to insulin-like growth factors (IGF-I and IGF-2), and function as their carrier, prolong their half-life, modulate their availability and prevent them from binding IGF-I and IGF-11 receptors (IGF-IR and IGF-IIR) (Hwa et al. 1999; Rajaram et al. 1997). IGFs are able to protect cells from apoptosis, act as mitogens and are necessary for the establishment and maintenance of the transformed phenotype (Benini et al. 2001). A mutation in codon 248 of p53, such as that found in all the MDAH087 cell lines, causes stimulation of the IGF-I-R (Girnita et al. 2000; Werner et al. 1996). In the microarray analysis IGF-I-R increases in all four immortal LFS cell lines, possibly a result of the loss of wild-type p53. The loss of IGFBP3 and IGFBPrP1 may be a consequence of the increase in lifespan due to the loss of p53, which also leads to genomic instability and immortalization.

There are many reports of poor prognosis and increased risk of cancer when IGFBPs and IGFPBrPs are dysregulated. Breast cancer patients with low levels of IGFBPrP1 had a poor prognosis (Landberg et al. 2001). There is an inverse correlation of IGFBP3 with risk and prognosis of prostate, breast, lung, and colorectal cancer (Monzavi and Cohen 2002; Oh et al. 1995; Yu and Rohan 2000). Furthermore it has been shown to inhibit cell proliferation in breast and prostate cancer cells (Hwa et al. 1999; Oh et al. 1993; Oh et al. 1995; Rajaram et al. 1997). In one study 75% of the human hepatocellular carcinomas analyzed had a reduction in IGFBP3 expression and 33% of the hepatocellular carcinomas contained hypermethylation in the IGFBP3 promoter (Hanafusa et al. 2002). Treating hypermethylated hepatocellular carcinoma cell lines with 5-aza-dC re-established the expression of IGFBP3. Thus IGFBP3 may be an epigenetically regulated gene capable of inducing cellular senescence. Increasing levels of IGFBP3 and/or the other IGFBPs may decrease the levels of free IGFs leading to decreased binding of IGF to IGF-IR and IGF-IIR, thereby inhibiting cell growth and proliferation, and/or promoting apoptosis. Because the lifespan of these cells is insufficient to survive at a distant site, it is less likely that they will become immortal and acquire the ability to metastasize. Schwarze et. al. used cDNA microarrays to identify genes in human prostate epithelial cells upregulated in senescence and repressed during immortalization, and found that IGFBP3 was decreased in immortal cells and upregulated in senescent cells (Schwarze et al. 2002). Also consistent with the gene expression analysis of immortal LFS cells, IGFBP3 increases during senescence of human oral keratinocytes (Kang et al. 2003). Similarly, IGFBPrP1 was found to be overexpressed during senescence of human mammary epithelial cells and human prostate epithelial cells (Lopez-Bermejo et al. 2000; Swisshelm et al. 1995). Expression of IGFBPrP1 in MCF-7 breast cancer cells induces a senescence-like state (Wilson et al. 2002). Methylation of IGFBPrP1 corresponds to a decrease in its expression during hepatocarcinogenesis (Komatsu et al. 2000). To test the hypothesis that a decrease in IGFBP genes permits immortalization and subsequently can lead to tumorigenesis, the CGAP Virtual Northern blot of EST libraries were queried and found that IGFBP is expressed in normal liver tissue but not in cancerous liver tissue. Thus the epigenetic regulation of IGFBP3 and IGFBPrP1 is consistent with the findings supporting the involvement of IGFBP genes in senescence.

HSPA2, (HSP70 isoform 2) one of the fourteen epigenetically regulated genes, is hypermethylated in breast cancer, but can be reactivated upon treatment with a demethylating agent (Shi et al. 2002). Using the CGAP Virtual Northern blot of EST libraries, it was found that HSP70-2 is expressed in normal testicular tissue but not in testicular cancer tissue. Interestingly, HSP70-2 disruption in the mouse genome results in male meiosis defects and infertility (Dix et al. 1996).

By microarray and Q-RT-PCR analysis, CYP1B1 was found to be epigenetically regulated in all four immortal LFS cell lines. Consistent with the finding, another lab found that CYP1B1 expression is enhanced during senescence human oral keratinocytes (Kang et al. 2003). Contradictory to these results, CYP1B1 was found to decrease with differentiation of mouse embryo fibroblasts (MEFs), and increase in MEFs that escaped senescence (Alexander et al. 1997). However, it was concluded that while the CYP1B1 mRNA may be altered in expression during immortalization, its function in this process is unlikely to be significant.

There were two genes, CDC25B and LDB2, which increased during immortalization and decreased with 5-aza-dC treatment. Consistent with the findings, CDC25B message is overexpressed in human tumors, including pancreatic, prostate head and neck, and in some cancer cell lines (Gasparotto et al. 1997; Guo et al. 2004; Ngan et al. 2003; Ullmannova et al. 2003). In agreement with the finding that CDC25B decreases after induction of senescence with 5-aza-dC, treatment of cells with another differentiation agent, butyric acid, resulted in a decrease in CDC25B expression (Ullmannova et al. 2003).which has epigenetic effects by inhibiting histone deacetylases,

There are few imprinted genes that are found alone on chromosomes, as they frequently occur in clusters (Verona et al. 2003). Thus if an epigenetically regulated gene localizes to such an imprinted cluster region, it is possible that it may be part of a previously unidentified imprinted gene. In the data genes were found that decrease during immortalization that are known imprinted genes as well as genes that fall in imprinting regions and may be putative imprinted genes. CD81 (11p15.5), MEG3 (14q32) and NDN (15q11.2-q12) are known imprinted genes that decrease in all four immortal LFS cell lines during immortalization. Genes that have a decrease in expression during immortalization and are located near known imprinted genes (putative novel imprinting genes), include CD59 (11p13), DKFZp564J0323 (11p13-11qter), SMPD1 (11p15.4-p15.1), PHLDA2 (11p15.5), CRYAB (11q22.q23.1), IGSF4 (11q23.2), TAGLN (11q23.2), CD63 (12q12-q13), LUM (12q21.3-q22), TNFAIP2 (14q32), FBLN5 (14q32.1), KNS2 (14q32.3) and C14orf78 (14q32.33). Of particular interest is TNFAIP2, which is one of the fourteen genes that decreases during immortalization and increases after 5-aza-dC treatment in all four immortal LFS cell lines.

In summary, gene expression analysis was performed using microarrays to identify pathways critical to the process of cellular immortalization. The senescence initiating events leading to genomic instability and telomere stabilization are loss of checkpoint proteins such as p53, p21^{CIP1/WAF1}, and p16^{INK4a}. Gene profiling revealed 149 upregulated genes and 187 downregulated genes of which 14 were epigenetically downregulated in all four immortal LFS cell lines. In addition, several common pathways were involved in immortalization including the interferon pathway, genes involved in proliferation and cell cycle control, and the genes for cytoskeletal proteins.

Throughout this application, various publications, including United States patents, are referenced by author and year, and patents, by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

TABLE 7
Summary of differentially regulated genes in MDAH041 and MDAH087 immortal cell lines.

| | | | | | | | | | Common to 4 | |
|---|---|---|---|---|---|---|---|---|---|---|
| | MDAH041 | | MDAH087-N | | MDAH087-1 | | MDAH087-10 | | IM Cell Lines | |
| Comparison | Probe | Gene | Probe | Gene | Probe | Gene | Probe | Gene | Probe | Gene |
| A. IM vs PC upregulated | 1120 | 897 | 1276 | 1038 | 1544 | 1267 | 979 | 801 | 192 | 149 |
| B. IM vs PC downregulated | 1270 | 1120 | 894 | 785 | 1093 | 954 | 921 | 807 | 207 | 187 |
| C. 5-aza-dC vs IM downregulated | 928 | 803 | 869 | 717 | 816 | 713 | 484 | 408 | 49 | 46 |
| D. 5-aza-dC vs IM upregulated | 1063 | 877 | 936 | 772 | 894 | 730 | 875 | 724 | 226 | 185 |
| Genes in Sets B and D | 304 | 263 | 172 | 152 | 169 | 147 | 133 | 121 | 15 | 14 |
| Genes in Sets A and C | 175 | 159 | 355 | 284 | 262 | 233 | 109 | 90 | 2 | 2 |

Data was analyzed using Affymetrix DMT version 5;
PC: precrisis cells;
IM: immortal cells;
5-aza-dC: 5-aza-dC-treated immortal cells.
Probe: Probe ID from Affymetrix HGU95Av2 arrays.
Gene: Unigene number based on Unigene build #166.

TABLE 8
Categorization of the genes regulated in all four immortal LFS cell lines

| | Common | | p53 | Im- |
|---|---|---|---|---|
| | to 4 | IFN | regu- | print- |
| | IM Cell | regulated | lated | ed |
| Comparison | Lines | gene | gene | gene |
| A. IM vs PC upregulated | 149 | 43 | 29 | 0 |
| B. IM vs PC downregulated | 187 | 40 | 23 | 4 |
| C. 5-aza-dC vs IM downregulated | 47 | 10 | 7 | 0 |
| D. 5-aza-dC vs IM upregulated | 185 | 56 | 26 | 0 |
| Genes in Sets B and D | 14 | 2 | 1 | 0 |
| Genes in Sets A and C | 2 | 1 | 1 | 0 |

Data was analyzed using Affymetrix DMT version 5;
PC: precrisis cells;
IM: immortal cells;
5-aza-dC: 5-aza-dC-treated immortal cells.
Probe: Probe ID from Affymetrix HGU95Av2 arrays.
Gene: Unigene number based on Unigene build #166

TABLE 9
Genes differentially regulated after immortalization and
demethylation in MDAH041 and MDAH087 cells

| | | MDAH041 | | MDAH087-N | | MDAH087-1 | | MDAH087-10 | | | |
|---|---|---|---|---|---|---|---|---|---|---|---|
| Gene | LocusLink ID | IM | 5A | IM | 5A | IM | 5A | IM | 5A | CpG | LOCUS |
| CREG | 8804 | −1.6 | 2.2 | −1.5 | 1.6 | −9.3 | 4.1 | −3.3 | 2.6 | + | 1q24 |
| CYP1B1 | 1545 | −14.0 | 8.2 | −8.4 | 8.0 | −4.2 | 4.9 | −5.1 | 4.7 | + | 2p21 |
| IGFBPrP1 | 3490 | −2.0 | 2.5 | −4.5 | 3.0 | −10.1 | 1.9 | −4.9 | 1.7 | + | 4q12 |
| CLTB | 1212 | −1.3 | 1.5 | −1.9 | 2.4 | −4.0 | 2.1 | −2.4 | 1.8 | + | 5q35 |
| KIAA1750 | 85453 | −27.8 | 2.9 | −18.8 | 5.1 | −3.7 | 3.3 | −15.2 | 9.1 | + | 8q22.1 |
| FLJ14675 | 84909 | −2.7 | 3.8 | −6.0 | 2.3 | −2.0 | 1.8 | −2.2 | 1.7 | + | 9q22 |
| OPTN | 10133 | −3.0 | 2.6 | −1.8 | 1.8 | −3.6 | 1.6 | −2.1 | 1.3 | + | 10p14 |
| HPS5 | 11234 | −1.6 | 1.6 | −1.6 | 2.2 | −1.7 | 1.7 | −2.4 | 1.5 | + | 11p14 |
| HTATIP2 | 10553 | −17.7 | 5.6 | −6.6 | 3.7 | −2.9 | 2.0 | −5.5 | 2.4 | + | 11p15.1 |
| HSPA2 | 3306 | −2.0 | 1.6 | −12.4 | 10.9 | −5.2 | 5.3 | −5.2 | 6.4 | − | 14q24.1 |
| TNFAIP2 | 7127 | −6.0 | 5.4 | −9.3 | 5.4 | −4.3 | 3.1 | −3.7 | 2.1 | − | 14q32 |
| ALDH1A3 | 220 | −2.7 | 2.8 | −2.3 | 3.2 | −8.0 | 2.3 | −4.4 | 3.8 | + | 15q26.3 |
| MAP1LC3B | 81631 | −1.6 | 1.7 | −2.5 | 2.5 | −2.0 | 3.1 | −1.3 | 1.8 | + | 16q24.2 |
| SERPINB2 | 5055 | −1.3 | 4.2 | −3.0 | 10.7 | −2.6 | 8.8 | −4.3 | 7.0 | − | 18q21.3 |

Fold change of gene expression level were processed in Affymetrix DMT, version 5.
Fold changes for genes with multiple probes were averaged;
5A: upregulation in 5-aza-dC-treated immortal cells versus untreated immortal cells;
IM: down-regulation in immortal cells versus precrisis cells;

TABLE 10
Comparison of changes in expression in microarrays and by Q-RT-PCR analysis

| | | MDAH041 | | | | MDAH087-N | | | |
|---|---|---|---|---|---|---|---|---|---|
| Gene | | IM vs PC | | IM vs 5-aza-dC | | IM vs PC | | IM vs 5-aza-dC | |
| Symbol | LocusLinkID | MA | QP | MA | QP | MA | QP | MA | QP |
| ALDH1A3 | 220 | −2.7 | −13.94 | 2.8 | 7.84 | −2.3 | −4.31 | 3.2 | 11.51 |
| CLTB | 1212 | −1.3 | −7.51 | 1.5 | 2.04 | −1.9 | −6.11 | 2.4 | 7.62 |
| CREG | 8804 | −1.65 | −8.50 | 2.21 | 2.88 | −1.54 | −2.75 | 1.55 | 4.80 |
| HSPA2 | 3306 | −2.0 | 0.60 | 1.6 | 2.74 | −12.4 | −144.35 | 10.9 | 46.6 |
| IGFBPrP1 | 3490 | −2.00 | −8.80 | 2.51 | 12.27 | −4.47 | −27.15 | 3.02 | 5.34 |
| KIAA1750 | 85453 | −27.76 | −3.78 | 2.88 | 3.86 | −18.81 | −403.99 | 5.10 | 28.43 |
| OPTN | 10133 | −3.0 | −15.30 | 2.6 | 9.21 | −1.8 | −4.68 | 1.8 | 6.36 |
| SERPINB2 | 5055 | −1.3 | −42.08 | 4.2 | 12.90 | −3.0 | −6.52 | 10.7 | 66.72 |
| TNFAIP2 | 7127 | −5.97 | −18.90 | 5.41 | 6.10 | −9.34 | −32.01 | 5.41 | 39.56 |
| STAT1α | 6772 | −1.57 | −16.21 | 2.92 | 119.85 | −1.3 | −4.39 | 1.99 | 9.05 |
| IGFBP3 | 3486 | −2.24 | 0.24 | 2.48 | 8.29 | −6.25 | −111.11 | −1.03 | 2.82 |
| IGFBP4 | 3487 | −21.66 | −50.58 | 3.72 | 7.00 | −2.89 | −24.21 | 1.84 | 4.82 |

| | | MDAH087-1 | | | | MDAH087-10 | | | |
|---|---|---|---|---|---|---|---|---|---|
| Gene | | IM vs PC | | IM vs 5-aza-dC | | IM vs PC | | IM vs 5-aza-dC | |
| Symbol | LocusLinkID | MA | QP | MA | QP | MA | QP | MA | QP |
| ALDH1A3 | 220 | −8.0 | −42.13 | 2.3 | 69.28 | −4.4 | −10.42 | 3.8 | 9.71 |
| CLTB | 1212 | −4.0 | −3.66 | 2.1 | 3.08 | −2.4 | −5.4 | 1.8 | 7.5 |
| CREG | 8804 | −9.32 | −8.34 | 4.10 | 4.93 | −3.28 | −3.11 | 2.56 | 4.49 |
| HSPA2 | 3306 | −5.2 | −7.21 | 5.3 | 24.43 | −5.2 | −17.7 | 6.4 | 3.65 |
| IGFBPrP1 | 3490 | −10.15 | −52.02 | 1.93 | 1.93 | −4.86 | −41.85 | 1.69 | 4.10 |
| KIAA1750 | 85453 | −3.72 | −10.83 | 3.28 | 5.23 | −15.24 | −30.42 | 9.09 | 31.41 |
| OPTN | 10133 | −3.6 | −8.65 | 1.6 | 30.54 | −2.1 | −1553.76 | 1.3 | 431.43 |
| SERPINB2 | 5055 | −2.6 | −5.19 | 8.8 | 22.63 | −4.3 | −12.17 | 7.0 | 58.24 |
| TNFAIP2 | 7127 | −4.34 | −8.44 | 3.08 | 7.28 | −3.69 | −5.14 | 2.14 | 3.72 |
| STAT1α | 6772 | −1.51 | −3.94 | 1.05 | 1.56 | −2.49 | −15.23 | 1.42 | 3.23 |
| IGFBP3 | 3486 | −5.76 | −46.68 | −1.58 | −2.61 | −6.80 | −83.13 | −2.66 | −1.40 |
| IGFBP4 | 3487 | −2.15 | −10.59 | 1.08 | −0.03 | −4.37 | −34.80 | 1.9 | 3.72 |

Average fold change of gene expression after immortalization (IM versus PC) and after treatment with 5-aza-dC (IM versus 5-aza-dC)
MA: fold change microarray;
QP: fold change Q-RT-PCR

TABLE 11

Primers used in Q-RT-PCR

| | | |
|---|---|---|
| Gene | Forward prime¹ | Reverse primer¹ |

| | | |
|---|---|---|
| ALDH1A3 | GCCAGGGTCTTTGTGGATTG | AGCTCTCTGGGCTATTGATTCTG |
| | | T |
| CLTB | AACAACCGGATCGCTGACA | CCTCCTTGGATTCCTTCACG |
| CREG | CAGCTTCAGCCAGGGACAAA | GGGCAGTTGAGGAAGCCTTAG |
| GAPDH | ATCAAGAAGGTGGTGAAGCAG | TGTCGCTGTTGAAGTCAGAGG |
| HSPA2 | ACCGAAACCAGATGGCAGAG | GGACCACCTTGGTAAAGTTTGCT |
| IGFBP3 | AACTGTGGCCATGACTGAGGA | CTCCCTGAGCCTGACTTTGC |
| IGFBP4 | ACCCACTCCCAAAGCTCAGA | TGCCAGCCAACCAAGCA |
| IGFBPrP1 | GCCATGCATCCAATTCCC | TCGGCACCTTCACCTTTTTT |
| KIAA1750 | TATGGTCAACCTGGTTTCATCTGT | CTCCCAAAGTAGTCACGGTTGC |
| OPTN | GAGAAGGCTCTGGCTTCCAA | GAGCCCTGAGGATGGTCATG |
| SERPINB2 | GGACGGGCCAATTTCTCAG | CTTCAGTGCCCTCCTCATTCA |
| TNFAIP2 | TAGCCTCCTAAAGTGCTGGGAT | TCTCTGGGTAGGCGCAATGT |

¹All primers have a 5′-3′ orientation

Table 12 Summary of differentially regulated genes/probes in MDAH041 and MDAH087 cells during immortalization.

TABLE 12a
192 probes upregulated IM vs PC (see Table 1A)

| Symbol | Affymetrix HGU95Av2 Probe ID | LocusLink | Average Singal Log₂ | Fold Change | Locus |
|---|---|---|---|---|---|
| AK3 | 32331_at | 205 | 1.66 | 3.15 | 1p31.3 |
| ALDH6A1 | 32676_at | 4329 | 1.30 | 2.46 | 14q24.3 |
| ANAPC7 | 37171_at | 51434 | 0.83 | 1.78 | 12q13.12 |
| APEX1 | 2025_s_at | 328 | 0.83 | 1.78 | 14q11.2-q12 |
| APOBEC3B | 39230_at | 9582 | 1.33 | 2.52 | 22q13.1-q13.2 |
| ARF6 | 37984_s_at | 382 | 0.91 | 1.88 | 14q21.3 |
| ARHGEF2 | 40100_at | 9181 | 0.72 | 1.65 | 1q21-q22 |
| ASPH | 37528_at | 444 | 1.16 | 2.23 | 8q12.1 |
| ATP2B1 | 37661_at | 490 | 1.59 | 3.01 | 12q21-q23 |
| ATP5S | 40027_at | 27109 | 1.09 | 2.13 | 14q22.1 |
| BAX | 2065_s_at | 581 | 0.79 | 1.73 | 19q13.3-q13.4 |
| BCAT1 | 38201_at | 586 | 2.06 | 4.18 | 12pter-q12 |
| BCAT2 | 41111_at | 587 | 0.77 | 1.70 | 19q13 |
| BIN1 | 132238_at | 274 | 1.04 | 2.05 | 2q14 |
| BOP1 | 35615_at | 23246 | 1.24 | 2.37 | 8q24.3 |
| BSG | 36162_at | 682 | 1.00 | 2.00 | 19p13.3 |
| BTBD2 | 35155_at | 55643 | 1.81 | 3.50 | 19p13.3 |
| C6orf69 | 35001_at | 222658 | 1.13 | 2.19 | 6p21.31 |
| C6orf69 | 35002_g_at | 222658 | 2.17 | 4.51 | 6p21.31 |
| CDC20 | 38414_at | 991 | 1.06 | 2.09 | 1p34.1 |
| CDC25B | 1347_at | 994 | 0.64 | 1.56 | 20p13 |
| CDKN3 | 1599_at | 1033 | 1.05 | 2.07 | 14q22 |
| CELSR3 | 40020_at | 1951 | 1.00 | 2.00 | 3p24.1-p21.2 |
| CENPB | 37931_at | 1059 | 0.98 | 1.97 | 20p13 |
| CHC1 | 37927_at | 1104 | 1.04 | 2.05 | 1p36.1 |
| CHD1 | 39231_at | 1105 | 0.95 | 1.93 | 5q15-q21 |
| CKS2 | 40690_at | 1164 | 1.02 | 2.02 | 9q22 |
| COX11 | 34723_at | 1353 | 1.17 | 2.25 | 17q22 |
| CSNK1G2 | 446_at | 1455 | 1.33 | 2.51 | 19p13.3 |
| CSNK2A1 | 40258_at | 1457 | 0.89 | 1.86 | 20p13 |
| CSNK2A1 | 594_s_at | 1457 | 0.96 | 1.95 | 20p13 |
| CSNK2B | 32843_s_at | 1460 | 1.00 | 1.99 | 6p21-p12 |
| D10S170 | 37162_at | 8030 | 0.83 | 1.78 | 10q21 |
| DDX27 | 33650_at | 55661 | 0.99 | 1.99 | 20q13.13 |
| DDX3X | 826_at | 1654 | 1.06 | 2.09 | Xp11.3-p11.23 |
| DKFZP564J157 | 35745_f_at | 54458 | 0.93 | 1.90 | 12q12 |
| DKFZP564J157 | 35746_r_at | 54458 | 0.76 | 1.70 | 12q12 |
| DKFZP564O243 | 41018_at | 25864 | 0.80 | 1.74 | 3p21.1 |
| DLG7 | 37231_at | 9787 | 1.02 | 2.03 | 14q22.2 |
| DPYD | 38220_at | 1806 | 1.10 | 2.14 | 1p22 |
| E2-EPF | 893_at | 27338 | 1.41 | 2.65 | 19q13.43 |
| E2F4 | 1703_g_at | 1874 | 1.24 | 2.36 | 16q21-q22 |
| EGFR | 1537_at | 1956 | 2.97 | 7.86 | 7p12 |
| EMP1 | 1321_s_at | 2012 | 2.58 | 5.96 | 12p12.3 |
| ERF | 38996_at | 2077 | 1.12 | 2.17 | 19q13 |
| EWSR1 | 423_at | 2130 | 1.11 | 2.17 | 22q12.2 |
| FBL | 39173_at | 2091 | 0.91 | 1.88 | 19q13.1 |
| FNTB | 37488_at | 2342 | 0.89 | 1.86 | 14q23-q24 |
| FUS | 39180_at | 2521 | 1.00 | 2.00 | 16p11.2 |
| FZD2 | 36799_at | 2535 | 1.01 | 2.01 | 17q21.1 |
| FZD2 | 628_at | 2535 | 1.16 | 2.23 | 17q21.1 |
| GAPD | AFFX-HUMGAPDH/M33197_5_at | 2597 | 0.42 | 1.34 | 12p13 |
| GG2-1 | 33243_at | 25816 | 1.17 | 2.25 | 5q23.1 |
| GNA13 | 1139_at | 10672 | 1.61 | 3.05 | 17q24.3 |
| GNS | 36262_at | 2799 | 1.31 | 2.48 | 12q14 |
| GNS | 36263_g_at | 2799 | 2.04 | 4.12 | 12q14 |
| GUSB | 33308_at | 2990 | 0.60 | 1.51 | 7q21.11 |
| H1F0 | 33386_at | 3005 | 0.91 | 1.88 | 22q13.1 |
| H1FX | 318_at | 8971 | 1.75 | 3.36 | 3q21.3 |
| H1FX | 319_g_at | 8971 | 1.06 | 2.09 | 3q21.3 |
| H2AFY | 36576_at | 9555 | 0.67 | 1.59 | 5q31.3-q32 |
| HAN11 | 38171_at | 10238 | 0.76 | 1.69 | 17q24.2 |
| HAN11 | 41591_at | 10238 | 0.90 | 1.86 | 17q24.2 |
| HDAC1 | 476_s_at | 3065 | 0.92 | 1.90 | 1p34 |
| IDH3B | 40110_at | 3420 | 0.79 | 1.73 | 20p13 |
| IER3 | 1237_at | 8870 | 1.46 | 2.75 | 6p21.3 |
| IGF1R | 34718_at | 3480 | 0.86 | 1.81 | 15q25-q26 |
| ILF3 | 40845_at | 3609 | 1.33 | 2.51 | 19p13.2 |
| ILF3 | 40846_g_at | 3609 | 0.91 | 1.88 | 19p13.2 |
| IMPDH2 | 36624_at | 3615 | 0.77 | 1.70 | 3p21.2 |
| KCNG1 | 37498_at | 3755 | 0.98 | 1.97 | 20q13 |
| KCNMA1 | 40737_at | 3778 | 0.84 | 1.79 | 10q22-q23 |
| KIAA0186 | 39677_at | 9837 | 1.24 | 2.36 | 20P11.21 |
| KIAA0528 | 35252_at | 9847 | 1.04 | 2.05 | 12p12.2 |
| KIAA0863 | 37837_at | 22850 | 1.43 | 2.70 | 18q23 |
| KPNB2 | 40463_at | 3842 | 0.82 | 1.76 | 5q13.2 |
| LDB2 | 36065_at | 9079 | 1.23 | 2.34 | 4p16 |
| LMNB1 | 37985_at | 4001 | 1.35 | 2.54 | 5q23.3-q31.1 |
| LMNB2 | 36987_at | 84823 | 1.03 | 2.05 | 19p13.3 |
| MADH5 | 1013_at | 4090 | 0.96 | 1.94 | 5q31 |
| MADH5 | 39926_at | 4090 | 1.09 | 2.13 | 5q31 |
| MAP1B | 39531_at | 4131 | 1.47 | 2.76 | 5q13 |
| MAP1B | 41373_s_at | 4131 | 1.97 | 3.92 | 5q13 |
| MAPK1 | 976_s_at | 5594 | 0.82 | 1.77 | 22q11.2 |
| MAPKAPK2 | 1439_s_at | 9261 | 2.18 | 4.54 | 1q32 |
| MAT2A | 32571_at | 4144 | 0.88 | 1.84 | 2p11.2 |
| MAX | 1981_s_at | 4149 | 1.54 | 2.91 | 14q23 |
| MAZ | 1764_s_at | 4150 | 2.07 | 4.20 | 16p11.2 |
| MAZ | 32553_at | 4150 | 0.59 | 1.51 | 16p11.2 |
| MET | 35684_at | 4233 | 1.15 | 2.23 | 7q31 |
| MRPS12 | 33214_at | 6183 | 0.78 | 1.72 | 19q13.1-q13.2 |
| MSC | 35992_at | 9242 | 1.87 | 3.64 | 8q21 |
| MTHFD1 | 673_at | 4522 | 1.39 | 2.62 | 14q24 |
| MTHFD1 | 674_g_at | 4522 | 1.01 | 2.01 | 14q24 |
| MYC | 1827_s_at | 4609 | 1.14 | 2.20 | 8q24.12-q24.13 |
| MYC | 1973_s_at | 4609 | 1.63 | 3.10 | 8q24.12-q24.13 |
| MYC | 37724_at | 4609 | 1.31 | 2.48 | 8q24.12-q24.13 |
| MYO10 | 35362_at | 4651 | 0.80 | 1.74 | 5p15.1-p14.3 |
| NDUFV1 | 37329_at | 4723 | 0.79 | 1.73 | 11q13 |
| NOL1 | 1979_s_at | 4839 | 1.31 | 2.49 | 12p13 |
| NOL5A | 34882_at | 10528 | 0.82 | 1.77 | 20p13 |
| NR1D2 | 35705_at | 9975 | 1.43 | 2.69 | 3p24.1 |
| NR1H2 | 518_at | 7376 | 0.93 | 1.91 | 19q13.3-19q13.3 |
| NRAS | 1539_at | 4893 | 1.38 | 2.60 | 1P13.2 |
| NRP1 | 36836_at | 8829 | 1.54 | 2.91 | 10p12 |
| OSBP | 41313_at | 5007 | 0.92 | 1.89 | 11q12-q13 |
| PAI-RBP1 | 40440_at | 26135 | 0.97 | 1.96 | 1p31-p22 |
| PDK1 | 36386_at | 5163 | 1.61 | 3.06 | 2q31.1 |
| PES1 | 41869_at | 23481 | 0.87 | 1.82 | 22q12.1 |
| PGK1 | 31488_s_at | 5230 | 1.29 | 2.44 | Xq13 |
| PITPNB | 353_at | 23760 | 1.51 | 2.86 | 22q12.1 |
| PLD3 | 36151_at | 23646 | 1.04 | 2.06 | 19q13.2 |
| PLK | 37228_at | 5347 | 1.08 | 2.12 | 16p12.3 |
| PLTP | 40081_at | 5360 | 1.06 | 2.09 | 20q12-q13.1 |
| PPP2R1A | 922_at | 5518 | 0.63 | 1.54 | 19q13.41 |
| PPP4C | 382_at | 5531 | 0.59 | 1.50 | 16p12-16p11 |
| PPP5C | 391_at | 5536 | 1.18 | 2.26 | 19q13.3 |
| PPP5C | 392_g_at | 5536 | 1.72 | 3.30 | 19q13.3 |
| PPT1 | 34774_at | 5538 | 1.10 | 2.14 | 1p32 |
| PRIM1 | 798_at | 5557 | 1.75 | 3.35 | 12q13 |
| PRIM2A | 122_at | 5558 | 0.90 | 1.86 | 6p12-p11.1 |
| PRKCA | 32304_at | 5578 | 1.05 | 2.07 | 17q22-q23.2 |
| PRKDC | 2012_s_at | 5591 | 1.20 | 2.30 | 8q11 |
| PROSC | 40545_at | 11212 | 1.09 | 2.13 | 8p11.2 |
| PRRX1 | 40126_at | 5396 | 1.13 | 2.19 | 1q24 |
| PTMS | 40580_r_at | 5763 | 1.01 | 2.02 | 12p13 |
| PTPN11 | 1870_at | 5781 | 1.77 | 3.41 | 12q24 |
| PTRF | 36369_at | 284119 | 1.95 | 3.87 | 17q21.31 |
| RAB5A | 600_at | 5868 | 0.94 | 1.92 | 3p24-p22 |
| RAD21 | 38114_at | 5885 | 0.77 | 1.70 | 8q24 |
| RAF1 | 1917_at | 5894 | 0.85 | 1.80 | 3p25 |
| RALY | 36125_s_at | 22913 | 0.98 | 1.97 | 20q11.21-q11.23 |
| RBMX | 39731_at | 27316 | 0.78 | 1.72 | Xq26 |
| REA | 37364_at | 11331 | 0.75 | 1.68 | 12p13 |
| RELA | 1045_s_at | 5970 | 1.84 | 3.59 | 11q13 |
| RFC5 | 38863_at | 5985 | 1.16 | 2.23 | 12q24.2-q24.3 |
| RRP4 | 32974_at | 23404 | 1.17 | 2.24 | 9q34 |
| SCG2 | 36924_r_at | 7857 | 2.44 | 5.43 | 2q35-q36 |
| SFRS3 | 351_f_at | 6428 | 1.05 | 2.08 | 6p21 |
| SIP1 | 41363_at | 8487 | 1.22 | 2.34 | 14q13 |
| SLC25A1 | 38998_g_at | 6576 | 0.62 | 1.53 | 22q11.21 |
| SMC2L1 | 37502_at | 10592 | 1.18 | 2.27 | 9q31.2 |
| SNRPB | 38455_at | 6628 | 0.99 | 1.99 | 20p13 |
| SNRPB | 38456_s_at | 6628 | 0.54 | 1.46 | 20p13 |
| SQLE | 35839_at | 6713 | 0.85 | 1.80 | 8q24.1 |
| STK6 | 34851_at | 6790 | 0.99 | 1.98 | 20q13.2-q13.3 |
| TCOF1 | 40596_at | 6949 | 0.83 | 1.77 | 5q32-q33.1 |
| TFRC | AFFX-HUMTFRR/M11507_3_at | 7037 | 1.60 | 3.03 | 3q26.2-qter |
| TFRC | AFFX-HUMTFRR/M11507_5_at | 7037 | 2.00 | 4.00 | 3q26.2-qter |
| TFRC | AFFX-HUMTFRR/M11507_M_at | 7037 | 2.22 | 4.66 | 3q26.2-qter |
| TMPO | 32682_at | 7112 | 2.20 | 4.59 | 12q22 |
| TMPO | 32683_at | 7112 | 1.26 | 2.39 | 12q22 |
| TOMM40 | 35620_at | 10452 | 1.36 | 2.57 | 19q13 |
| TOP1 | 1710_s_at | 7150 | 1.80 | 3.48 | 20q12-q13.1 |
| TOP2B | 1581_s_at | 7155 | 1.42 | 2.68 | 3p24 |
| TPX2 | 39109_at | 22974 | 0.85 | 1.80 | 20q11.2 |
| TRIO | 40581_at | 7204 | 1.48 | 2.80 | 5p15.1-p14 |
| TRIO | 40792_s_at | 7204 | 1.49 | 2.80 | 5p15.1-p14 |
| TULP3 | 31943_g_at | 7289 | 1.13 | 2.19 | 12p13.3 |
| TXNDC | 34768_at | 81542 | 0.90 | 1.86 | 14q22.1 |
| UBE2C | 1651_at | 11065 | 1.08 | 2.11 | 20q13.12 |
| UBE2M | 33781_s_at | 9040 | 0.88 | 1.84 | 19q13.43 |
| UBTF | 38795_s_at | 7343 | 1.10 | 2.14 | 17q21.3 |
| VEGF | 1953_at | 7422 | 1.08 | 2.11 | 6p12 |
| VEGF | 36100_at | 7422 | 1.04 | 2.06 | 6p12 |
| VEGF | 36101_s_at | 7422 | 3.52 | 11.49 | 6p12 |
| WNT5A | 1669_at | 7474 | 1.09 | 2.13 | 3p21-p14 |
| WNT5A | 31862_at | 7474 | 1.04 | 2.05 | 3p21-p14 |
| — | 1196_at | — | 1.49 | 2.80 | — |
| — | 1258_s_at | — | 1.04 | 2.05 | — |
| — | 1609_g_at | — | 1.34 | 2.54 | — |
| — | 1635_at | — | 0.92 | 1.89 | — |
| — | 1750_at | — | 0.97 | 1.96 | — |
| — | 1753_s_at | — | 1.44 | 2.70 | — |
| — | 1812_s_at | — | 1.16 | 2.24 | — |
| — | 1942_s_at | — | 0.95 | 1.93 | — |
| — | 2049_s_at | — | 2.38 | 5.19 | — |
| — | 31510_s_at | — | 0.57 | 1.49 | — |
| — | 33761_s_at | 376645 | 1.22 | 2.32 | 1q21.2 |
| — | 34069_s_at | — | 0.70 | 1.62 | — |
| — | 34374_g_at | — | 0.73 | 1.65 | — |
| — | 38022_s_at | — | 2.89 | 7.40 | — |
| — | 39470_at | — | 1.76 | 3.38 | — |
| — | 39537_at | — | 2.40 | 5.28 | — |
| — | 39560_at | — | 2.23 | 4.69 | — |
| — | 39694_at | — | 0.94 | 1.92 | — |
| — | 40608_at | 376135 | 2.39 | 5.25 | 12q15 |
| — | 434_at | — | 3.70 | 13.02 | — |
| — | 625_at | — | 1.29 | 2.45 | — |
| — | 910_at | — | 1.74 | 3.33 | — |
| — | 919_at | — | 1.32 | 2.50 | — |
| — | 953_g_at | — | 1.23 | 2.34 | — |

TABLE 12b
207 genes/probes downregulated IM vs PC (see Table 1B)

| | Affymetrix HGU95Av2 | | Average | Fold | |
|---|---|---|---|---|---|
| Symbol | Probe ID | LocusLink | Signal Log₂ | Change | Locus |
| ABAT | 33446_at | 18 | −1.92 | −3.77 | 16p13.2 |
| ACTA2 | 32755_at | 59 | −2.48 | −5.57 | 10q23.3 |
| ACTC | 39063_at | 70 | −6.09 | −67.90 | 15q11-q14 |
| ACTR1A | 40052_at | 10121 | −0.58 | −1.50 | 10q24.33 |
| ADD1 | 32145_at | 118 | −0.48 | −1.40 | 4p16.3 |
| ALDH1A3 | 36686_at | 220 | −1.94 | −3.82 | 15q26.3 |
| ANXA11 | 36637_at | 311 | −0.97 | −1.96 | 10q23 |
| APM2 | 32527_at | 10974 | −5.01 | −32.31 | 10q23.31 |
| ARPC1B | 39043_at | 10095 | −1.10 | −2.14 | 7q22.1 |
| ASS | 40541_at | 445 | −1.76 | −3.38 | 9q34.1 |
| ATOX1 | 41776_at | 475 | −1.00 | −2.00 | 5q32 |
| ATP2B4 | 40913_at | 493 | −0.88 | −1.84 | 1q25-q32 |
| ATP6V0E | 33875_at | 8992 | −1.09 | −2.13 | 5q35.2 |
| BPAG1 | 32780_at | 667 | −0.58 | −1.50 | 6p12-p11 |
| C14orf78 | 36497_at | 113146 | −2.03 | −4.08 | 14q32.33 |
| C21orf80 | 34287_at | 23275 | −0.74 | −1.67 | 21q22.3 |
| C6orf109 | 38697_at | 25844 | −0.77 | −1.70 | 6p21.1 |
| C6orf32 | 37112_at | 9750 | −4.05 | −16.61 | 6p22.3-p21.32 |
| CAP2 | 33405_at | 10486 | −1.34 | −2.53 | 6p22.3 |
| CAPG | 38391_at | 822 | −2.50 | −5.65 | 2cen-q24 |
| CCND1 | 2020_at | 595 | −1.31 | −2.48 | 11q13 |
| CCND1 | 38418_at | 595 | −1.34 | −2.53 | 11q13 |
| CD59 | 39351_at | 966 | −1.01 | −2.02 | 11p13 |
| CD63 | 37003_at | 967 | −0.76 | −1.69 | 12q12-q13 |
| CD81 | 35282_r_at | 975 | −1.11 | −2.16 | 11p15.5 |
| CD97 | 35625_at | 976 | −0.70 | −1.63 | 19p13 |
| CD99 | 41138_at | 4267 | −1.35 | −2.55 | Xp22.32 |
| CDKN1A | 2031_s_at | 1026 | −2.02 | −4.04 | 6p21.2 |
| CH25H | 32363_at | 9023 | −3.66 | −12.60 | 10q23 |
| CLECSF2 | 40698_at | 9976 | −2.73 | −6.62 | 12p13-p12 |
| CLIC1 | 36131_at | 1192 | −0.62 | −1.54 | 6p22.1-p21.2 |
| CLTB | 32523_at | 1212 | −1.16 | −2.24 | 4q2-q3 |
| CNN1 | 34203_at | 1264 | −3.39 | −10.50 | 19p13.2-p13.1 |
| COL4A1 | 39333_at | 1282 | −2.82 | −7.04 | 13q34 |
| COL4A2 | 36659_at | 1284 | −1.93 | −3.82 | 13q34 |
| COX7A1 | 39031_at | 1346 | −6.61 | −97.34 | 19q13.1 |
| CREG | 35311_at | 8804 | −1.57 | −2.97 | 1q24 |
| CRIP1 | 33232_at | 1396 | −2.63 | −6.18 | 7q11.23 |
| CRYAB | 32242_at | 1410 | −3.95 | −15.41 | 11q22.3-q23.1 |
| CRYAB | 32243_g_at | 1410 | −3.84 | −14.32 | 11q22.3-q23.1 |
| CSRP1 | 38700_at | 1465 | −1.14 | −2.21 | 1q32 |
| CXCL12 | 32666_at | 6387 | −3.40 | −10.56 | 10q11.1 |
| CYB5R1 | 35329_at | 51706 | −1.08 | −2.11 | 1p36.13-q41 |
| CYBA | 35807_at | 1535 | −1.58 | −2.99 | 16q24 |
| CYP1B1 | 40071_at | 1545 | −2.68 | −6.42 | 2p21 |
| CYP1B1 | 859_at | 1545 | −2.82 | −7.08 | 2p21 |
| DEGS | 33337_at | 8560 | −0.94 | −1.92 | 1q42.12 |
| DHX29 | 39140_at | 54505 | −0.86 | −1.81 | 5q11.2 |
| DIA1 | 36668_at | 1727 | −0.69 | −1.62 | 22q13.31-qter |
| DKFZP564B167 | 37000_at | 25874 | −1.19 | −2.28 | 1q24 |
| DKFZp564I1922 | 36861_at | 25878 | −3.50 | −11.31 | Xp22.33 |
| DKK1 | 35977_at | 22943 | −1.94 | −3.84 | 10q11.2 |
| DNASE1L1 | 37214_g_at | 1774 | −1.55 | −2.92 | Xq28 |
| DSP | 36133_at | 1832 | −3.91 | −15.04 | 6p24 |
| DUSP14 | 38272_at | 11072 | −1.05 | −2.07 | 17q12 |
| ECM1 | 37600_at | 1893 | −1.77 | −3.40 | 1q21 |
| ELN | 39098_at | 2006 | −3.36 | −10.23 | 7q11.23 |
| EMS1 | 39861_at | 2017 | −1.35 | −2.56 | 11q13 |
| ENG | 32562_at | 2022 | −0.93 | −1.90 | 9q33-q34.1 |
| EPB41L3 | 41385_at | 23136 | −4.68 | −25.66 | 18p11.32 |
| ETHE1 | 36170_at | 23474 | −1.26 | −2.40 | 19q13.32 |
| FAM20B | 35318_at | 9917 | −1.10 | −2.15 | 1p36.13-q41 |
| FAM8A1 | 38318_at | 51439 | −1.11 | −2.16 | 6p22-p23 |
| FARP1 | 32148_at | 10160 | −2.27 | −4.82 | 13q32.2-q32.3 |
| FBLN5 | 39038_at | 10516 | −1.70 | −3.24 | 14q32.1 |
| FBXO9 | 38990_at | 26268 | −0.80 | −1.74 | 6p12.3-p11.2 |
| FEZ1 | 37743_at | 9638 | −3.53 | −11.58 | 11q24.2 |
| FEZ2 | 38651_at | 9637 | −0.84 | −1.79 | 2p21 |
| FGF7 | 1466_s_at | 2252 | −2.03 | −4.09 | 15q15-q21.1 |
| FKBP9 | 38761_s_at | 11328 | −1.20 | −2.30 | 7p11.1 |
| FLJ10055 | 33193_at | 55062 | −0.84 | −1.78 | 17q24.3 |
| FLJ10849 | 35181_at | 55752 | −1.27 | −2.40 | 4q21.22 |
| FLJ14675 | 41207_at | 84909 | −1.53 | −2.89 | 9q22.33 |
| FLJ31737 | 41013_at | 196740 | −1.20 | −2.30 | 10q11.23 |
| FNBP1 | 40468_at | 23048 | −0.98 | −1.97 | 9q34 |
| GABARAPL1 | 35785_at | 23710 | −2.18 | −4.52 | 12p13.31 |
| GAF1 | 33882_at | 26056 | −0.57 | −1.48 | 2p13-p12 |
| GOLGA3 | 34861_at | 2802 | −0.80 | −1.74 | 12q24.33 |
| GSN | 32612_at | 2934 | −1.27 | −2.42 | 9q33 |
| GUK1 | 905_at | 2987 | −0.64 | −1.56 | 1q32-q41 |
| HOXD4 | 38294_at | 3233 | −1.04 | −2.05 | 2q31.1 |
| HPS5 | 35223_at | 11234 | −0.81 | −1.76 | 11p14 |
| HSPA2 | 36925_at | 3306 | −2.34 | −5.07 | 14q24.1 |
| HTATIP2 | 38824_at | 10553 | −2.71 | −6.55 | 11p15.1 |
| IGFBP3 | 37319_at | 3486 | −2.27 | −4.84 | 7p13-p12 |
| IGFBP4 | 1737_s_at | 3487 | −2.30 | −4.93 | 17q12-q21.1 |
| IGFBP4 | 39781_at | 3487 | −2.41 | −5.33 | 17q12-q21.1 |
| IGFBP7 | 2062_at | 3490 | −2.20 | −4.58 | 4q12 |
| IGSF4 | 35829_at | 23705 | −2.39 | −5.24 | 11q23.2 |
| ISLR | 38636_at | 3671 | −2.34 | −5.05 | 15q23-q24 |
| ITGA1 | 37484_at | 3672 | −1.90 | −3.72 | 5q11.2 |
| ITGA7 | 36892_at | 3679 | −2.00 | −3.99 | 12q13 |
| KCTD12 | 38972_at | 115207 | −2.20 | −4.59 | 13q22.1 |
| KIAA0033 | 41129_at | 23027 | −0.92 | −1.90 | 11p15.3 |
| KIAA0367 | 33442_at | 23273 | −2.16 | −4.47 | 9q21.31 |
| KIAA0711 | 36453_at | 9920 | −4.13 | −17.53 | 8p23.3 |
| KIAA0746 | 41585_at | 23231 | −2.82 | −7.07 | 4p15.31 |
| KIAA1026 | 39615_at | 23254 | −1.36 | −2.57 | 1p36.13 |
| KIAA1128 | 37617_at | 54462 | −0.81 | −1.75 | 10q23.2 |
| KIAA1279 | 40831_at | 26128 | −0.82 | −1.77 | 10q22.1 |
| KIAA1750 | 32730_at | 85453 | −3.71 | −13.12 | 8q22.1 |
| KNS2 | 39057_at | 3831 | −0.69 | −1.62 | 14q32.3 |
| LITAF | 37025_at | 9516 | −1.87 | −3.66 | 16p13.3-p12 |
| LOC92689 | 38643_at | 92689 | −0.61 | −1.53 | 4p14 |
| LRP10 | 34409_at | 26020 | −0.80 | −1.74 | 14q11.2 |
| LUM | 38038_at | 4060 | −1.59 | −3.02 | 12q21.3-q22 |
| MAP1A | 35917_at | 4130 | −1.30 | −2.46 | 15q13-qter |
| MAP1B | 38396_at | 4131 | −0.65 | −1.57 | 5q13 |
| MAP1LC3B | 39370_at | 81631 | −0.85 | −1.80 | 16q24.2 |
| MAP2K3 | 1622_at | 5606 | −1.32 | −2.50 | 17q11.2 |
| ME1 | 31824_at | 4199 | −1.38 | −2.61 | 6q12 |
| MEG3 | 39026_r_at | 55384 | −0.98 | −1.97 | 14q32 |
| MFGE8 | 34403_at | 4240 | −3.09 | −8.52 | 15q26 |
| MGMT | 2052_g_at | 4255 | −1.78 | −3.44 | 10q26 |
| MRCL3 | 33447_at | 10627 | −1.77 | −3.42 | 18p11.31 |
| M-RIP | 38730_at | 23164 | −0.71 | −1.64 | 17p11.2 |
| MYL9 | 39145_at | 10398 | −1.49 | −2.81 | 20q11.23 |
| NDN | 36073_at | 4692 | −4.51 | −22.82 | 15q11.2-q12 |
| NID | 35366_at | 4811 | −1.25 | −2.38 | 1q43 |
| NME4 | 39089_at | 4833 | −0.85 | −1.80 | 16p13.3 |
| NOTCH3 | 38750_at | 4854 | −3.66 | −12.62 | 19p13.2-p13.1 |
| NQO1 | 38066_at | 1728 | −1.71 | −3.27 | 16q22.1 |
| NT5C2 | 31794_at | 22978 | −1.13 | −2.18 | 10q24.33 |
| OPTN | 41742_s_at | 10133 | −1.34 | −2.53 | 10p14 |
| OPTN | 41743_i_at | 10133 | −1.19 | −2.28 | 10p14 |
| OPTN | 41744_at | 10133 | −1.95 | −3.85 | 10p14 |
| P4HA2 | 34390_at | 8974 | −1.17 | −2.25 | 5q31 |
| PBP | 32611_at | 5037 | −0.80 | −1.74 | 12q24.23 |
| PCBD | 34352_at | 5092 | −1.38 | −2.60 | 10q22 |
| PCMT1 | 37736_at | 5110 | −0.63 | −1.55 | 6q24-q25 |
| PEA15 | 32260_at | 8682 | −1.34 | −2.53 | 1q21.1 |
| PHLDA2 | 31888_s_at | 7262 | −0.67 | −1.59 | 11p15.5 |
| PKIG | 34376_at | 11142 | −0.64 | −1.56 | 20q12-q13.1 |
| PODXL | 40434_at | 5420 | −3.94 | −15.39 | 7q32-q33 |
| POLR2L | 35841_at | 5441 | −1.18 | −2.27 | 11p15 |
| POLR2L | 503_at | 5441 | −1.06 | −2.08 | 11p15 |
| PON2 | 40504_at | 5445 | −1.05 | −2.07 | 7q21.3 |
| PPAP2A | 34797_at | 8611 | −1.57 | −2.96 | 5q11 |
| PPP1R3C | 39366_at | 5507 | −1.90 | −3.74 | 10q23-q24 |
| PPP2CB | 924_s_at | 5516 | −0.43 | −1.35 | 8p12-p11.2 |
| PRDX2 | 39729_at | 7001 | −1.86 | −3.64 | 19p13.2 |
| PRSS11 | 718_at | 5654 | −1.33 | −2.51 | 10q26.3 |
| PRSS11 | 719_g_at | 5654 | −1.34 | −2.54 | 10q26.3 |
| PSMD1 | 1314_at | 5707 | −0.65 | −1.57 | 2q37.1 |
| PTGES | 38131_at | 9536 | −1.79 | −3.46 | 9q34.3 |
| PTGIS | 36533_at | 5740 | −3.92 | −15.10 | 20q13.11-q13.13 |
| QKI | 39760_at | 9444 | −1.26 | −2.39 | 6q26-27 |
| RAB4A | 39244_at | 5867 | −3.30 | −9.85 | 1q42-q43 |
| RABIF | 38264_at | 5877 | −1.30 | −2.46 | 1q32-q41 |
| RECK | 35234_at | 8434 | −1.67 | −3.18 | 9p13-p12 |
| RIT1 | 38331_at | 6016 | −1.08 | −2.11 | 1q22 |
| RRAS | 38338_at | 6237 | −0.58 | −1.50 | 19q13.3-qter |
| RRAS2 | 32827_at | 22800 | −1.24 | −2.36 | 11p15.2 |
| S100A10 | 39338_at | 6281 | −1.33 | −2.51 | 1q21 |
| S100A11 | 38138_at | 6282 | −1.07 | −2.09 | 1q21 |
| S100A4 | 38087_s_at | 6275 | −2.38 | −5.20 | 1q21 |
| SDFR1 | 35747_at | 27020 | −0.62 | −1.54 | 15q22 |
| SERPINB2 | 37185_at | 5055 | −1.37 | −2.58 | 18q21.3 |
| SERPINE2 | 41246_at | 5270 | −1.28 | −2.43 | 2q33-q35 |
| SERPING1 | 39775_at | 710 | −1.61 | −3.06 | 11q12-q13.1 |
| SGCD | 34991_at | 6444 | −2.00 | −4.00 | 5q33-q34 |
| SGCD | 34993_at | 6444 | −2.07 | −4.21 | 5q33-q34 |
| SGCD | 41378_at | 6444 | −2.59 | −6.00 | 5q33-q34 |
| SHC1 | 38118_at | 6464 | −0.64 | −1.56 | 1q21 |
| SLC16A4 | 39260_at | 9122 | −3.63 | −12.42 | 1p13.2 |
| SLC20A2 | 36956_at | 6575 | −0.98 | −1.97 | 8p12-q21 |
| SLC4A4 | 35285_at | 8671 | −1.25 | −2.37 | 4q21 |
| SMPD1 | 32574_at | 6609 | −1.23 | −2.35 | 11p15.4-p15.1 |
| SPTAN1 | 33833_at | 6709 | −0.72 | −1.64 | 9q33-q34 |
| STAT1 | 32859_at | 6772 | −1.57 | −2.98 | 2q32.2 |
| STOM | 40419_at | 2040 | −1.88 | −3.69 | 9q34.1 |
| STX12 | 38685_at | 23673 | −1.12 | −2.17 | 1p35-p34.1 |
| STX6 | 41663_at | 10228 | −0.77 | −1.71 | 1q25.1 |
| STX7 | 38774_at | 8417 | −0.84 | −1.79 | 6q23.1 |
| SULF1 | 35832_at | 23213 | −6.48 | −89.21 | 8q13.2 |
| TAGLN | 36931_at | 6876 | −1.78 | −3.43 | 11q23.2 |
| TEK | 1596_g_at | 7010 | −4.15 | −17.71 | 9p21 |
| TM4SF10 | 37958_at | 83604 | −1.32 | −2.49 | Xp11.4 |
| TM7SF1 | 32083_at | 7107 | −2.82 | −7.08 | 1q42-q43 |
| TNFAIP2 | 38631_at | 7127 | −2.45 | −5.47 | 14q32 |
| TNFRSF6 | 1441_s_at | 355 | −2.77 | −6.82 | 10q24.1 |
| TNFRSF6 | 37643_at | 355 | −2.04 | −4.11 | 10q24.1 |
| TPM2 | 32313_at | 7169 | −1.14 | −2.21 | 9p13.2-p13.1 |
| TPM2 | 32314_g_at | 7169 | −0.83 | −1.78 | 9p13.2-p13.1 |
| TRIM22 | 36825_at | 10346 | −4.06 | −16.67 | 11p15 |
| TRIP-Br2 | 37312_at | 9792 | −0.54 | −1.46 | 2p15 |
| TUBB | 39331_at | 7280 | −1.05 | −2.07 | 6p25 |
| UROS | 36652_at | 7390 | −0.71 | −1.64 | 10q25.2-q26.3 |
| VAMP3 | 35783_at | 9341 | −0.47 | −1.39 | 1p36.23 |
| VAMP5 | 32533_s_at | 10791 | −1.71 | −3.27 | 2p11.2 |
| VAT1 | 40147_at | 10493 | −0.77 | −1.70 | 17q21 |
| VEGFC | 159_at | 7424 | −0.99 | −1.98 | 4q34.1-q34.3 |
| VIL2 | 40103_at | 7430 | −1.19 | −2.29 | 6q25.2-q26 |
| ZFHX1B | 35681_r_at | 9839 | −1.11 | −2.16 | 2q22 |
| — | 1586_at | — | −2.26 | −4.78 | — |
| — | 1685_at | — | −1.47 | −2.77 | — |
| — | 1686_g_at | — | −2.97 | −7.81 | — |
| — | 296_at | — | −1.09 | −2.14 | — |
| — | 297_g_at | — | −1.07 | −2.10 | — |
| — | 36867_at | — | −2.03 | −4.09 | — |
| — | 38351_at | — | −4.44 | −21.64 | — |
| — | 39162_at | — | −0.71 | −1.64 | — |
| — | 39170_at | — | −1.35 | −2.54 | — |
| — | 39750_at | — | −1.46 | −2.76 | — |
| — | 631_g_at | — | −0.88 | −1.84 | — |
| — | AFFX-BioC-3_at | — | −2.22 | −4.67 | — |
| — | AFFX-CreX-5_at | — | −3.49 | −11.26 | — |

Table 13. Summary of differentially regulated genes/probes after demethylation in immortal MDAH041 and MDAH087 cells.

TABLE 13a
49 probes downregulated 5-aza-dC vs IM (see Table 1C)

| | Affymetrix | | Average | Fold | |
|---|---|---|---|---|---|
| Symbol | HGU95Av2 Probe ID | LocusLink | Signal Log₂ | Change | Locus |
| AARS | 36185_at | 16 | −0.87 | −1.83 | 16q22 |
| AD-017 | 33126_at | 55830 | −0.63 | −1.55 | 3p21.31 |
| ARHA | 37309_at | 387 | −0.42 | −1.34 | 3p21.3 |
| ARHGEF6 | 37543_at | 9459 | −0.91 | −1.88 | Xq26 |
| AXL | 38433_at | 558 | −0.75 | −1.68 | 19q13.1 |
| C5orf13 | 39710_at | 9315 | −1.56 | −2.94 | 5q22.2 |
| CCNF | 35907_at | 899 | −1.04 | −2.06 | 16p13.3 |
| CDC25B | 1347_at | 994 | −0.98 | −1.97 | 20p13 |
| CDKN2C | 36053_at | 1031 | −1.27 | −2.41 | 1p32 |
| CPN2 | 34778_at | 1370 | −0.91 | −1.88 | 8p23-p22 |
| DHCR24 | 36658_at | 1718 | −0.68 | −1.60 | 1p33-p31.1 |
| EVI2A | 36313_at | 2123 | −1.20 | −2.29 | 17q11.2 |
| F2R | 41700_at | 2149 | −0.79 | −1.73 | 5q13 |
| GBE1 | 32643_at | 2632 | −0.85 | −1.80 | 3p12.3 |
| GCN1L1 | 36603_at | 10985 | −0.56 | −1.47 | 12q24.2 |
| HRMT1L1 | 39348_at | 3275 | −0.73 | −1.65 | 21q22.3 |
| ICT1 | 40758_at | 3396 | −0.87 | −1.82 | 17q25.2 |
| INSIG1 | 35303_at | 3638 | −1.15 | −2.21 | 7q36 |
| KIAA0372 | 40517_at | 9652 | −0.91 | −1.88 | 5q15 |
| KIAA1049 | 41268_g_at | 22980 | −0.87 | −1.82 | 16q24.3 |
| LDB2 | 36065_at | 9079 | −1.30 | −2.47 | 4p16 |
| MAP1A | 35917_at | 4130 | −1.12 | −2.17 | 15q13-qter |
| MRPL23 | 34358_at | 6150 | −0.78 | −1.72 | 11p15.5-p15.4 |
| MRPL9 | 41514_s_at | 65005 | −0.61 | −1.52 | 1 |
| MYST4 | 35203_at | 23522 | −0.73 | −1.65 | 10q22.2 |
| NAG | 31896_at | 51594 | −0.72 | −1.64 | 2p24 |
| NDUFS4 | 38695_at | 4724 | −0.70 | −1.62 | 5q11.1 |
| PABPC4 | 40506_s_at | 8761 | −0.60 | −1.51 | 1p32-p36 |
| PDGFRA | 1731_at | 5156 | −1.49 | −2.81 | 4q11-q13 |
| PGAM1 | 41221_at | 5223 | −0.59 | −1.51 | 10q25.3 |
| POLD2 | 1470_at | 5425 | −0.80 | −1.74 | 7p13 |
| PPP2R5C | 40784_at | 5527 | −0.75 | −1.68 | 14q32 |
| RAD23B | 1874_at | 5887 | −0.68 | −1.60 | 9q31.2 |
| RECK | 35234_at | 8434 | −1.93 | −3.81 | 9p13-p12 |
| RECK | 35236_g_at | 8434 | −1.32 | −2.49 | 9p13-p12 |
| RME8 | 39403_at | 23317 | −0.82 | −1.76 | 3q22.1 |
| RNASE4 | 32664_at | 6038 | −1.44 | −2.71 | 14q11.1 |
| SLC35E2 | 41243_at | 9906 | −0.70 | −1.63 | 1p36.33 |
| SMARCD3 | 32565_at | 6604 | −1.24 | −2.37 | 7q35-q36 |
| TCFL5 | 35614_at | 10732 | −0.98 | −1.98 | 20q13.3-qter |
| TM4SF10 | 37958_at | 83604 | −1.29 | −2.45 | Xp11.4 |
| TUBGCP3 | 38353_at | 10426 | −1.05 | −2.06 | 13q34 |
| WDR18 | 35983_at | 57418 | −0.67 | −1.59 | 19p13.3 |
| — | 33451_s_at | — | −0.88 | −1.84 | — |
| — | 34283_at | — | −1.89 | −3.70 | — |
| — | 34367_at | — | −0.82 | −1.77 | — |
| — | 38440_s_at | — | −0.73 | −1.65 | — |
| — | 39750_at | — | −0.94 | −1.92 | — |
| — | 40567_at | — | −0.84 | −1.79 | — |

TABLE 13b
226 probes upregulated 5-aza-dC vs IM (see Table 1D)

| | | | | | |
|---|---|---|---|---|---|
| ADFP | 34378_at | 123 | 1.06 | 2.09 | 9p22.1 |
| AKR1B1 | 36589_at | 231 | 0.66 | 1.58 | 7q35 |
| ALDH1A3 | 36686_at | 220 | 1.58 | 2.99 | 15q26.3 |
| ATF3 | 287_at | 467 | 1.40 | 2.64 | 1q32.3 |
| ATF5 | 39158_at | 22809 | 2.24 | 4.71 | 19q13.3 |
| ATP2B1 | 37661_at | 490 | 0.76 | 1.69 | 12q21-q23 |
| ATP6V1H | 33741_at | 51606 | 0.58 | 1.50 | 8p22-q22.3 |
| BAZ1A | 37971_at | 11177 | 0.68 | 1.61 | 14q12-q13 |
| BCAP31 | 41724_at | 10134 | 0.62 | 1.54 | Xq28 |
| BHLHB2 | 40790_at | 8553 | 0.85 | 1.81 | 3p26 |
| BIRC3 | 1717_s_at | 330 | 2.97 | 7.81 | 11q22 |
| BTG3 | 37218_at | 10950 | 0.81 | 1.75 | 21q21.1-q21.2 |
| C20orf18 | 32203_at | 10616 | 0.76 | 1.70 | 20p13 |
| C6orf9 | 39049_at | 63940 | 1.73 | 3.32 | 6p21.3 |
| C20orf18 | 32203_at | 10616 | 0.76 | 1.70 | 20p13 |
| C6orf9 | 39049_at | 63940 | 1.73 | 3.32 | 6p21.3 |
| CASP7 | 38281_at | 840 | 0.77 | 1.70 | 10q25 |
| CASP8 | 33774_at | 841 | 0.95 | 1.94 | 2q33-q34 |
| CCL20 | 40385_at | 6364 | 6.10 | 68.57 | 2q33-q37 |
| CDC34 | 1274_s_at | 997 | 0.89 | 1.85 | 19p13.3 |
| CLTB | 32523_at | 1212 | 0.94 | 1.92 | 4q2-q3 |
| CLU | 36780_at | 1191 | 1.15 | 2.21 | 8p21-p12 |
| CRADD | 1211_s_at | 8738 | 1.66 | 3.16 | 12q21.33-q23.1 |
| CREG | 35311_at | 8804 | 1.29 | 2.45 | 1q24 |
| CREM | 32067_at | 1390 | 0.81 | 1.75 | 10p11.21 |
| CTAG1 | 33636_at | 1485 | 4.23 | 18.72 | Xq28 |
| CTAG1 | 33637_g_at | 1485 | 2.65 | 6.29 | Xq28 |
| CXCL2 | 37187_at | 2920 | 3.22 | 9.33 | 4q21 |
| CXCL3 | 34022_at | 2921 | 3.37 | 10.36 | 4q21 |
| CXCL6 | 35410_at | 6372 | 2.36 | 5.12 | 4q21 |
| CYP1B1 | 859_at | 1545 | 2.64 | 6.22 | 2p21 |
| CYP1B1 | 40071_at | 1545 | 2.29 | 4.88 | 2p21 |
| D6S2654E | 34957_at | 26240 | 1.60 | 3.02 | 6p25-pter |
| DAZL | 33972_r_at | 1618 | 5.90 | 59.74 | 3p24.3 |
| DAZL | 33971_f_at | 1618 | 5.84 | 57.19 | 3p24.3 |
| DDR1 | 36643_at | 780 | 0.89 | 1.86 | 6p21.3 |
| DKFZP564M182 | 37161_at | 26156 | 0.91 | 1.88 | 16p13.13 |
| DNAJA1 | 39118_at | 3301 | 0.87 | 1.83 | 9p13-p12 |
| DNAJA1 | 276_at | 3301 | 0.78 | 1.72 | 9p13-p12 |
| DOCK4 | 41620_at | 9732 | 1.11 | 2.16 | 7q31.1 |
| DUSP1 | 1005_at | 1843 | 1.22 | 2.33 | 5q34 |
| DUSP11 | 39727_at | 8446 | 0.33 | 1.26 | 2p13.1 |
| DUSP5 | 529_at | 1847 | 1.65 | 3.14 | 10q25 |
| DUSP6 | 41193_at | 1848 | 1.82 | 3.54 | 12q22-q23 |
| EEF1A2 | 35174_i_at | 1917 | 1.62 | 3.08 | 20q13.3 |
| ELL2 | 40606_at | 22936 | 0.79 | 1.72 | 5q15 |
| ETV6 | 38491_at | 2120 | 0.79 | 1.73 | 12p13 |
| F2RL1 | 38247_at | 2150 | 2.89 | 7.43 | 5q13 |
| F3 | 36543_at | 2152 | 1.08 | 2.12 | 1p22-p21 |
| FGF2 | 1828_s_at | 2247 | 0.90 | 1.87 | 4q26-q27 |
| FLJ10097 | 40916_at | 56271 | 0.89 | 1.85 | Xq22.1-q22 |
| FLJ14675 | 41207_at | 84909 | 1.20 | 2.30 | 9q22.33 |
| FLJ23027 | 33915_at | 84193 | 0.49 | 1.41 | 14q32.32 |
| FMR1 | 37994_at | 2332 | 0.62 | 1.53 | Xq27.3 |
| FUS | 39420_at | 2521 | 1.39 | 2.62 | 16p11.2 |
| G0S2 | 38326_at | 50486 | 2.61 | 6.11 | 1q32.2-q41 |
| G1P2 | 1107_s_at | 9636 | 2.26 | 4.80 | 1p36.33 |
| GAGE1 | 31497_at | 2543 | 1.92 | 3.79 | Xp11.4-p11 |
| GAGE5 | 31954_f_at | 2577 | 6.16 | 71.51 | Xp11.4-p11 |
| GAGE5 | 33671_f_at | 2577 | 6.15 | 70.77 | Xp11.4-p11 |
| GAGE5 | 31960_f_at | 2577 | 5.93 | 60.89 | Xp11.4-p11 |
| GAGE5 | 37065_f_at | 2577 | 5.67 | 50.77 | Xp11.4-p11 |
| GAGE5 | 33680_f_at | 2577 | 5.64 | 49.89 | Xp11.4-p11 |
| GAGE5 | 31498_f_at | 2577 | 5.53 | 46.10 | Xp11.4-p11 |
| GALE | 31598_s_at | 2582 | 1.54 | 2.91 | 1p36-p35 |
| GCH1 | 37944_at | 2643 | 2.94 | 7.68 | 14q22.1-q22.2 |
| GCLC | 31850_at | 2729 | 0.89 | 1.86 | 6p12 |
| GFPT1 | 32626_at | 2673 | 0.61 | 1.53 | 2p13 |
| GLRX | 34311_at | 2745 | 1.43 | 2.69 | 5q14 |
| HCLS1 | 31820_at | 3059 | 2.80 | 6.94 | 3q13 |
| HDAC9 | 37483_at | 9734 | 1.52 | 2.86 | 7p21.1 |
| HES1 | 37393_at | 3280 | 1.91 | 3.77 | 3q28-q29 |
| HIST1H1C | 37018_at | 3006 | 1.84 | 3.58 | 6p21.3 |
| HIST1H2AC | 34308_at | 8334 | 1.14 | 2.20 | 6p21.3 |
| HIST1H2AG | 284_at | 8969 | 2.84 | 7.17 | 6p22.1 |
| HIST1H2AG | 285_g_at | 8969 | 2.35 | 5.09 | 6p22.1 |
| HIST1H2BK | 32819_at | 85236 | 1.05 | 2.07 | 6p21.33 |
| HIST1H2BL | 35576_f_at | 8340 | 1.65 | 3.13 | 6p22-p21.3 |
| HIST1H2BN | 36347_f_at | 8341 | 1.75 | 3.37 | 6p22-p21.3 |
| HIST1H3D | 34964_at | 8351 | 2.95 | 7.73 | 6p21.3 |
| HIST2H2AA | 32609_at | 8337 | 2.76 | 6.78 | 1q21.3 |
| HIST2H2AA | 286_at | 8337 | 2.45 | 5.48 | 1q21.3 |
| HIST2H2BE | 33352_at | 8349 | 1.84 | 3.57 | 1q21-q23 |
| HLA-C | 37383_f_at | 3107 | 1.36 | 2.56 | 6p21.3 |
| HPS5 | 35223_at | 11234 | 0.80 | 1.74 | 11p14 |
| HSPA2 | 36925_at | 3306 | 2.30 | 4.92 | 14q24.1 |
| HTATIP2 | 38824_at | 10553 | 1.68 | 3.19 | 11p15.1 |
| ICAM1 | 32640_at | 3383 | 4.15 | 17.76 | 19p13.3-p13.2 |
| IER3 | 1237_at | 8870 | 0.91 | 1.88 | 6p21.3 |
| IFIT4 | 38584_at | 3437 | 2.79 | 6.93 | 10q24 |
| IFRD1 | 32901_s_at | 3475 | 0.93 | 1.91 | 7q22-q31 |
| IGFBP7 | 2062_at | 3490 | 1.16 | 2.23 | 4q12 |
| IL11 | 35464_at | 3589 | 1.44 | 2.71 | 19q13.3-q13.4 |
| IL13RA2 | 1016_s_at | 3598 | 3.31 | 9.94 | Xq13.1-q28 |
| IL1B | 39402_at | 3553 | 2.95 | 7.75 | 2q14 |
| IL6 | 38299_at | 3569 | 3.66 | 12.63 | 7p21 |
| IL8 | 35372_r_at | 3576 | 3.20 | 9.20 | 4q13-q21 |
| INHBA | 40357_at | 3624 | 1.08 | 2.11 | 7p15-p13 |
| INPP5F | 36089_at | 22876 | 1.52 | 2.86 | 10q26.13 |
| ISG20 | 33304_at | 3669 | 2.08 | 4.24 | 15q26 |
| ITGA2 | 41481_at | 3673 | 2.89 | 7.43 | 5q23-q31 |
| KIAA0247 | 38393_at | 9766 | 1.03 | 2.04 | 14q24.1 |
| KIAA0690 | 36520_at | 23223 | 1.38 | 2.61 | 10q24.2 |
| KIAA1111 | 41399_at | 23133 | 0.82 | 1.76 | Xp11.22 |
| KIAA1750 | 32730_at | 85453 | 2.19 | 4.58 | 8q22.1 |
| KRT18 | 35766_at | 3875 | 3.28 | 9.69 | 12q13 |
| KRT7 | 41294_at | 3855 | 2.37 | 5.17 | 12q12-q13 |
| KRTHB1 | 36288_at | 3887 | 5.53 | 46.25 | 12q13 |
| LAMB3 | 36929_at | 3914 | 1.95 | 3.86 | 1q32 |
| LAMP2 | 38403_at | 3920 | 0.64 | 1.56 | Xq24 |
| LGALS3BP | 37754_at | 3959 | 2.66 | 6.31 | 17q25 |
| LOC56902 | 33720_at | 56902 | 0.73 | 1.66 | 2p13.3 |
| LPXN | 36062_at | 9404 | 0.88 | 1.84 | 11q12.1 |
| LRIG1 | 34800_at | 26018 | 1.25 | 2.38 | 3p14 |
| LRRN4 | 37796_at | 4034 | 1.42 | 2.67 | 7q22 |
| MADH3 | 1454_at | 4088 | 0.73 | 1.66 | 15q21-q22 |
| MAFF | 36711_at | 23764 | 1.93 | 3.82 | 22q13.1 |
| MAGEA2 | 33518_f_at | 4101 | 3.45 | 10.95 | Xq28 |
| MAGEA4 | 36302_f_at | 4103 | 4.68 | 25.58 | Xq28 |
| MAGEA5 | 34575_f_at | 4104 | 2.85 | 7.21 | Xq28 |
| MAGEB2 | 35097_at | 4113 | 5.26 | 38.20 | Xp21.3 |
| MAGEC1 | 34932_at | 9947 | 2.68 | 6.42 | Xq26 |
| MAP1LC3B | 39370_at | 81631 | 1.14 | 2.20 | 16q24.2 |
| MAP2K3 | 2075_s_at | 5606 | 0.60 | 1.51 | 17q11.2 |
| MAX | 1981_s_at | 4149 | 0.93 | 1.91 | 14q23 |
| MICB | 35937_at | 4277 | 0.91 | 1.88 | 6p21.3 |
| MLLT2 | 39037_at | 4299 | 0.67 | 1.59 | 4q21 |
| MMP1 | 38428_at | 4312 | 2.56 | 5.90 | 11q22.3 |
| MMP13 | 39632_at | 4322 | 4.05 | 16.52 | 11q22.3 |
| MPG | 37768_at | 4350 | 0.75 | 1.69 | 16p13.3 |
| MYD88 | 38369_at | 4615 | 0.96 | 1.95 | 3p22 |
| NAB1 | 38692_at | 4664 | 0.91 | 1.88 | 2q32.3-q33 |
| NFKB1 | 1377_at | 4790 | 0.92 | 1.89 | 4q24 |
| NFKB1 | 1378_g_at | 4790 | 0.87 | 1.83 | 4q24 |
| NFKB1 | 38438_at | 4790 | 0.61 | 1.53 | 4q24 |
| NFKB2 | 544_at | 4791 | 1.44 | 2.70 | 10q24 |
| NFKB2 | 40362_at | 4791 | 1.29 | 2.44 | 10q24 |
| NFKBIA | 1461_at | 4792 | 1.02 | 2.02 | 14q13 |
| NFKBIE | 38276_at | 4794 | 1.29 | 2.44 | 6p21.1 |
| NMB | 40686_at | 4828 | 1.47 | 2.78 | 15q22-qter |
| NMI | 36472_at | 9111 | 0.92 | 1.90 | 2p24.3-q21 |
| NOL1 | 1979_s_at | 4839 | 0.76 | 1.69 | 12p13 |
| NXT2 | 35136_at | 55916 | 1.48 | 2.79 | Xq23 |
| OPTN | 41742_s_at | 10133 | 0.81 | 1.75 | 10p14 |
| PALM2 | 35985_at | 114299 | 0.61 | 1.53 | 9q31-q33 |
| PBEF | 33849_at | 10135 | 1.89 | 3.70 | 7q22.2 |
| PDAP2 | 38115_at | 11334 | 0.52 | 1.43 | 3p21.3 |
| PISD | 38090_at | 23761 | 0.72 | 1.64 | 22q12.2 |
| PLAB | 1890_at | 9518 | 1.96 | 3.88 | 19p13.1-13 |
| PLAT | 33452_at | 5327 | 1.15 | 2.23 | 8p12 |
| PLAU | 37310_at | 5328 | 2.28 | 4.87 | 10q24 |
| PLEKHB2 | 39525_at | 55041 | 1.14 | 2.21 | 2q21.2 |
| PMAIP1 | 41048_at | 5366 | 1.89 | 3.72 | 18q21.32 |
| PSCD1 | 38666_at | 9267 | 1.03 | 2.04 | 17q25 |
| PSMB8 | 41184_s_at | 5696 | 1.02 | 2.02 | 6p21.3 |
| PTPRR | 1658_g_at | 5801 | 0.84 | 1.79 | 12q15 |
| PVRL2 | 32156_at | 5819 | 0.77 | 1.71 | 19q13.2-q13.4 |
| RAB9A | 39628_at | 9367 | 0.52 | 1.44 | Xp22.2 |
| RBBP4 | 1318_at | 5928 | 0.83 | 1.77 | 1p34.3 |
| RBP1 | 38634_at | 5947 | 2.33 | 5.02 | 3q23 |
| RELB | 570_at | 5971 | 1.40 | 2.65 | 19q13.32 |
| RNF44 | 41179_at | 22838 | 1.02 | 2.02 | 5q35.3 |
| RRAD | 1776_at | 6236 | 3.81 | 14.05 | 16q22 |
| RRAD | 39528_at | 6236 | 3.50 | 11.31 | 16q22 |
| S100A2 | 2027_at | 6273 | 0.87 | 1.82 | 1q21 |
| SAA1 | 33272_at | 6288 | 2.74 | 6.66 | 11p15.1 |
| SAT | 34304_s_at | 6303 | 1.10 | 2.14 | Xp22.1 |
| SATB2 | 41708_at | 23314 | 0.58 | 1.50 | 2q33 |
| SCG2 | 36924_r_at | 7857 | 1.55 | 2.93 | 2q35-q36 |
| SERPINB2 | 37185_at | 5055 | 2.85 | 7.23 | 18q21.3 |
| SERPINB8 | 36312_at | 5271 | 1.23 | 2.34 | 18q21.3 |
| SFN | 33323_r_at | 2810 | 1.52 | 2.87 | 1p35.3 |
| SFN | 33322_i_at | 2810 | 1.26 | 2.39 | 1p35.3 |
| SLC39A8 | 40456_at | 64116 | 1.12 | 2.18 | 4q22-q24 |
| SMOX | 1649_at | 54498 | 1.08 | 2.12 | 20p13 |
| SMOX | 1650_g_at | 54498 | 1.06 | 2.09 | 20p13 |
| SNAPC1 | 35488_at | 6617 | 1.50 | 2.82 | 14q22 |
| SOD2 | 34666_at | 6648 | 1.53 | 2.90 | 6q25.3 |
| SQSTM1 | 40898_at | 8878 | 1.16 | 2.23 | 5q35 |
| SSX2 | 36409_f_at | 6757 | 5.63 | 49.57 | Xp11.23-p11.22 |
| SSX3 | 33655_f_at | 10214 | 2.61 | 6.11 | Xp11.23 |
| SSX4 | 35950_at | 6759 | 2.20 | 4.60 | Xp11.23 |
| STC1 | 41354_at | 6781 | 1.13 | 2.19 | 8p21-p11.2 |
| TAP1 | 40153_at | 6890 | 1.48 | 2.79 | 6p21.3 |
| TAX1BP1 | 35279_at | 8887 | 0.48 | 1.39 | 7p15 |
| TERF2IP | 38982_at | 54386 | 1.05 | 2.07 | 16q23.1 |
| TES | 32134_at | 26136 | 1.76 | 3.39 | 7q31.2 |
| TFPI2 | 37388_at | 7980 | 3.99 | 15.85 | 7q22 |
| TGM2 | 38404_at | 7052 | 3.93 | 15.24 | 20q12 |
| TGM2 | 231_at | 7052 | 2.66 | 6.34 | 20q12 |
| TKTL1 | 37120_at | 8277 | 6.35 | 81.66 | Xq28 |
| TNFAIP2 | 38631_at | 7127 | 1.90 | 3.73 | 14q32 |
| TNFAIP3 | 595_at | 7128 | 1.49 | 2.81 | 6q23 |
| TNFRSF10B | 34892_at | 8795 | 1.09 | 2.13 | 8p22-p21 |
| TNIP1 | 38970_s_at | 10318 | 0.84 | 1.79 | 5q32-q33.1 |
| TOP1 | 1710_s_at | 7150 | 0.83 | 1.78 | 20q12-q13. |
| TRAF1 | 849_g_at | 7185 | 1.69 | 3.22 | 9q33-q34 |
| TSNAX | 41051_at | 7257 | 0.52 | 1.44 | 1q42.1 |
| TXNRD1 | 39425_at | 7296 | 0.59 | 1.50 | 12q23-q24. |
| UPP1 | 37351_at | 7378 | 1.70 | 3.25 | 7p12.3 |
| WBSCR22 | 40090_at | 114049 | 1.07 | 2.10 | |
| ZNF267 | 34544_at | 10308 | 1.05 | 2.07 | 16p11.2 |
| — | 1173_g_at | — | 1.19 | 2.28 | — |
| — | 126_s_at | — | 5.19 | 36.52 | — |
| — | 1369_s_at | — | 3.84 | 14.33 | — |
| — | 1520_s_at | — | 3.72 | 13.16 | — |
| — | 153_f_at | — | 1.91 | 3.75 | — |
| — | 1693_s_at | — | 0.83 | 1.78 | — |
| — | 1842_at | — | 1.42 | 2.67 | — |
| — | 189_s_at | — | 1.18 | 2.27 | — |
| — | 291_s_at | — | 3.14 | 8.79 | — |
| — | 31480_f_at | — | 3.22 | 9.30 | — |
| — | 31522_f_at | — | 1.68 | 3.21 | — |
| — | 31523_f_at | — | 1.34 | 2.53 | — |
| — | 31524_f_at | — | 1.35 | 2.55 | — |
| — | 31528_f_at | — | 1.12 | 2.17 | — |
| — | 31633_g_at | — | 0.71 | 1.64 | — |
| — | 31953_f_at | — | 3.11 | 8.66 | — |
| — | 32426_f_at | — | 3.54 | 11.61 | — |
| — | 32980_f_at | — | 1.41 | 2.65 | — |
| — | 330_s_at | — | 1.42 | 2.68 | — |
| — | 33761_s_at | 376645 | 0.85 | 1.80 | 1q21.2 |
| — | 34577_at | — | 1.43 | 2.70 | — |
| — | 35994_at | — | 0.77 | 1.70 | — |
| — | 36757_at | — | 2.58 | 5.98 | — |
| — | 408_at | — | 4.31 | 19.85 | — |
| — | 645_at | — | 3.70 | 12.98 | — |
| — | 669_s_at | — | 1.07 | 2.10 | — |

TABLE 14

| | | | Immortal | | | | | | 5-aza-dC | | | | | |
|---|---|---|---|---|---|---|---|---|---|---|---|---|---|---|
| Term | GO ID | Total | Down | P | P* | Up | P | P* | Down | P | P* | Up | P | P* |

A. Biological process

| | | | | | | | | | | | | | | |
|---|---|---|---|---|---|---|---|---|---|---|---|---|---|---|
| Cell adhesion | 0007155 | 361 | 13 | 0.03 | 1.00 | 4 | 0.93 | 1.00 | 0 | 1.00 | 1.00 | 7 | 0.74 | 1.00 |
| cell—cell adhesion | 0016337 | 124 | 3 | 0.46 | 1.00 | 2 | 0.71 | 1.00 | 0 | 1.00 | 1.00 | 1 | 0.95 | 1.00 |
| Cell-cell signaling | 0007267 | 365 | 6 | 0.75 | 1.00 | 4 | 0.94 | 1.00 | 0 | 1.00 | 1.00 | 11 | 0.23 | 1.00 |
| Signal transduction | 0007165 | 1321 | 25 | 0.67 | 1.00 | 25 | 0.63 | 1.00 | 6 | 0.56 | 1.00 | 33 | 0.35 | 1.00 |
| cell surface receptor linked | 0007166 | 588 | 11 | 0.65 | 1.00 | 17 | 0.07 | 1.00 | 2 | 0.76 | 1.00 | 14 | 0.51 | 1.00 |
| signal transduction | | | | | | | | | | | | | | |
| intracellular signaling cascade | 0007242 | 444 | 7 | 0.80 | 1.00 | 8 | 0.66 | 1.00 | 2 | 0.60 | 1.00 | 10 | 0.59 | 1.00 |
| Cell death | 0008219 | 272 | 6 | 0.47 | 1.00 | 9 | 0.09 | 1.00 | 2 | 0.35 | 1.00 | 15 | 0.00 | 1.00 |
| anti-apoptosis | 0006916 | 59 | 3 | 0.11 | 1.00 | 4 | 0.03 | 1.00 | 0 | 1.00 | 1.00 | 5 | 0.01 | 1.00 |
| apoptotic program | 0008632 | 26 | 1 | 0.41 | 1.00 | 1 | 0.41 | 1.00 | 1 | 0.11 | 1.00 | 3 | 0.02 | 1.00 |
| induction of apoptosis | 0006917 | 83 | 2 | 0.50 | 1.00 | 3 | 0.23 | 1.00 | 0 | 1.00 | 1.00 | 3 | 0.30 | 1.00 |
| induction of programmed cell | 0012502 | 83 | 2 | 0.50 | 1.00 | 3 | 0.23 | 1.00 | 0 | 1.00 | 1.00 | 3 | 0.30 | 1.00 |
| death | | | | | | | | | | | | | | |
| Cell differentiation | 0030154 | 106 | 2 | 0.63 | 1.00 | 2 | 0.62 | 1.00 | 1 | 0.38 | 1.00 | 5 | 0.10 | 1.00 |
| lymphocyte differentiation | 0030098 | 22 | 0 | 1.00 | 1.00 | 0 | 1.00 | 1.00 | 0 | 1.00 | 1.00 | 3 | 0.01 | 1.00 |
| Cell organization and | 0016043 | 282 | 5 | 0.68 | 1.00 | 10 | 0.05 | 1.00 | 0 | 1.00 | 1.00 | 12 | 0.03 | 1.00 |
| biogenesis | | | | | | | | | | | | | | |
| cytoplasm organization and | 0007028 | 166 | 5 | 0.24 | 1.00 | 4 | 0.42 | 1.00 | 0 | 1.00 | 1.00 | 1 | 0.98 | 1.00 |
| biogenesis | | | | | | | | | | | | | | |
| nuclear organization and | 0006997 | 109 | 0 | 1.00 | 1.00 | 6 | 0.02 | 1.00 | 0 | 1.00 | 1.00 | 11 | 0.00 | 1.00 |
| biogenesis | | | | | | | | | | | | | | |
| Cell proliferation | 0008283 | 667 | 18 | 0.12 | 1.00 | 37 | 0.00 | 0.00 | 11 | 0.00 | 1.00 | 18 | 0.29 | 1.00 |
| cell cycle | 0007049 | 442 | 12 | 0.18 | 1.00 | 32 | 0.00 | 0.00 | 7 | 0.00 | 1.00 | 14 | 0.15 | 1.00 |
| regulation of cell proliferation | 0042127 | 175 | 8 | 0.02 | 1.00 | 7 | 0.06 | 1.00 | 3 | 0.04 | 1.00 | 8 | 0.05 | 1.00 |
| cytokinesis | 0000910 | 64 | 1 | 0.73 | 1.00 | 5 | 0.01 | 0.57 | 2 | 0.03 | 1.00 | 0 | 1.00 | 1.00 |
| Transport | 0006810 | 873 | 18 | 0.49 | 1.00 | 13 | 0.90 | 1.00 | 1 | 0.99 | 1.00 | 12 | 0.99 | 1.00 |
| intracellular transport | 0046907 | 258 | 6 | 0.42 | 1.00 | 3 | 0.89 | 1.00 | 1 | 0.69 | 1.00 | 6 | 0.56 | 1.00 |
| ion transport | 0006811 | 320 | 7 | 0.47 | 1.00 | 7 | 0.45 | 1.00 | 0 | 1.00 | 1.00 | 2 | 1.00 | 1.00 |
| protein transport | 0015031 | 223 | 5 | 0.47 | 1.00 | 2 | 0.94 | 1.00 | 1 | 0.64 | 1.00 | 7 | 0.26 | 1.00 |
| vesicle-mediated transport | 0016192 | 167 | 7 | 0.05 | 1.00 | 3 | 0.65 | 1.00 | 0 | 1.00 | 1.00 | 2 | 0.91 | 1.00 |
| Cell motility | 0006928 | 222 | 10 | 0.01 | 1.00 | 3 | 0.82 | 1.00 | 1 | 0.64 | 1.00 | 1 | 1.00 | 1.00 |
| cell migration | 0016477 | 39 | 5 | 0.00 | 1.00 | 1 | 0.54 | 1.00 | 0 | 1.00 | 1.00 | 1 | 0.60 | 1.00 |
| Metabolism | 0008152 | 3192 | 44 | 1.00 | 1.00 | 78 | 0.00 | 1.00 | 18 | 0.11 | 1.00 | 78 | 0.29 | 1.00 |
| alcohol metabolism | 0006066 | 151 | 2 | 0.81 | 1.00 | 4 | 0.35 | 1.00 | 2 | 0.15 | 1.00 | 4 | 0.47 | 1.00 |
| amine metabolism | 0009308 | 175 | 2 | 0.87 | 1.00 | 6 | 0.13 | 1.00 | 1 | 0.55 | 1.00 | 3 | 0.78 | 1.00 |
| amino acid and derivative | 0006519 | 149 | 2 | 0.81 | 1.00 | 4 | 0.34 | 1.00 | 1 | 0.49 | 1.00 | 3 | 0.68 | 1.00 |
| metabolism | | | | | | | | | | | | | | |
| biosynthesis | 0009058 | 525 | 7 | 0.91 | 1.00 | 9 | 0.72 | 1.00 | 6 | 0.03 | 1.00 | 7 | 0.97 | 1.00 |
| carbohydrate metabolism | 0005975 | 228 | 3 | 0.84 | 1.00 | 6 | 0.30 | 1.00 | 2 | 0.27 | 1.00 | 4 | 0.78 | 1.00 |
| catabolism | 0009056 | 450 | 4 | 0.98 | 1.00 | 8 | 0.67 | 1.00 | 2 | 0.61 | 1.00 | 9 | 0.73 | 1.00 |
| electron transport | 0006118 | 150 | 6 | 0.08 | 1.00 | 4 | 0.34 | 1.00 | 2 | 0.14 | 1.00 | 4 | 0.46 | 1.00 |
| energy pathways | 0006091 | 158 | 3 | 0.62 | 1.00 | 4 | 0.38 | 1.00 | 2 | 0.16 | 1.00 | 1 | 0.98 | 1.00 |
| lipid metabolism | 0006629 | 299 | 6 | 0.56 | 1.00 | 5 | 0.71 | 1.00 | 1 | 0.75 | 1.00 | 4 | 0.92 | 1.00 |
| nucleobase, nucleoside, | 0006139 | 1351 | 12 | 1.00 | 1.00 | 41 | 0.00 | 1.00 | 6 | 0.59 | 1.00 | 41 | 0.03 | 1.00 |
| nucleotide and nucleic acid | | | | | | | | | | | | | | |
| metabolism | | | | | | | | | | | | | | |
| organic acid metabolism | 0006082 | 226 | 5 | 0.48 | 1.00 | 5 | 0.46 | 1.00 | 1 | 0.64 | 1.00 | 3 | 0.90 | 1.00 |
| phosphorus metabolism | 0006793 | 423 | 6 | 0.86 | 1.00 | 17 | 0.00 | 1.00 | 5 | 0.04 | 1.00 | 9 | 0.66 | 1.00 |
| protein metabolism | 0019538 | 1170 | 16 | 0.97 | 1.00 | 25 | 0.37 | 1.00 | 9 | 0.06 | 1.00 | 27 | 0.55 | 1.00 |
| regulation of metabolism | 0019222 | 32 | 2 | 0.13 | 1.00 | 0 | 1.00 | 1.00 | 0 | 1.00 | 1.00 | 1 | 0.53 | 1.00 |
| Response to endogenous | 0009719 | 134 | 1 | 0.94 | 1.00 | 4 | 0.27 | 1.00 | 1 | 0.46 | 1.00 | 2 | 0.82 | 1.00 |
| stimulus | | | | | | | | | | | | | | |
| response to DNA damage | 0006974 | 133 | 1 | 0.93 | 1.00 | 4 | 0.27 | 1.00 | 1 | 0.45 | 1.00 | 2 | 0.82 | 1.00 |
| stimulus | | | | | | | | | | | | | | |
| response to external stimulus | 0009605 | 813 | 18 | 0.37 | 1.00 | 6 | 1.00 | 1.00 | 2 | 0.90 | 1.00 | 30 | 0.01 | 1.00 |
| response to abiotic stimulus | 0009628 | 224 | 2 | 0.94 | 1.90 | 4 | 0.65 | 1.00 | 0 | 1.00 | 1.00 | 8 | 0.15 | 1.00 |
| response to biotic stimulus | 0009607 | 552 | 13 | 0.32 | 1.00 | 1 | 1.00 | 1.00 | 1 | 0.93 | 1.00 | 26 | 0.00 | 1.00 |
| Response to extracellular | 0009991 | 10 | 0 | 1.00 | 1.00 | 0 | 1.00 | 1.00 | 0 | 1.00 | 1.00 | 1 | 0.21 | 1.00 |
| stimulus | | | | | | | | | | | | | | |
| response to wounding | 0009611 | 173 | 3 | 0.68 | 1.00 | 1 | 0.97 | 1.00 | 1 | 0.55 | 1.00 | 14 | 0.00 | 0.00 |
| taxis | 0042330 | 77 | 1 | 0.79 | 1.00 | 1 | 0.79 | 1.00 | 0 | 1.00 | 1.00 | 7 | 0.00 | 1.00 |
| Response to stress | 0006950 | 501 | 12 | 0.31 | 1.00 | 7 | 0.88 | 1.00 | 3 | 0.39 | 1.00 | 21 | 0.01 | 1.00 |
| response to DNA damage | 0006974 | 133 | 1 | 0.93 | 1.00 | 4 | 0.27 | 1.00 | 1 | 0.45 | 1.00 | 2 | 0.82 | 1.00 |
| stimulus | | | | | | | | | | | | | | |
| response to | 0009613 | 287 | 8 | 0.22 | 1.00 | 1 | 1.00 | 1.00 | 1 | 0.73 | 1.00 | 17 | 0.00 | 1.00 |
| pest/pathogen/parasite | | | | | | | | | | | | | | |
| Bbiological—process unknown | 0000004 | 232 | 3 | 0.85 | 1.00 | 4 | 0.68 | 1.00 | 2 | 0.28 | 1.00 | 6 | 0.46 | 1.00 |

B. Cellular component

| | | | | | | | | | | | | | | |
|---|---|---|---|---|---|---|---|---|---|---|---|---|---|---|
| Extracellular | 0005576 | 586 | 22 | 0.00 | 1.00 | 4 | 1.00 | 1.00 | 1 | 0.94 | 1.00 | 23 | 0.01 | 1.00 |
| extracellular matrix | 0005578 | 181 | 11 | 0.00 | 1.00 | 0 | 1.00 | 1.00 | 0 | 1.00 | 1.00 | 4 | 0.61 | 1.00 |
| extracellular space | 0005615 | 228 | 7 | 0.17 | 1.00 | 1 | 0.99 | 1.00 | 0 | 1.00 | 1.00 | 13 | 0.00 | 1.00 |
| Membrane | 0016020 | 1976 | 48 | 0.07 | 1.00 | 32 | 0.93 | 1.00 | 6 | 0.92 | 1.00 | 24 | 1.00 | 1.00 |
| endomembrane system | 0012505 | 112 | 2 | 0.66 | 1.00 | 4 | 0.18 | 1.00 | 0 | 1.00 | 1.00 | 1 | 0.93 | 1.00 |
| integral to membrane | 0016021 | 1549 | 26 | 0.88 | 1.00 | 21 | 0.99 | 1.00 | 5 | 0.86 | 1.00 | 18 | 1.00 | 1.00 |
| mitochondrial membrane | 0005740 | 75 | 0 | 1.00 | 1.00 | 4 | 0.06 | 1.00 | 0 | 1.00 | 1.00 | 0 | 1.00 | 1.00 |
| plasma membrane | 0005886 | 1041 | 27 | 0.09 | 1.00 | 10 | 1.00 | 1.00 | 3 | 0.87 | 1.00 | 13 | 1.00 | 1.00 |
| Cytoplasm | 0005737 | 1705 | 51 | 0.00 | 1.00 | 39 | 0.16 | 1.00 | 9 | 0.35 | 1.00 | 30 | 0.98 | 1.00 |
| Cytoplasmic vesicle | 0016023 | 59 | 2 | 0.33 | 1.00 | 0 | 1.00 | 1.00 | 0 | 1.00 | 1.00 | 2 | 0.40 | 1.00 |
| Cytoskeleton | 0005856 | 334 | 19 | 0.00 | 0.00 | 11 | 0.07 | 1.00 | 1 | 0.79 | 1.00 | 4 | 0.96 | 1.00 |
| actin cytoskeleton | 0015629 | 120 | 8 | 0.00 | 1.00 | 2 | 0.69 | 1.00 | 0 | 1.00 | 1.00 | 0 | 1.00 | 1.00 |
| microtubule cytoskeleton | 0015630 | 78 | 3 | 0.21 | 1.00 | 6 | 0.00 | 1.00 | 1 | 0.30 | 1.00 | 0 | 1.00 | 1.00 |
| Cytosol | 0005829 | 176 | 6 | 0.14 | 1.00 | 2 | 0.87 | 1.00 | 1 | 0.55 | 1.00 | 2 | 0.92 | 1.00 |
| Endoplasmic reticulum | 0005783 | 244 | 8 | 0.12 | 1.00 | 1 | 0.99 | 1.00 | 1 | 0.67 | 1.00 | 4 | 0.83 | 1.00 |
| Golgi apparatus | 0005794 | 183 | 3 | 0.72 | 1.00 | 1 | 0.98 | 1.00 | 2 | 0.20 | 1.00 | 3 | 0.80 | 1.00 |
| Mitochondrion | 0005739 | 315 | 4 | 0.89 | 1.00 | 14 | 0.00 | 1.00 | 2 | 0.42 | 1.00 | 3 | 0.98 | 1.00 |
| Ribosome | 0005840 | 61 | 0 | 1.00 | 1.00 | 1 | 0.71 | 1.00 | 1 | 0.24 | 1.00 | 1 | 0.76 | 1.00 |
| Nucleus | 0005634 | 1483 | 15 | 1.00 | 1.00 | 51 | 0.00 | 0.00 | 7 | 0.51 | 1.00 | 44 | 0.04 | 1.00 |
| nuclear membrane | 0005635 | 50 | 2 | 0.27 | 1.00 | 3 | 0.08 | 1.00 | 0 | 1.00 | 1.00 | 0 | 1.00 | 1.00 |
| nucleolus | 0005730 | 39 | 0 | 1.00 | 1.00 | 5 | 0.00 | 1.00 | 0 | 1.00 | 1.00 | 1 | 0.60 | 1.00 |
| nucleoplasm | 0005654 | 91 | 1 | 0.84 | 1.00 | 3 | 0.27 | 1.00 | 0 | 1.00 | 1.00 | 5 | 0.06 | 1.00 |
| Chromosome | 0005694 | 108 | 0 | 1.00 | 1.00 | 8 | 0.00 | 1.00 | 0 | 1.00 | 1.00 | 10 | 0.00 | 0.00 |
| Cellular—component unknown | 0008372 | 266 | 3 | 0.91 | 1.00 | 2 | 0.97 | 1.00 | 2 | 0.34 | 1.00 | 8 | 0.28 | 1.00 |

C. Molecular function

| | | | | | | | | | | | | | | |
|---|---|---|---|---|---|---|---|---|---|---|---|---|---|---|
| Apoptosis regulator activity | 0016329 | 44 | 2 | 0.22 | 1.00 | 1 | 0.59 | 1.00 | 0 | 1.00 | 1.00 | 4 | 0.02 | 1.00 |
| Binding | 0005488 | 3368 | 58 | 0.97 | 1.00 | 72 | 0.18 | 1.00 | 9 | 0.99 | 1.00 | 83 | 0.24 | 1.00 |
| lipid binding | 0008289 | 94 | 2 | 0.57 | 1.00 | 5 | 0.04 | 1.00 | 0 | 1.00 | 1.00 | 1 | 0.89 | 1.00 |
| metal ion binding | 0046872 | 631 | 22 | 0.01 | 1.00 | 6 | 0.99 | 1.00 | 0 | 1.00 | 1.00 | 12 | 0.81 | 1.00 |
| nucleic acid binding | 0003676 | 1232 | 6 | 1.00 | 1.00 | 34 | 0.02 | 1.00 | 6 | 0.49 | 1.00 | 36 | 0.08 | 1.00 |
| nucleotide binding | 0000166 | 763 | 11 | 0.91 | 1.00 | 26 | 0.00 | 1.00 | 3 | 0.68 | 1.00 | 10 | 0.99 | 1.00 |
| protein binding | 0005515 | 814 | 20 | 0.20 | 1.00 | 18 | 0.34 | 1.00 | 1 | 0.98 | 1.00 | 26 | 0.05 | 1.00 |
| receptor binding | 0005102 | 301 | 7 | 0.40 | 1.00 | 2 | 0.98 | 1.00 | 1 | 0.75 | 1.00 | 14 | 0.01 | 1.00 |
| Catalytic activity | 0003824 | 2129 | 37 | 0.89 | 1.00 | 55 | 0.01 | 1.00 | 12 | 0.21 | 1.00 | 43 | 0.90 | 1.00 |
| hydrolase activity | 0016787 | 917 | 14 | 0.90 | 1.00 | 20 | 0.35 | 1.00 | 4 | 0.61 | 1.00 | 25 | 0.22 | 1.00 |
| kinase activity | 0016301 | 409 | 5 | 0.92 | 1.00 | 16 | 0.01 | 1.00 | 3 | 0.28 | 1.00 | 2 | 1.00 | 1.00 |
| oxidoreductase activity | 0016491 | 320 | 10 | 0.11 | 1.00 | 10 | 0.10 | 1.00 | 2 | 0.42 | 1.00 | 6 | 0.76 | 1.00 |
| transferase activity | 0016740 | 706 | 8 | 0.98 | 1.00 | 22 | 0.02 | 1.00 | 5 | 0.20 | 1.00 | 8 | 1.00 | 1.00 |
| Cell adhesion molecule activity | 0005194 | 209 | 9 | 0.02 | 1.00 | 0 | 1.00 | 1.00 | 0 | 1.00 | 1.00 | 3 | 0.87 | 1.00 |
| Defense/immunity protein | 0003793 | 36 | 0 | 1.00 | 1.00 | 0 | 1.00 | 1.00 | 0 | 1.00 | 1.00 | 1 | 0.57 | 1.00 |
| activity | | | | | | | | | | | | | | |
| Enzyme regulator activity | 0030234 | 306 | 8 | 0.27 | 1.00 | 5 | 0.73 | 1.00 | 5 | 0.01 | 1.00 | 6 | 0.72 | 1.00 |
| enzyme inhibitor activity | 0004857 | 132 | 7 | 0.02 | 1.00 | 0 | 1.00 | 1.00 | 2 | 0.12 | 1.00 | 4 | 0.37 | 1.00 |
| Motor activity | 0003774 | 61 | 1 | 0.71 | 1.00 | 1 | 0.71 | 1.00 | 0 | 1.00 | 1.00 | 0 | 1.00 | 1.00 |
| Obsolete molecular function | 0008369 | 419 | 12 | 0.13 | 1.00 | 4 | 0.97 | 1.00 | 1 | 0.86 | 1.00 | 12 | 0.27 | 1.00 |
| Signal transducer activity | 0004871 | 1157 | 16 | 0.97 | 1.00 | 17 | 0.94 | 1.00 | 5 | 0.61 | 1.00 | 23 | 0.83 | 1.00 |
| receptor activity | 0004872 | 678 | 7 | 0.99 | 1.00 | 9 | 0.93 | 1.00 | 4 | 0.36 | 1.00 | 11 | 0.93 | 1.00 |
| receptor binding | 0005102 | 301 | 7 | 0.40 | 1.00 | 2 | 0.98 | 1.00 | 1 | 0.75 | 1.00 | 14 | 0.01 | 1.00 |
| receptor signaling protein | 0005057 | 127 | 2 | 0.73 | 1.00 | 4 | 0.24 | 1.00 | 0 | 1.00 | 1.00 | 0 | 1.00 | 1.00 |
| activity | | | | | | | | | | | | | | |
| Structural molecule activity | 0005198 | 333 | 17 | 0.00 | 1.00 | 5 | 0.80 | 1.00 | 3 | 0.19 | 1.00 | 4 | 0:96 | 1.00 |
| extracellular matrix structural | 0005201 | 60 | 5 | 0.01 | 1.00 | 0 | 1.00 | 1.00 | 0 | 1.00 | 1.00 | 1 | 0.76 | 1.00 |
| constituent | | | | | | | | | | | | | | |
| structural constituent of | 0005200 | 62 | 6 | 0.00 | 1.00 | 0 | 1.00 | 1.00 | 0 | 1.00 | 1.00 | 1 | 0.77 | 1.00 |
| cytoskeleton | | | | | | | | | | | | | | |
| Transcription regulator activity | 0030528 | 630 | 4 | 1.00 | 1.00 | 16 | 0.18 | 1.00 | 0 | 1.00 | 1.00 | 18 | 0.21 | 1.00 |
| transcription cofactor activity | 0003712 | 153 | 0 | 1.00 | 1.00 | 5 | 0.19 | 1.00 | 0 | 1.00 | 1.00 | 7 | 0.07 | 1.00 |
| transcription factor activity | 0003700 | 472 | 3 | 1.00 | 1.00 | 11 | 0.33 | 1.00 | 0 | 1.00 | 1.00 | 12 | 0.42 | 1.00 |
| Translation regulator activity | 0045182 | 43 | 0 | 1.00 | 1.00 | 0 | 1.00 | 1.00 | 1 | 0.18 | 1.00 | 1 | 0.64 | 1.00 |
| Transporter activity | 0005215 | 822 | 20 | 0.21 | 1.00 | 16 | 0.57 | 1.00 | 1 | 0.98 | 1.00 | 14 | 0.92 | 1.00 |
| carrier activity | 0005386 | 239 | 7 | 0.20 | 1.00 | 4 | 0.70 | 1.00 | 1 | 0.67 | 1.00 | 3 | 0.92 | 1.00 |
| electron transporter activity | 0005489 | 138 | 8 | 0.01 | 1.00 | 6 | 0.06 | 1.00 | 1 | 0.47 | 1.00 | 5 | 0.22 | 1.00 |
| ion transporter activity | 0015075 | 170 | 6 | 0.13 | 1.00 | 5 | 0.25 | 1.00 | 1 | 0.54 | 1.00 | 2 | 0.91 | 1.00 |
| protein transporter activity | 0008565 | 143 | 3 | 0.55 | 1.00 | 2 | 0.78 | 1.00 | 0 | 1.00 | 1.00 | 4 | 0.43 | 1.00 |
| Molecular_function unknown | 0005554 | 257 | 2 | 0.97 | 1.00 | 4 | 0.76 | 1.00 | 1 | 0.69 | 1.00 | 6 | 0.56 | 1.00 |

Table 14
GoMiner analysis of dysregulated genes in four immortal LFS cell lines. The genes, which were dysregulated (up- or down-regulated) during immortalization and 5aza-CdR treatment in MDAH041, MDAH087-N, MDAH087-1, MDAH087-10 cells were analyzed by GoMiner according to biological process (A), cellular component (B) and molecular function (C).
The GO categories plotted in FIG. 2 are denoted by bold font.
Total: total gene number associated with the GO term on Affymetrix HGU95av2 GeneChip ®;
Immortal: genes dysregulated during immortalization;
5aza: genes dysregulated during 5aza-CdR treatment of immortal cells.
P*: corrected p-value (p < 0.005 were rounded to 0.00; p* > 1 were reduced to 1.00)

TABLE 15
35 Genes upregulated during immortalization in gene
ontology cell proliferation category 0008283

| | Affymetrix HGU95Av2 | | Average | Fold |
|---|---|---|---|---|
| Symbol | Probe ID | LocusLink | Signal Log₂ | Change |
| BAX | 2065_s_at | 581 | 0.79 | 1.73 |
| BCAT1 | 38201_at | 586 | 2.06 | 4.18 |
| BCR | 1635_at | 613 | 0.92 | 1.89 |
| BIN1 | 32238_at | 274 | 1.04 | 2.05 |
| CDC20 | 38414_at | 991 | 1.06 | 2.09 |
| CDC25B | 1347_at | 994 | 0.64 | 1.56 |
| CDKN3 | 1599_at | 1033 | 1.05 | 2.07 |
| CENPB | 37931_at | 1059 | 0.98 | 1.97 |
| CHC1 | 37927_at | 1104 | 1.04 | 2.05 |
| CKS2 | 40690_at | 1164 | 1.02 | 2.02 |
| E2F4 | 1703_g_at | 1874 | 1.24 | 2.36 |
| EGFR | 1537_at | 1956 | 2.97 | 7.86 |
| EMP1 | 1321_s_at | 2012 | 2.58 | 5.96 |
| ERF | 38996_at | 2077 | 1.12 | 2.17 |
| IGF1R | 34718_at | 3480 | 0.86 | 1.81 |
| ILF3 | 40845_at | 3609 | 1.33 | 2.51 |
| ILF3 | 40846_g_at | 3609 | 0.91 | 1.88 |
| MAPK1 | 976_s_at | 5594 | 0.82 | 1.77 |
| MET | 35684_at | 4233 | 1.15 | 2.23 |
| MYC | 1827_s_at | 4609 | 1.14 | 2.20 |
| MYC | 1973_s_at | 4609 | 1.63 | 3.10 |
| MYC | 37724_at | 4609 | 1.31 | 2.48 |
| NOL1 | 1979_s_at | 4839 | 1.31 | 2.49 |
| NRAS | 1539_at | 4893 | 1.38 | 2.60 |
| NRP1 | 36836_at | 8829 | 1.54 | 2.91 |
| PES1 | 41869_at | 23481 | 0.87 | 1.82 |
| PLK | 37228_at | 5347 | 1.08 | 2.12 |
| PPP5C | 392_g_at | 5536 | 1.72 | 3.30 |
| PPP5C | 391_at | 5536 | 1.18 | 2.26 |
| PRIM1 | 798_at | 5557 | 1.75 | 3.35 |
| PRIM2A | 122_at | 5558 | 0.90 | 1.86 |
| RAD21 | 38114_at | 5885 | 0.77 | 1.70 |
| RAF1 | 1917_at | 5894 | 0.85 | 1.80 |
| RFC5 | 653_at | 5985 | 1.42 | 2.68 |
| SMC2L1 | 37502_at | 10592 | 1.18 | 2.27 |
| TOP1 | 1710_s_at | 7150 | 1.80 | 3.48 |
| TOP2B | 1581_s_at | 7155 | 1.42 | 2.68 |
| UBE2C | 1651_at | 11065 | 1.08 | 2.11 |
| VEGF | 1953_at | 7422 | 1.08 | 2.11 |
| VEGF | 36100_at | 7422 | 1.04 | 2.06 |
| VEGF | 36101_s_at | 7422 | 3.52 | 11.49 |

TABLE 16
Sixteen of the 19 genes identified in the wounding category
(GO: 009611) are interferon and/or cytokine regulated genes.

| | Affymetrix | | Average | | | | |
|---|---|---|---|---|---|---|---|
| | HGU95Av2 | | Signal | Fold | Table I | | |
| Symbol | Probe ID | LocusLink | Log₂ | Change | Category | IFN | Cytokine |
| CD97 | 35625_at | 976 | −0.70 | −1.63 | B | • | |
| CXCL12 | 32666_at | 6387 | −2.08 | −4.22 | B | | • |
| FGF7 | 1466_s_at | 2252 | −2.03 | −4.09 | B | | • |
| MAP2K3 | 1622_at | 5606 | −0.41 | −1.33 | B | | |
| F2R | 41700_at | 2149 | −0.79 | −1.73 | C | | |
| CCL20 | 40385_at | 6364 | 6.10 | 68.57 | D | • | • |
| CXCL2 | 37187_at | 2920 | 3.22 | 9.33 | D | | • |
| CXCL3 | 34022_at | 2921 | 3.37 | 10.36 | D | | • |
| CXCL6 | 35410_at | 6372 | 2.36 | 5.12 | D | | • |
| GAGE1 | 31497_at | 2543 | 1.92 | 3.79 | D | | |
| IL1B | 39402_at | 3553 | 2.95 | 7.75 | D | | • |
| IL8 | 35372_r_at | 3576 | 3.20 | 9.20 | D | • | • |
| MAP2K3 | 2075_s_at | 5606 | 0.72 | 1.64 | D | | |
| MICB | 35937_at | 4277 | 0.91 | 1.88 | D | • | |
| MYD88 | 38369_at | 4615 | 0.96 | 1.95 | D | • | |
| NFKB1 | 1377_at | 4790 | 0.92 | 1.89 | D | | |
| NFKB1 | 38438_at | 4790 | 0.87 | 1.83 | D | | |
| NFKB1 | 1378_g_at | 4790 | 0.61 | 1.53 | D | | |
| NMI | 36472_at | 9111 | 0.92 | 1.90 | D | • | |
| SAA1 | 33272_at | 6288 | 2.74 | 6.66 | D | | |
| TAP1 | 40153_at | 6890 | 1.48 | 2.79 | D | • | |

TABLE 17
Genes with decreased expression during immortalization that are in GO categories structural
molecular activity genes (GO: 0005198), cell adhesion molecular activity (GO: 0005194)
and cytoskeletal category (GO: 0005856). Sixteen of the 24 genes from structural
molecular activity genes (GO: 0005198), and 1 of the 9 genes in the cell adhesion molecular
activity (GO: 0005194), overlap with the genes in the cytoskeletal category (GO: 0005856)

| | Affymetrix | | Average | | | | |
|---|---|---|---|---|---|---|---|
| | HGU95Av2 | | Signal | Fold | Adhesion | Structural | Cyto [symbol] |
| Symbol | Probe ID | LocusLink | Log₂ | Change | (GO: 0005194) | (GO: 0005198) | (GO [symbol] |
| ACTA2 | 32755_at | 59 | −2.48 | −5.57 | | • | |
| ACTC | 39063_at | 70 | −6.09 | −67.90 | | • | |
| ACTR1A | 40052_at | 10121 | −0.58 | −1.50 | | • | |
| ADD1 | 32145_at | 118 | −0.48 | −1.40 | | • | |
| ARPC1B | 39043_at | 10095 | −1.10 | −2.14 | | • | |
| BPAG1 | 32780_at | 667 | −0.54 | −1.45 | • | • | |
| CAP2 | 33405_at | 10486 | −1.34 | −2.53 | | • | |
| CAPG | 38391_at | 822 | −2.50 | −5.65 | | | |
| CD97 | 35625_at | 976 | −0.70 | −1.63 | • | | |
| CD99 | 41138_at | 4267 | −1.35 | −2.55 | • | | |
| CNN1 | 34203_at | 1264 | −3.39 | −10.50 | | | |
| COL4A1 | 39333_at | 1282 | −2.82 | −7.04 | | • | |
| COL4A2 | 36659_at | 1284 | −1.93 | −3.82 | | • | |
| CRYAB | 32242_at | 1410 | −3.95 | −15.41 | | • | |
| CRYAB | 32243_g_at | 1410 | −3.84 | −14.32 | | • | |
| DSP | 36133_at | 1832 | −3.91 | −15.04 | | • | |
| ECM1 | 37600_at | 1893 | −1.77 | −3.40 | | • | |
| ELN | 39098_at | 2006 | −3.36 | −10.23 | | • | |
| EMS1 | 39861_at | 2017 | −1.35 | −2.56 | | | |
| ENG | 32562_at | 2022 | −0.93 | −1.90 | • | | |
| EPB41L3 | 41385_at | 23136 | −4.68 | −25.66 | | • | |
| FARP1 | 32148_at | 10160 | −2.27 | −4.82 | | | |
| FBLN5 | 39038_at | 10516 | −1.70 | −3.24 | • | | |
| FEZ1 | 37743_at | 9638 | −3.53 | −11.58 | | | |
| FEZ2 | 38651_at | 9637 | −0.84 | −1.79 | | | |
| GSN | 32612_at | 2934 | −1.27 | −2.42 | | • | |
| ITGA1 | 37484_at | 3672 | −1.90 | −3.72 | • | | |
| ITGA7 | 36892_at | 3679 | −2.00 | −3.99 | • | | |
| KNS2 | 39057_at | 3831 | −0.69 | −1.62 | | | |
| MAP1A | 35917_at | 4130 | −1.30 | −2.46 | | • | |
| ME1 | 31824_at | 4199 | −1.38 | −2.61 | • | | |
| MYL9 | 39145_at | 10398 | −1.49 | −2.81 | | • | |
| NID | 35366_at | 4811 | −1.25 | −2.38 | • | • | |
| NOTCH3 | 38750_at | 4854 | −3.66 | −12.62 | | • | |
| PEA15 | 32260_at | 8682 | −1.34 | −2.53 | | | |
| SGCD | 41378_at | 6444 | −2.59 | −6.00 | | | |
| SGCD | 34993_at | 6444 | −2.07 | −4.21 | | | |
| SGCD | 34991_at | 6444 | −2.00 | −4.00 | | | |
| SPTAN1 | 33833_at | 6709 | −0.72 | −1.64 | | • | |
| STOM | 40419_at | 2040 | −1.88 | −3.69 | | | |
| STX6 | 41663_at | 10228 | −0.77 | −1.71 | | | |
| STX7 | 38774_at | 8417 | −0.84 | −1.79 | | | |
| TEK | 1596_g_at | 7010 | −4.15 | −17.71 | | • | |
| TPM2 | 32313_at | 7169 | −1.14 | −2.21 | | • | |
| TPM2 | 32314_g_at | 7169 | −0.83 | −1.78 | | • | |
| TUBB | 39331_at | 7280 | −1.05 | −2.07 | | • | |
| VAMP3 | 35783_at | 9341 | −0.47 | −1.39 | | | |
| VAMP5 | 32533_s_at | 10791 | −1.71 | −3.27 | | | |
| VIL2 | 40103_at | 7430 | −1.19 | −2.29 | | • | |

### REFERENCES

Agrawal S, Agarwal M L, Chatterjee-Kishore M, Stark G R and Chisolm G M. (2002) Mol. Cell. Biol., 22, 1981-1992.
Baylin S B, Esteller M. Rountree M R. Bachman K E. Schuebel K and Herman J G. (2001). Hum. Mol. Genet., 10, 687-692.
Bechter O E, Eisterer W, Diaska M, Kuhr T and Thaler J. (2002). Exp. Hematol., 30, 26-33.
Bender C M, Pao M M and Jones P A. (1998). Cancer Res., 58, 95-101.
Berube N G, Smith J R and Pereira-Smith O M. (1998). Am. J. Hum. Genet., 62,1015-1019.
Bischoff F Z, Yim S O, Pathak S, Grant G, Siciliano M J, Giovanella B C, Strong L C and Tainsky M A. (1990). Cancer Res., 50, 7979-7984.
Brown R and Strathdee G. (2002). Expert Opin. Invest Drugs 11, 747-754.
Bryan T M, Englezou A, Dalla-Pozza L, Dunham M A and Reddel R R.(1997). Nat. Med., 3, 1271-1274.
Bryan T M, Englezou A, Gupta J, Bacchetti and Reddel R R. (1995). EMBO J., 14, 4240-4248.
Carpten J, Nupponen N, Isaacs S, Sood R, Robbins C, Xu J, Faruque M, Moses T, Ewing C, Gillanders E, Hu P, Bujnovszky P, Makalowska I, Baffoe-Bonnie A, Faith D, Smith J, Stephan D, Wiley K, Brownstein M, Gildea D, Kelly B, Jenkins R, Hostetter G, Matikainen M, Schleutker J, Klinger K, Connors T, Xiang Y, Wang Z, De Marzo A, Papadopoulos N, Kallioniemi O P, Burk R, Meyers D, Gronberg H, Meltzer P, Silverman R, Bailey-Wilson J, Walsh P, Isaacs W and Trent J. (2002). Nat. Genet., 30,181-184.
Choubey D and Lengyel P. (1995). J. Biol. Chem., 270, 6134-6140.
Dessain S K, Yu H, Reddel R R, Beijersbergen R L and Weinberg R A. (2000). Cancer Res., 60, 537-541.
DeYoung K L, Ray M E, Su Y A, Anzick S L, Johnstone R W, Trapani J A, Meltzer P S and Trent J M. (1997). Oncogene 15, 453-457.
Dimri G P, Lee X, Basile G, Acosta M, Scott G, Roskelley C, Medrano E E, Linskens M, Rubelj I and Pereira-Smith O et al. (1995). Proc. Natl. Acad. Sci. USA 92, 9363-9367.
Draghici S. (2002). Drug Discov. Today 7, S55 .S63.
D'Souza S, Xin H, Walter and Choubey D. (2001). J. Biol. Chem., 276, 298-305.
Duncan E L, Wadhwa R and Kaul S C. (2000). Biogerontology 1,103-121.
Dunham M A, Neumann A A, Fasching C L and Reddel R R. (2000). Nat. Genet., 26,447-450.
Fairweather N F, Lyness V A and Maskell D J. (1987). Infect Immun., 55, 2541-2545.
Gardiner-Garden M and Frommer M.(1987). J. Mol. Biol., 196, 261-282.
Gollahon L S, Kraus E, Wu T A, Yim S O, Strong L C, Shay J W and Tainsky M A. (1998). Oncogene 17, 709-717.
Graham F L, Smiley J, Russell W C and Nairn R. (1977). J. Gen. Virol., 36, 59-74.
Gutman A and Wasylyk B. (1991). Trends Genet., 7, 49-54.
Harada H, Kitagawa M, Tanaka N, Yamamoto H, Harada K, Ishihara M and Taniguchi T. (1993). Science 259, 971-974.
Harada M, Li Y F, E I-Gamil M, Ohnmacht G A, Rosenberg S A and Robbins P F. (2001). J. Immunother., 24, 323-333.
Hay Flick L. (1976). N. Engl. J. Med., 295,1302-1308.
Herman J G and Baylin S B. (2000). Curr. Top. Microbiol. Immunol., 249, 35-54.
Huschtscha L I and Holliday R. (1983). J. Cell. Sci., 63, 77-99.
Jagus R, Joshi B and Barber G N. (1999). Int. J. Biochem. Cell. Biol., 31,123-138.
Jay P, Rougeulle C, Massacrier A, Moncla A, Mattei M G, Malzac P, Roeckel N, Taviaux S, Lefranc J L, Cau P, Berta P, Lalande Mand Muscatelli F. (1997). Nat. Genet.,17, 357-361.
Karpf A R, Peterson P W, Rawlins J T, Dalley B K, Yang Q, Albertsen H and Jones D A. (1999). Proc. Natl. Acad. Sci. USA 96,14007-14012.
Kerr M K, Martin M and Churchill G A. (2000). J. Comput Biol., 7, 819-837.
Liang G, Gonzales F A, Jones P A, Orntoft T F and Thykjaer T. (2002). Cancer Res., 62, 961-966.
Malkin D. (1994). Biochim. Biophys. Acta 1198, 197-213.
Malkin D, Li F P, Strong L C, Fraumeni Jr J F, Nelson C E, Kim D H, Kassel J, Gryka M A, Bischoff F Z and Tainsky M A et al. (1990). Science 250, 1233-1238.
Mori Y, Yin J, Rashid A, Leggett B A, Young J, Simms L, Kuehl P M, Langenberg P, Meltzer S J and Stine O C. (2001). Cancer Res., 61, 6046-6049.
Nakada Y, Taniura H, Uetsuki T, Inazawa J and Yoshikawa K. (1998). Gene, 213, 65-72.
Newell-Price J, Clark A J and King P. (2000). Trends Endocrinol. Metab. 11, 142-148.
Noda A, Ning Y, Venable S F, Pereira-Smith O M and Smith J R.(1994). Exp. Cell. Res. 211, 90-98.
Pereira-Smith O M and Smith J R. (1983). Science, 221, 964-966.
Pereira-Smith O M and Smith J R. (1988). Proc. Natl. Acad. Sci. USA 85, 6042-6046.
Rogan E M, Bryan T M, Hukku B, Maclean K, Chang A C, Moy E L, Englezou A, Warneford S G, Dalla-Pozza L and Reddel R R. (1995). Mol. Cell. Biol., 15, 4745-4753.
Schwarze S R, DePrimo S E, Grabert L M, Fu V X, Brooks J D and Jarrard D F. (2002). J. Biol. Chem., 277, 14877-14883.
Shay J W, Tomlinson G, Piatyszek M A and Gollahon L S. (1995). Mol. Cell. Biol.,15, 425-432.
Shelton D N, Chang E, Whittier P S, Choi D and Funk W D. (1999). Curr. Biol., 9, 939-945.
Vogt M, Haggblom C, Yeargin J, Christiansen-Weber T and Haas M. (1998). Cell Growth Differ., 9,139-146.
Wen Y, Yan D H, Wang B, Spohn B, Ding Y, Shao R, Zou Y, Xie K and Hung M C. (2001). Cancer Res. 61, 7142-7147.
Willman C L, Sever C E, Pallavicini M G, Harada H, Tanaka N, Slovak M L, Yamamoto H, Harada K, Meeker T C and List A F et al. (1993). Science 259,968-971.
Xin H, D'Souza S, Fang L, Lengyel P and Choubey D. (2001). Oncogene 20, 6828-6839.
Young J I and Smith J R. (2001). J. Biol. Chem., 276, 19610-19616.
Hayflick L. and Moorhead, P S. 1961. The limited in vitro lifetime of human diploid cell strains. Exp Cell Res 25:585-621
Vogt M, Haggblom C, Yeargin J, Christiansen-Weber T, Haas M. Independent induction of senescence by p16INK4a and p21CIP1 in spontaneously immortalized human fibroblasts. Cell Growth Differ. 19989:139-46.
Counter, C. M., Avilion, A. A., LeFeuvre, C. E., Steward, N. G., Greider, C. W., Harley, C. B., and Bacchetti, S. 1992. Telomere shortening associated with chromosome instability is arrested in immortal cells which express telomerase activity.
Bryan, T. M., Englezou, A., Gupta, J., Bacchetti, S. and Reddel, R. R. (1995) Telomere elongationin immortal human cells without detectable telornerase activity. EMBO J., 14, 4240-4248.
Kaul, S. C., Duncan, E. L., Englezou, A., Takano, S., Reddel, R. R., Mitsui, Y., Wadhwa, R. 1998. Malignant transformation of NIH3T3 cells by overexpression of mot-2 protein. Oncogene. 17:907-11.
Wadhwa, R., Kaul, S. C., Mitsui, Y. 1994. Cellular mortality to immortalization: mortalin. Cell Struct Funct. 19:1-10.
Pereira-Smith, O. M., Smith, J. R. 1988. Genetic analysis of indefinite division in human cells: Identification of four complementation groups. Proc. Natl. Acad. Sci. 85:6042-46.
Leung, J. K., Pereira-Smith, O. M. 2001. Identification of genes involved in cell senescence and immortalization: potential implications for tissue aging. Novartis Found Symp. 235:105-10.
Holliday, R. 1990. Mechanisms for the control of gene activity during development. Bioi. Rev. 65:431-71.
Bird, A. 1992. The essentials of DNA methylation. Cell. 70,5-8.
Boyes, J., Bird,A., 1991. DNA methylation inhibits transcription indirectly via a methyl-CpG binding protein. Cell. 64:1123-1124.
Jaenisch R. 1997. DNA methylation and cancer. Hum. Mol. Genet. 3:1487-95.
Barlow, D., Stoger, R., Herrmann, B. G., Stito, K., Schweifer, N. 1991. The mouse insulin-like growth factor type-2 receptor is imprinted and closely linked to the Tme locus. Nature. 349:84-87.
Counts, J. L., Sarmiento, J. I., Harbison, M. L., Downing, J. C., McClain, R. M., Goodman, J. t. 1996. Cell proliferation and global methylation status changes in mouse liver after Phenobarbital and/or choline-devoid, methionine-deficient diet administration. Carcinogenesis 17: 1251-57.
Ming, J., Blagowidow, N., Knoll, J., rollings, L., Fortina, P., et al. 2000. Submicroscopic deletion in cousins with Prader-Wili syndrome causes a gradmatrilineal inheritance pattern: effects of imprinting. Am. J. Med. Genet. 92:19-24.
Makos, M., Nelkin, B. D., Chazin, V. R., Cavenee, W. K., Brodeur, G. M., Baylin, S. B. 1993. DNA hypermethylation is associated with 17p allelic loss in neural tumors. Cancer Res. 53:2715-18.
Makos, M., Nelkin, B. D., Reiter, R. E., Gnarra, J. R., Brooks, J. et al. 1993. Regional hypermethylation at D17S5 precedes 17p structural changes in the progression 0 frenal tumors. Cancer Res. 53:2719-22.
Magewu, A. N. and Jones, P. A. 1994. Ubiquitous and tenacious methylation of the CpG site in codon 248 of the p53 gene may explain its frequent appearance as a mutational hot spot in human cancer. Mol. Cell. Bioi., 14, 4225-4232.
Greenblatt, M. S., Bennett, W. P., Hollstein, M. , Harris, C. C. 1994. Mutations in the p53 tumor suppressor gene: Clues to cancer etiology and molecular pathogenesis. Cancer Res., 54, 4855-4878.
Baylin, S. B., Esteller, M., Rountree, M. R., Bachman, K. E., Schuebel, K., Herman, J. G. 2001. Aberrant patterns of DNA methylation, chromatin formation and gene expression in cancer. Human Molecular Genetics. 10(7): 687-692.
Wales, M. M., Biel, M. A., el Deiry, W., Nelkin. B. D., Issa, J. P., Cavenee, W. K., et al. 1995. p53 activates expression of HIC-1, a new candidate tumor suppressor gene on 17p13.3. Nat Med. 1 :570-7.
Costello, J. F., Fruhwald, M. C., Smiraglia, D. J., Rush, L. J., Robertson, G. P., Gao, X., et al. 2000. Aberrant CpG-island methylation has non-random and tumour-type-specific patterns. Nat Genet. 24: 132-8.
Toyota, M., Ahuja, N., Ohe-Toyota, M., Herman, J. G., Baylin, S. B., Issa, J. P. 1999. CpG island methylator phenotype in colorectal cancer. Proc Natl Acad Sci. 96:8681-6.
Liang, G., Gonzales, F. A., Jones, P. A., Orntoft, T. F., Thykjaer, T. 2002. Analysis of gene induction in human fibroblast and bladder cancer cells exposed to the methylation inhibitor 5-aza-2′-deoxycytidine. Cancer Res 62(4):961-6.
Peris, K., Stanta, G., Fargnoli, M. C″ Bonin, S., Felli, A., Amantea, A., Chimenti, S. 1999. Reduced expression of CDKN2a/p16INK4a in mycosis fungoides. Arch Dermatol Res. 291 :207-211.
Belinsky, S. A., Nikula, K. J., Palmisano, W. A., Michels, R., Saccomanno, G., et al. 1998. Aberrant methylation of p161NK4a is an early event in lung cancer and a potential biomarker for early diagnosis. Proc. Natl. Acad. Sci. 95:11,891-96.
Foster, S, A., wong, D. J., Barrett, M. T. and Gallowway, D. A. 1998. Inactivation of p16 in human mammary epithelial cells by CpG island methylation. Mol. Cell. Biol. 18:1793-1801.
Loughran, 0., Malliri, A., Owens, D, et al. 1996. Association of CDKN2Np161NK4A with human head and neck keratinocyte replicative senescence: relationship of dysfunction to immortality and neoplasia. Oncogene. 13: 561-568.
Brenner, A. J., Stampfer, M. R. and Aldaz, C. M. 1998. Increased p16 expression with first senescence arrest in human mammary epithelial cells and extended growth capacity with p16 inactivation. Oncogene. 17: 199-205.
Kiyono, T., Foster, S. A.,Koop, J. I., McDougall, J. K., Galloway, D. A., Klingelhutz, A. J. 1998. Both rb/p16INK4a inactivation and telomerase activity are required to immortalization of human epithelial cells. Nature. 396(6706):84-8.
Counts, J. L., Goodman, J.I. 1995. Alterations in DNA methylation may playa variety of roles in carcinogenesis. Cell. 83: 13-15.
Baylin, S. B., Herman, J. G. 2001. Promoter hypermethylation-can this change alone ever designate true tumor suppressor gene function? J Nat Can Inst. 93:664-665.
Malkin, 0., Li, F. P., Strong, L. C., Fraumeni, J. F. Jr, Nelson, C. E., Kim, D. H., Kassel, J., Gryka, M. A., Bischoff, F. Z., Tainsky, M. A., Friend, S. H., 1990. Germ line p53 mutations in a familial syndrome of breast cancer, sarcomas, and other neoplasms. Science 250: 1233-8.
Gollahon, L. S., Kraus, E., Wu, T. A., Yim, S. O., Strong, L. C., Shay, J. W., Tainsky, M. A. 1998. Telomerase activity during spontaneous immortalization of Li-Fraumeni syndrome skin fibroblasts. Oncogene. 17:709-17.
Bischoff, F., Yim, S. 0., Pathak, S., Grant, G., Siciliano, M. J., Giovanella, B. C., Strong, L. C., and Tainsky, M. A. Spontaneous immortalization of normal fibroblasts from patients with Li-Fraumeni cancer syndrome: Aneuploidy and Immortalization. Cancer Res 50:7979-7984, 1990.
Bischoff, F., Strong, L. C., Yi-, S. O., Pathak, S., Pratt, D. R., Grant, G., Siciliano, M. J., Giovanella, B. C., and Tainsky, M. A. Tumorigenic transformation of spontaneously immortalized fibroblasts from patients with a familial cancer syndrome. Oncogene 6:183-186,1991.
Platanias, L. C. 1995. Interferons. Laboratory to clinic investigations. Curr Opin Oncol. 7:560.
Platanias, L. C., Fish, E. N. 1999. Signaling pathways activated by interferons. Experimental hematology. 27: 1583-1592.
Imada, K., Leonard, W. J. 2000. The Jak-STAT pathway. Molecular Immunology. 37: 1-11.
Leonard, W. J., O'Shea, J. J., 1998. Jaks and STATs: Biological implications. Annu. Rev. Immunol. 16, 293-322.
Uddin, S., Gardziola, C., Dangat, A., Yi, T., Platanias, L. C. 1996. Interaction of the c-cbl proto-oncogene product with the Tyk-2 protein tyrosine kinase. Biochem Biophys Res Commun 225:883.
Takaoka, A., Tanaka, N., Mitani, Y., Miyazaki, T., Fujii, H., Sato, M., Kovarik, P., Decker, T., Schlessinger, J., Tanigushi, T. 1999. Protein tyrosine kinase Pyk2 mediates the Jak-dependent activation of MAPK and STAT1α in IFNy, but not IFNα, signaling. EMBO J. 18:2480.
Pitha, P. M. 2000 Introduction: interferons' connection to cancer. Seminars in Cancer Biology. 10: 69-72.
Karpf, A. R., Peterson, P. W., Rawlins, J. T., Dalley, 8.K., Yang, Q., Albertsen, H., Jones, D. A. 1999. Inhibition of DNA methyltransferase stimulates the expression of signal transducer and activator of transcription 1, 2, and 3 genes in colon tumor cells. Proc Natl Acad Sci. 96(24): 14007-12.
Agrawal S, Agarwal M L, Chatterjee-Kishore M, Stark G R, Chisolm G M, 2002. STAT1α-dependent, p53-independent expression of p21 (waf1) modulates oxysterol-induced apotosis. Mol Cell Biol 22(7): 1981-92.
Chin Y E, Kitagawa M, Kuida K, Flavell R A, Fu X Y. 1997. Activation of the STAT signaling pathway can cause expression of caspase 1 and apoptosis. Mol Cell Biol. 17(9):5328-37.
Xu X, Fu X Y, Plate J, Chong A S. 1998. IFN-gamma induces cell growth inhibition by Fas-mediated apoptosis: requirement of STAT1A protein for up-regulation of Fas and FasL expression. Cancer Res. 58:2832-7.
Kaplan, D. H., Shankaran, V., Dighe, A. S., Stockert, E., Aguet, M., Old, L. J., Schreiber, R. D. 1998. Demonstration of an interferon gamma-dependent tumor surveillance system in immunocompetent mice. Proc. Natl. Acad. Sci. 95:7556-61.
Huang, S., Bucana, C. D., Arsdall, M. V., Fidler, I. J. 2000. STAT1α negatively regulates angiogenesis, tumorigenicity and metastasis of tumor cells. Oncogene. 21 (16):2504-12.
Altman, R. S., Raychaudhuri, S. 2001. Whole-genome expression analysis: challenges beyond clustering. Current opion in structural biology. 11 :340-7.
Esteller, M., Corn, P. G., Baylin, S. B., Herman, J. G. 2001. A gene hypermethylation profile of human cancer. Cancer Res. 61 (8):3225-9.
Hsieh, C. L. 1994. Dependence of transcriptional repression on Cpg methylation density. Mol Cell Biol. 14: 5487-94.
Vertino, P. M., yen, R. W., Gao, J. and Baylin, S. B. 1996. De novo methylation of CpG island sequences in human fibroblasts overexpressing DNA (cytosine-5-)-methyltransferase. Mol Cell Biol. 16:4555-65.
Graff, J. R., Herman, J. G., Myohanen, S., Baylin, S. B. and Vertino, P. M. 1997. Mapping patterns of CpG island methylation in normal and neoplastic cells implicates both upstream and downstream regions in de novo methylation. J Biol. Chem. 272:22322-29.
Galm, O., Rountree, M. R., Bachman, K. E., Jair, K. W., Baylin. S. B., Herman, J. G. 2002. Enzymatic regional methylation assay: a novel method to quantify regional CpG methylation density. Genome Res. 12(1): 153-7.
Herman, J. G., Graff, J. R., Myohanen, S., Nelkin, B. D., 8aylin,. S. B. 1996. Methylation-specific PCR: A novel PCR assay for methylation status of CpG islands. Proc Natl Acad Sci. 93:9821-6.
Herrero C, Sebastian C, Marques L, Comalada M, Xaus J, Valledor A F, IIoberas J, Celada A. Immunosenescence of macrophages: reduced MHC class 11 gene expression. Exp Gerontol. 2002 37:389-94 Lio 0, Scola L, Crivello A, Bonafe M, Franceschi C, Olivieri F, Colonna-Romano G, Candore G, Caruso C. Allele frequencies of +874T→A single nucleotide polymorphism at the first intron of interferon-gamma gene in a group of Italian centenarians. Exp Gerontol. 2002 37:315-9.

1. A diagnostic tool for use in diagnosing diseases, said tool comprising detection means for detecting markers which determine gene expression changes that are related to cellular immortalization, the presence of said markers being indicative of a disease.

2. The diagnostic tool according to claim 1, wherein the disease is selected from the group consisting essentially of cancer, infectious diseases, and aging.

3. The diagnostic tool according to claim 1, wherein said detection means is selected from the group consisting essentially of an assay, a slide, and a filter combination.

4. The diagnostic tool according to claim 1, wherein said marker is selected from the group consisting essentially of genes of the IFN pathway, and methylation changes involved in cellular immortalization.

5. A method of identifying markers of disease and aging by analyzing a microarray for molecular targets of cancer, which determine gene expression changes that are related to cellular immortalization.

6. The method according to claim 5, wherein said analyzing step includes normalizing the results of the analysis.

7. Molecular markers of disease identified by the method of claim 5.

8. The molecular markers according to claim 7, wherein said markers are selected from the group consisting essentially of genes of the IFN pathway, and gene expression changes involved in cellular immortalization.

9. A treatment of disease, said treatment comprising a compound that modulates a marker of disease identified by the method of claim 5.

10. Therapeutics for modulating molecular markers identified by the method of claim 5.

11. The therapeutics according to claim 10, wherein said therapeutics downregulate the markers.

12. The therapeutics according to claim 10, wherein said therapeutics upregulate the markers.

13. A tool for interpreting results of a microarray, said tool comprising a computer program for analyzing the results of the microrarray.

14. A method of creating an array of markers for diagnosing the presence of disease, said method comprising the steps of:
microarraying sera obtained from a patient to obtain molecular markers of disease; and
detecting markers which determine gene expression changes that are related to cellular immortalization, the markers are present only in the sera of patients with a specific disease thereby detecting molecular markers for use in diagnosing disease.

15. The method according to claim 14, wherein said detecting step includes normalizing data obtained during microarraying.

16. The method according to claim 15, wherein said normalizing step includes analyzing the intersection of subsets of genes that are differentially regulated by the microarraying.

17. The method according to claim 16, wherein the normalizing step includes confirming that the genes identified in the intersection are involved in immortalization.

18. The method according to claim 17, wherein said confirming step includes performing microarray hybridization and Q-RT-PCR.

19. The method according to claim 18, wherein said confirming step includes determining whether the genes detected are involved in immortalization.

20. The method according to claim 19, wherein said determining step includes creating a hierarchal map.
